(19) United States
(12) Patent Application Publication
(10) Pub. No.: US 2019/0111168 A1
(43) Pub. Date: Apr. 18, 2019

(54) PHOTOSENSITIZER DISPERSION, AND USE THEREOF

(71) Applicants: Universität Regensburg, Regensburg (DE); Universitätsklinikum Regensburg, Regensburg (DE)

(72) Inventors: Wolfgang BÄUMLER, Langquaid (DE); Christiane JUNG, Dorfkemmathen (DE); Burkhard KÖNIG, Lappersdorf (DE); Werner KUNZ, Regensburg (DE); Eva MÜLLER, Bad Kötzting (DE); Andreas SPÄTH, Regensburg (DE)

(73) Assignees: Universität Regensburg, Regensburg (DE); Universitätsklinikum Regensburg, Regensburg (DE)

(21) Appl. No.: 16/089,626

(22) PCT Filed: Mar. 31, 2017
(86) PCT No.: PCT/EP2017/057763

§ 371 Date: Sep. 28, 2018

(30) Foreign Application Priority Data
Apr. 1, 2016 (EP) 16163551.1

(51) Int. Cl. A61L 2/08; A61L 2/10; A61K 41/00; B65B 55/08; A23L 3/3454; A23L 3/26; C02F 1/50; C02F 1/30

(52) U.S. Cl. CPC A61L 2/088; A61L 2/10; A61L 2/084; A61K 41/0071; B65B 55/08; A23L 3/3454; A23V 2002/00; C02F 1/50; C02F 1/30; A61L 2202/21; A61L 2202/23; A61L 2202/26; A61L 2202/25; A23L 3/26

(57) ABSTRACT

A photosensitizer-containing dispersion, and use thereof.

The present invention relates to a photosensitizer-containing dispersion and to its use.

The occurrence of more and more multi-resistant bacterial isolates has meant that treating bacterial diseases has become more difficult. Increasingly strict hygiene standards and a global proliferation of nosocomial infections have sparked an interest in novel preparations, methods and applications which could inhibit the proliferation of multi-resistant germs.

The search for alternatives to antibiotic therapy is of vital importance to the treatment of infections which are caused by bacteria, for example, in particular as a result of the identification and increasing occurrences of vancomycin-resistant bacterial strains (VRSA), as early as 2002 in Japan and in the USA. In Europe, the first VRSA isolate from a patient was recorded in Portugal in 2013.

The increase in resistance to fungal infections as regards antifungal preparations further heightens the problem in the treatment of superficial infections. The clinical consequence of resistance to antifungal preparations is exhibited by failure of the treatment, most particularly in immunosuppressed patients.

New approaches to controlling resistant or multi-resistant disease-causing pathogens are thus on the one hand the search for novel antidotes, for example antibiotics or antimycotics, and on the other hand the search for alternative possibilities for inactivation.

The photodynamic inactivation of microorganisms has proved to be an alternative method. Two photooxidative processes play a decisive role in the photodynamic inactivation of microorganisms.

A photosensitizer is excited by light of a specific wavelength. The excited photosensitizer can cause the formation of reactive oxygen species (ROS), whereupon on the one hand radicals, for example superoxide anions, hydrogen peroxide or hydroxyl radicals, and/or on the other hand excited molecular oxygen, for example singlet oxygen, may be formed.

In both reactions, the photooxidation of specific biomolecules which are in the direct vicinity of the reactive oxygen species (ROS) is predominant. In this regard, in particular, lipids and proteins are oxidized which, for example, are components of the cell membrane of microorganisms. The destruction of the cell membrane again brings about the inactivation of the relevant microorganisms. A similar elimination process occurs in viruses and fungi.

As an example, singlet oxygen preferentially attacks molecules which are sensitive to oxidation. Examples of oxidation-sensitive molecules are molecules which contain double bonds or oxidation-sensitive groups such as phenols, sulphides or thiols. Unsaturated fatty acids in the membranes of bacteria are particularly prone to damage.

However, unfortunately, many known photosensitizers from the prior art exhibit an unsatisfactory wettability of hydrophobic surfaces.

Thus, a photosensitizer-containing composition should be provided which is guaranteed to be simple to apply and in particular, at the same time, which exhibits good wettability even on hydrophobic surfaces.

Furthermore, the photosensitizer-containing composition should preferably have improved adhesion of the photosensitizer following application to a surface.

Furthermore, preferably, the effectiveness of the photosensitizer should be improved.

In this regard, the photosensitizer-containing composition should essentially not inhibit excitement of the photosensitizer molecules contained in the composition by light of a specific wavelength.

The objective of the present invention is achieved by means of the provision of a photosensitizer-containing dispersion as claimed in claim 1, wherein the dispersion comprises:
(a) at least one photosensitizer,
(b) at least one liquid polar phase, and
(c) at least one surfactant

wherein the dispersion comprises and is preferably constituted by a microemulsion, a gel or a mixture thereof, at a temperature in the range 2° C. to 50° C. and a pressure in the range 800 to 1200 mbar.

Preferred embodiments of the dispersion in accordance with the invention are defined in claims 1 to 14.

The objective of the present invention is furthermore achieved by means of the use of a dispersion as claimed in one of claims 1 to 14 for the inactivation of microorganisms, which are preferably selected from the group consisting of viruses, archaeae, bacteria, bacterial spores, fungi, fungal spores, protozoa, algae and blood-borne parasites. This use may be medical or non-medical.

Preferred embodiments of the use in accordance with the invention are specified in the dependent claims 16 to 18.

The objective of the present invention is furthermore achieved by means of the provision of a method for the inactivation of microorganisms, which are preferably selected from the group consisting of viruses, archaeae, bacteria, bacterial spores, fungi, fungal spores, protozoa, algae and blood-borne parasites, wherein the method comprises the following steps:

(A) bringing the microorganisms into contact with a photosensitizer-containing dispersion as claimed in one of claims 1 to 14, and

(B) irradiating the microorganisms and the at least one photosensitizer with electromagnetic radiation of a suitable wavelength and energy density.

Preferably, the method in accordance with the invention is carried out in order to inactivate microorganisms by the photodynamic therapy of a patient or by the photodynamic decontamination of a surface of an article or an area, or by the photodynamic decontamination of a liquid, preferably by the photodynamic decontamination of a surface of an article or by the photodynamic decontamination of a liquid.

A photosensitizer-containing dispersion in accordance with the invention comprises:

(a) at least one photosensitizer,

(b) at least one liquid polar phase, and

(c) at least one surfactant, and

wherein the dispersion comprises and is preferably constituted by a microemulsion or a gel or a mixture thereof, preferably a microemulsion or a gel, at a temperature in the range 2° C. to 50° C. and a pressure in the range 800 to 1200 mbar.

The inventors have established that, by providing a photosensitizer-containing dispersion in accordance with the invention, a plurality of different categories of photosensitizer can be effectively applied, even to hydrophobic surfaces. Advantageously, in this manner, the quantity of photosensitizer which is necessary for photodynamic inactivation can be applied to the surfactant to be treated.

Furthermore, the dispersion in accordance with the invention has enough wettability for a variety of categories of photosensitizer even on hydrophobic surfaces, so that the photosensitizer-containing dispersion in accordance with the invention, and thus the at least one photosensitizer contained therein can, as is preferable, be distributed evenly over the surface to be decontaminated and preferably remains in place following application.

This advantageously ensures that, following irradiation of the surface to be decontaminated with electromagnetic radiation of a suitable wavelength and energy density, preferably in the presence of oxygen and/or an oxygen-donating compound, microorganisms adhering to the surface to be decontaminated are reliably inactivated.

Furthermore, the inventors have surprisingly discovered that the dispersion in accordance with the invention does not reduce the photodynamic activity of the at least one photosensitizer contained therein.

The photosensitizer-containing dispersion in accordance with the invention exhibits low to no turbidity, whereupon incident electromagnetic radiation is hardly attenuated or is not attenuated at all.

In this manner, the use of the at least one photosensitizer in the dispersion in accordance with the invention, compared with the use of the pure photosensitizer, surprisingly leads to no significant reduction in the yield of reactive oxygen species and/or singlet oxygen.

As high a yield of reactive oxygen species or singlet oxygen as possible is desirable for antimicrobial effectiveness in photodynamic therapy or in the photodynamic cleaning of surfaces or liquids.

Greater turbidity results in a significant reduction in the energy of the incident electromagnetic radiation. Furthermore, the occurrence of quenching phenomena within a photosensitizer composition following irradiation with electromagnetic radiation of a suitable wavelength and energy density results in a release of the energy absorbed, normally by the occurrence of fluorescence effects, non-radiative relaxation and/or the release of heat into the environment.

This results in a significant reduction in the photodynamic efficiency, i.e. in a reduction in the reactive oxygen species (ROS) formed by photodynamic processes and/or in the excited molecular oxygen formed by photodynamic processes.

In a preferred embodiment of the photosensitizer dispersion in accordance with the invention, in addition to the at least one photosensitizer, it does not contain any further organic compounds which contain unsaturated groups, for example in the form of double bonds and/or triple bonds, and it also does not contain any other organic compounds which comprise oxidizable groups, for example in the form of thiol groups and/or aldehyde groups, because these residues or groups react with singlet oxygen or the reactive oxygen species formed and can reduce the quantum yield.

As an example, in addition to the at least one photosensitizer, the photosensitizer dispersion in accordance with the invention does not contain any further oxidizable aromatic compounds such as phenols, polyphenols, aniline or phenylenediamines, as well as no further activated amino acids such as histidine or tryptophan, no imidazole, no alkyl sulphides and no thioethers.

Preferably, the photosensitizer dispersion in accordance with the invention does not contain any pesticides.

The term “photosensitizer” as used in the context of the invention should be understood to mean compounds which absorb electromagnetic radiation, preferably visible light, UV light and/or infrared light, and thus produce reactive oxygen species (ROS), preferably free radicals and/or singlet oxygen from triplet oxygen.

The term “photodynamic therapy” as used in the context of the invention should be understood to mean the light-induced inactivation of cells or microorganisms, preferably including viruses, archaea, bacteria, bacterial spores, fungi, fungal spores, protozoa, algae, blood-borne parasites or combinations thereof, on and/or in patients.

The term “photodynamic decontamination” as used in the context of the invention should be understood to mean the light-induced inactivation of microorganisms, preferably including viruses, archaea, bacteria, bacterial spores, fungi, fungal spores, protozoa, algae, blood-borne parasites or combinations thereof, on the surfaces of articles, areas and/or foodstuffs and/or in liquids, in particular water, domestic water supplies, grey water, rainwater, process water, etc.

The term “surface cleaning” as used in the context of the invention should be understood to mean the inactivation of microorganisms which preferably include viruses, archaeae, bacteria, bacterial spores, fungi, fungal spores, protozoa, algae, blood-borne parasites or combinations thereof, on the surfaces of articles, areas and/or foodstuffs. The term “surface cleaning and/or coating” as used in the context of the invention does not include surfaces on a human or animal body such as the skin, for example, and/or in a human or animal body such as the outer, apical side of the epithelium of a hollow organ.

The term “inactivation” as used in the context of the invention should be understood to mean a reduction in the viability or the destruction of a microorganism, preferably its destruction. Light-induced inactivation may, for example be ascertained by a reduction in the number of microorganisms following irradiation of a predefined starting quantity of these microorganisms in the presence of a dispersion in accordance with the invention.

In accordance with the invention, the term “reduction in viability” should be understood to mean that the number of microorganisms is reduced by at least 80.0%, preferably at least 99.0%, preferably at least 99.9%, more preferably by at least 99.99%, more preferably by at least 99.999%, yet more preferably by at least 99.9999%. Most preferably, the number of microorganisms is reduced by more than 99.9% to 100%, preferably by more than 99.99% to 100%.

Preferably, the reduction in the number of microorganisms is given in accordance with Boyce, J. M. and Pittet, D. (“Guidelines for hand hygiene in healthcare settings.

Recommendations of the Healthcare Infection Control Practices Advisory Committee and the HIPAC/SHEA/APIC/IDSA Hand Hygiene Task Force”, Am. J. Infect. Control 30 (8), 2002, pages 1-46) as a log₁₀ reduction factor.

In accordance with the invention, the term “log₁₀ reduction factor” should be understood to mean the difference between the logarithm to base 10 of the number of microorganisms before and the logarithm to base 10 of the number of microorganisms following irradiation of these microorganisms with electromagnetic radiation in the presence of a dispersion in accordance with the invention.

Examples of suitable methods for determining the log₁₀ reduction factors are described in DIN EN 14885:2007-01 “Chemical disinfectants and antiseptics. Application of European standards for chemical disinfectants and antiseptics” or in Rabenau, H. F. and Schwebke, I. (“Guidelines from the German Association for the Control of Viral Diseases (DVV) and the Robert Koch Institute (RKI) for testing chemical disinfectants for effectiveness against viruses in human medicine” Bundesgesundheitsblatt, Gesundheitsforschung, Gesundheitsschutz 51(8), (2008), pages 937-945).

Preferably, the log₁₀ reduction factor following the irradiation of microorganisms with electromagnetic radiation in the presence of a dispersion in accordance with the invention is at least 2 log₁₀, preferably at least 3 log₁₀, more preferably at least 4 log₁₀, more preferably at least 4.5 log₁₀, more preferably at least 5 log₁₀, more preferably at least 6 log₁₀, yet more preferably at least 7 logᵢₙ, yet more preferably at least 7.5 log₁₀.

As an example, a “reduction in the number of microorganisms following the irradiation of these microorganisms with electromagnetic radiation in the presence of a dispersion in accordance with the invention by 2 powers of ten with respect to the starting quantity of said microorganisms” means a log₁₀ reduction factor of 2 log₁₀.

More preferably, the number of microorganisms following irradiation of these microorganisms with electromagnetic radiation in the presence of a dispersion in accordance with the invention is reduced by at least 1 power of ten, more preferably by at least 2 powers of ten, more preferably by at least 3 powers of ten, preferably by at least 4 powers of ten, more preferably by at least 5 powers of ten, more preferably by at least 6 powers of ten, yet more preferably by at least 7 powers of ten, respectively with respect to the starting quantity of said microorganisms.

The term “microorganisms” as used in the context of the invention should in particular be understood to refer to viruses, archaea, prokaryotic microorganisms such as fungi, protozoa, fungal spores, or single-celled algae. The microorganisms in this case may be single-celled or multi-celled, for example fungal mycelium.

A photosensitizer dispersion in accordance with the invention comprises (a) at least one photosensitizer.

In a preferred embodiment, the at least one photosensitizer is positively charged, negatively charged, uncharged, or a mixture thereof. More preferably, the at least one photosensitizer comprises at least one organic residue with a) at least one neutral nitrogen atom which can be protonated, and/or b) at least one positively charged nitrogen atom.

In a preferred embodiment, the at least one photosensitizer is selected from the group which consists of phenalenones, curcumins, flavins, porphyrins, porphycenes, xanthene dyes, coumarins, phthalocyanines, phenothiazine compounds, anthracene dyes, pyrenes, fullerenes, perylenes and mixtures thereof, preferably from phenalenones, curcumins, flavins, porphyrins, phthalocyanines, phenothiazine compounds and mixtures thereof, more preferably from phenalenones, curcumins, flavins and mixtures thereof.

Suitable phenalenones are disclosed, for example, in EP 2 678 035 A2, the content of which as regards the structure and synthesis of suitable phenalenones is hereby incorporated by reference.

Preferably, a suitable phenalenone derivative is selected from the group which consists of the compounds with formulae (2) bis (25) and mixtures thereof:

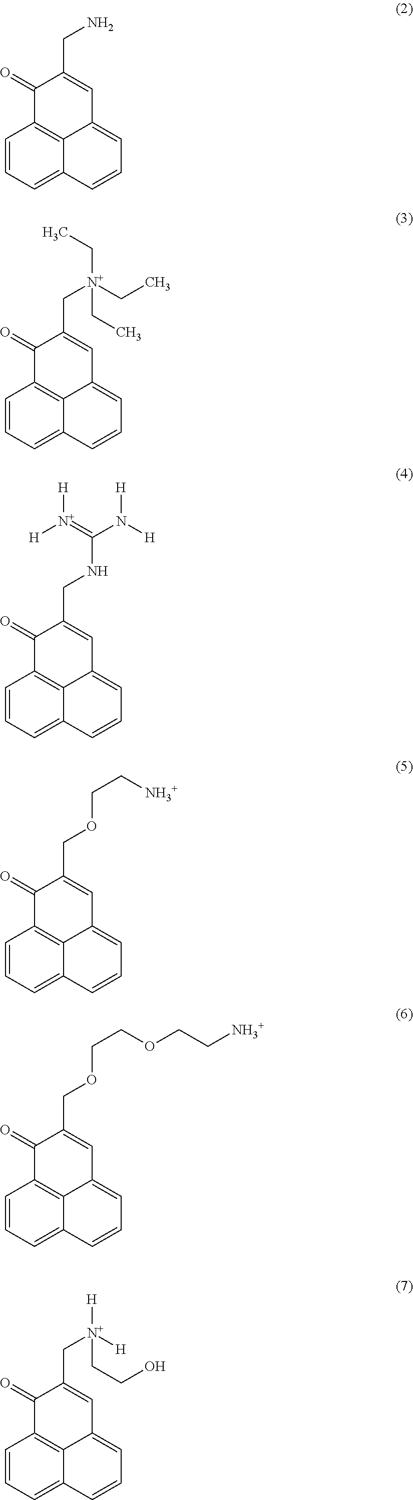 [SMILES: NCC1=Cc2cccc3cccc(c23)C1=O ; CC[N+](CC)(CC)CC1=Cc2cccc3cccc(c23)C1=O ; NC(=[NH2+])NCC1=Cc2cccc3cccc(c23)C1=O ; [NH3+]CCOCC1=Cc2cccc3cccc(c23)C1=O ; [NH3+]CCOCCOCC1=Cc2cccc3cccc(c23)C1=O ; O=C1C(C[NH2+]CCO)=Cc2cccc3cccc1c23]

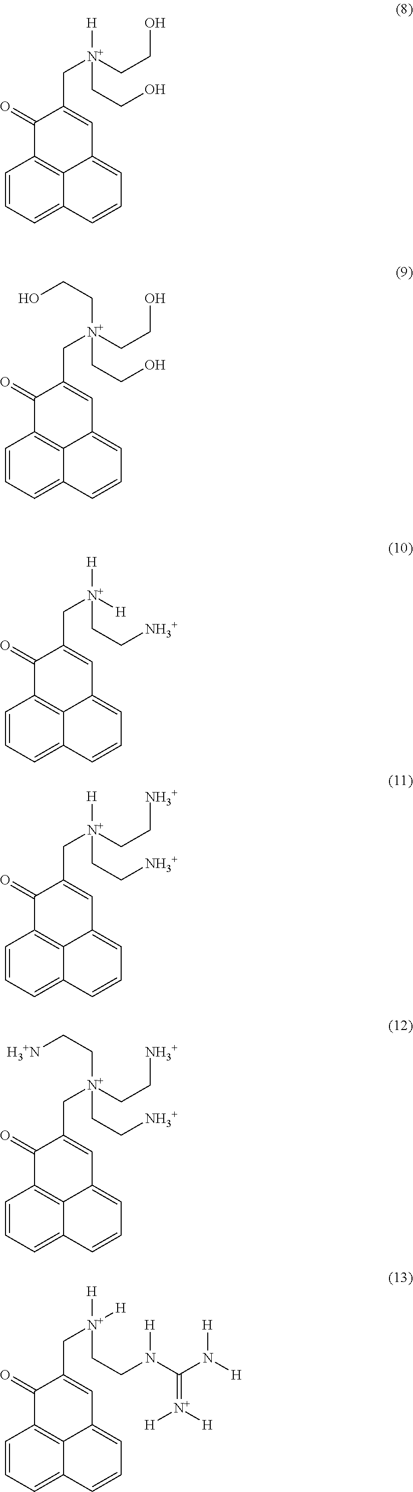

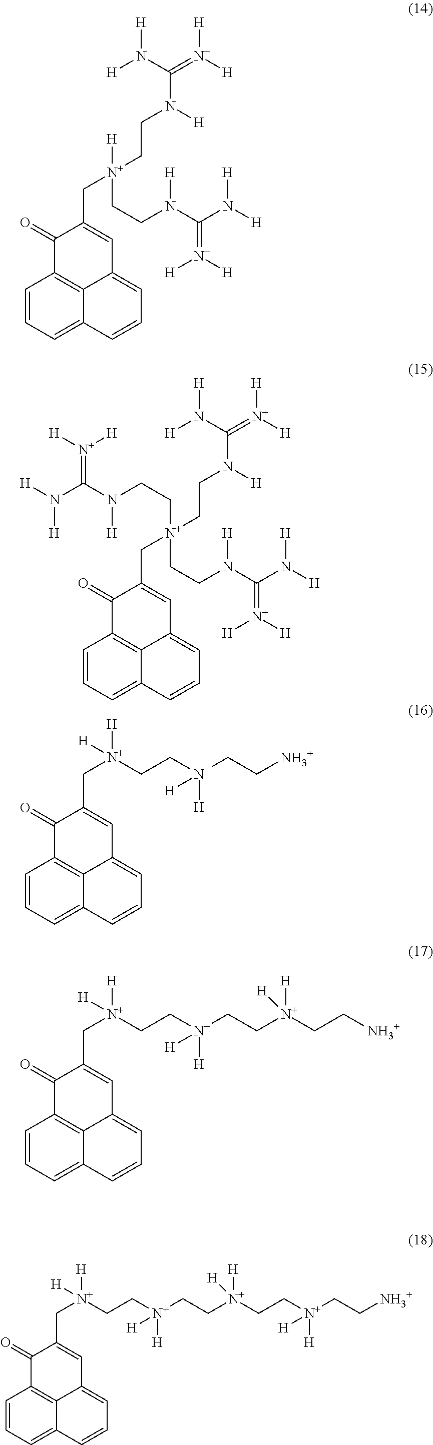 [SMILES: NC(=[NH2+])NCC[NH+](CCNC(N)=[NH2+])CC1=Cc2cccc3cccc(c23)C1=O ; NC(=[NH2+])NCC[N+](CCNC(N)=[NH2+])(CCNC(N)=[NH2+])CC1=Cc2cccc3cccc(c23)C1=O ; [NH3+]CC[NH2+]CC[NH2+]CC1=Cc2cccc3cccc(c23)C1=O ; [NH3+]CC[NH2+]CC[NH2+]CC[NH2+]CC1=Cc2cccc3cccc(c23)C1=O ; [NH3+]CC[NH2+]CC[NH2+]CC[NH2+]CC[NH2+]CC1=Cc2cccc3cccc(c23)C1=O]

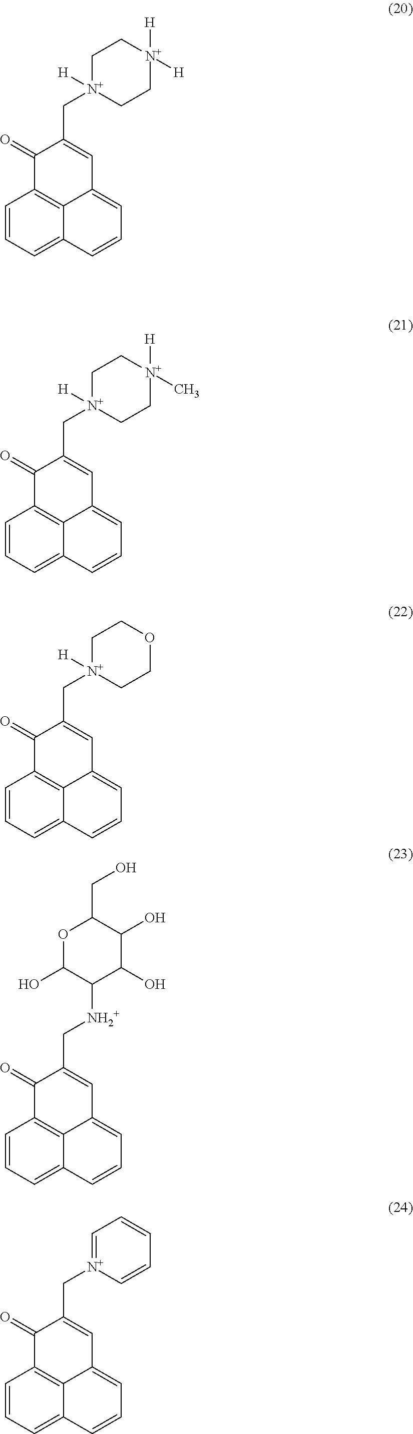 [SMILES: O=C1C(C[NH+]2CC[NH2+]CC2)=Cc2cccc3cccc1c23 ; C[NH+]1CC[NH+](CC2=Cc3cccc4cccc(c34)C2=O)CC1 ; O=C1C(C[NH+]2CCOCC2)=Cc2cccc3cccc1c23 ; O=C1C(C[NH2+]C2C(O)OC(CO)C(O)C2O)=Cc2cccc3cccc1c23 ; O=C1C(C[n+]2ccccc2)=Cc2cccc3cccc1c23]

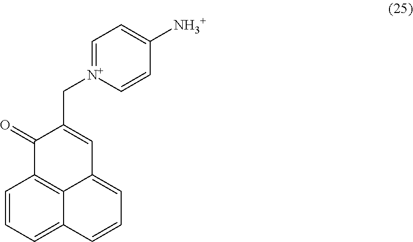 [SMILES: [NH3+]c1cc[n+](CC2=Cc3cccc4cccc(c34)C2=O)cc1]

Preferably, a suitable phenalenone derivative is furthermore selected from the group which consists of the compounds with formulae (26) to (28) and mixtures thereof:

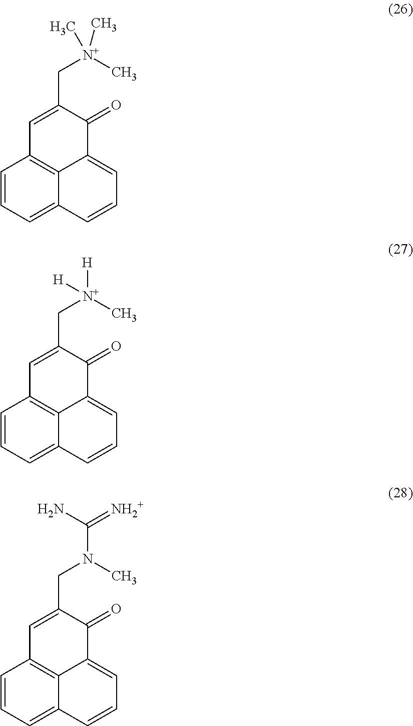 [SMILES: C[N+](C)(C)CC1=Cc2cccc3cccc(c23)C1=O ; C[NH2+]CC1=Cc2cccc3cccc(c23)C1=O ; CN(CC1=Cc2cccc3cccc(c23)C1=O)C(N)=[NH2+]]

More preferably, a suitable phenalenone derivative is selected from the group which consists of the compounds with formulae (2) to (28) and mixtures thereof.

Suitable flavins are disclosed, for example, in EP 2 723 342 A1, EP 2 723 743 A1 and EP 2 723 742 A1, the content of which as regards the structure and synthesis of suitable flavins is hereby incorporated by reference.

Preferably, a suitable flavin derivative is selected from the group which consists of the compounds with formulae (32) to (49), (51) to (64) and mixtures thereof:

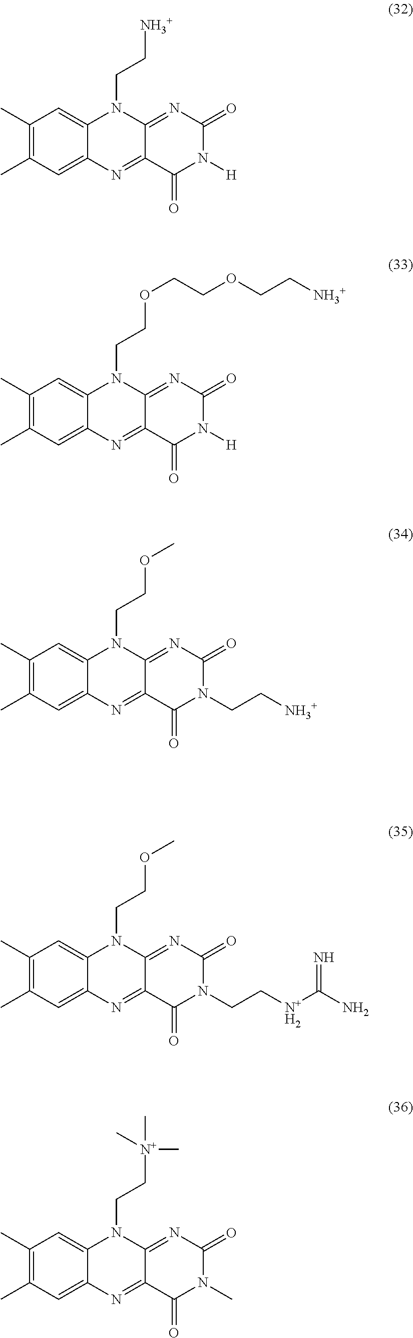

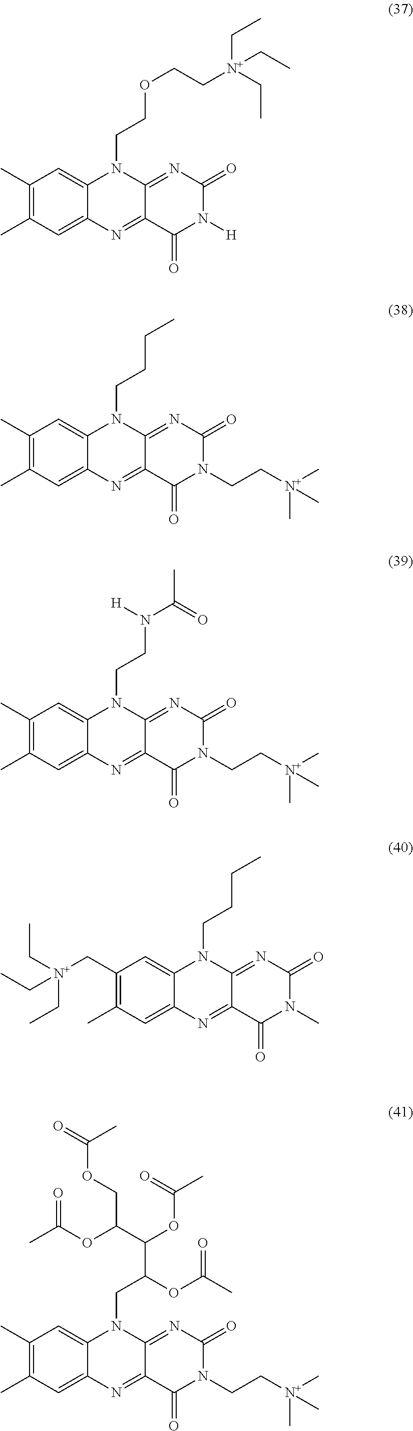

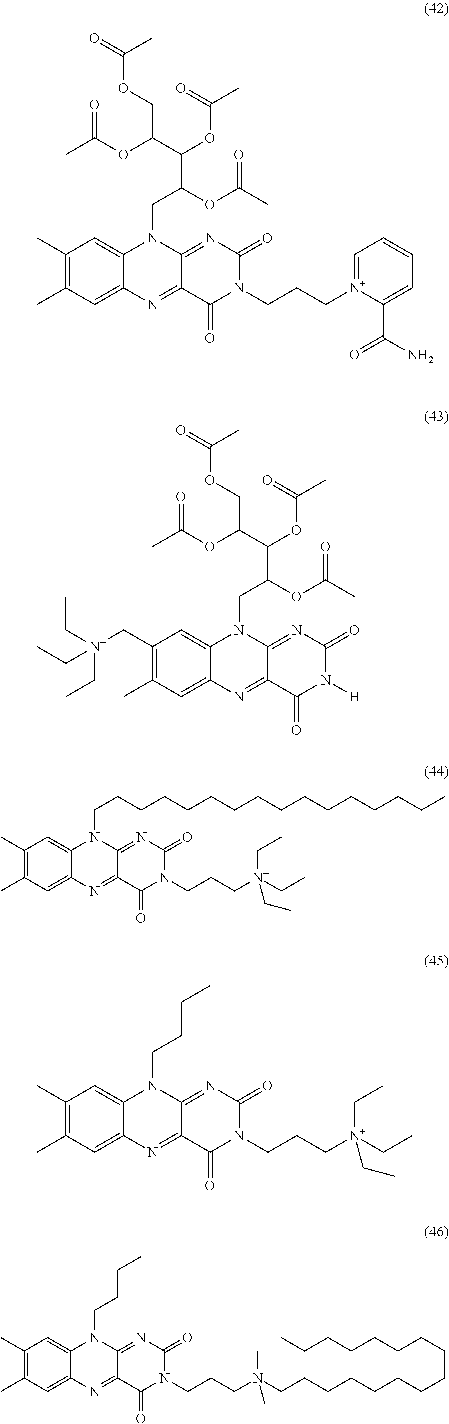

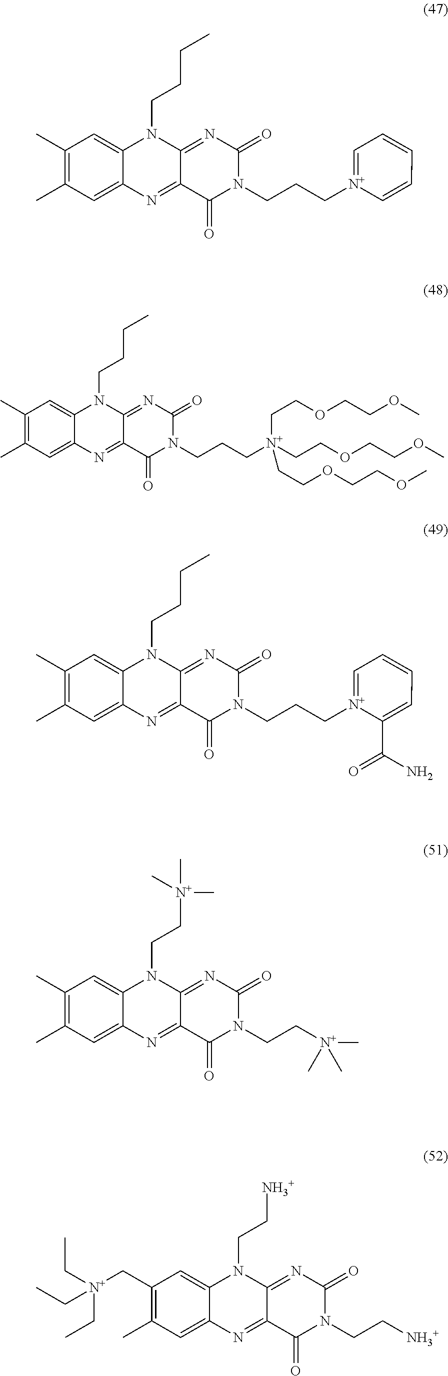

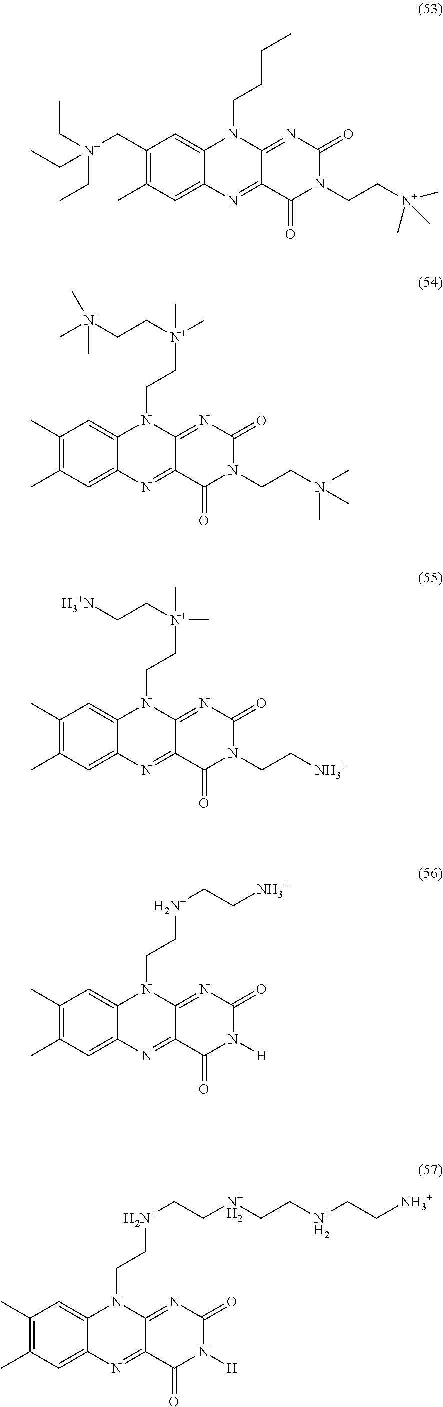

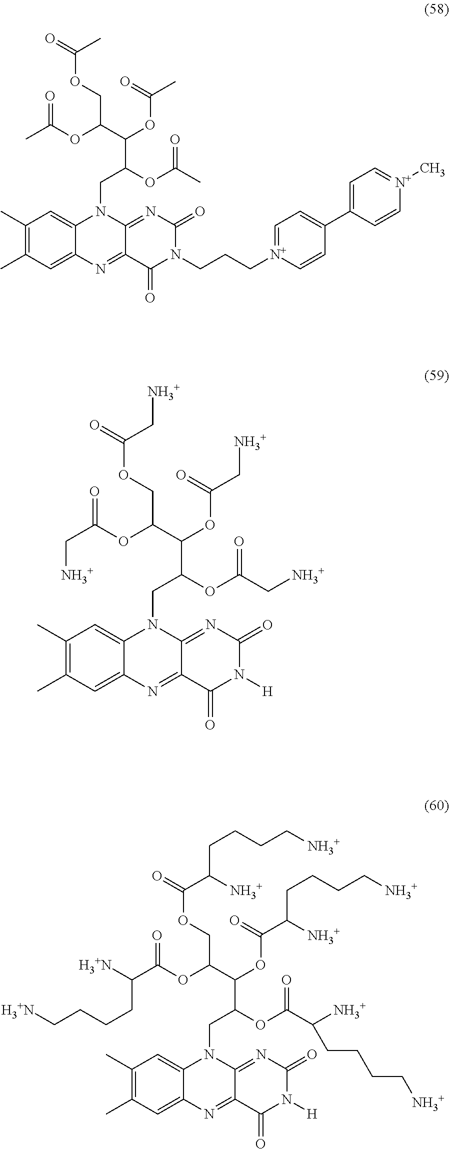

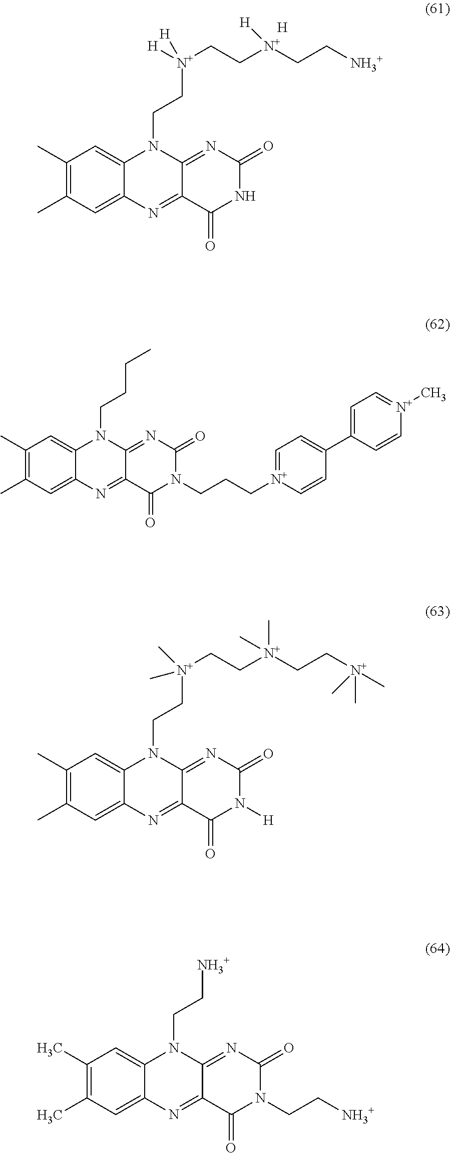

Suitable curcumins are disclosed, for example, in the unpublished patent application EP 18152597.3, the content of which as regards the structure and synthesis of suitable curcumins is hereby incorporated by reference.

A suitable curcumin derivative is selected, for example, from the group which consists of the compounds with formulae (75) to (104b), (105) and mixtures thereof:

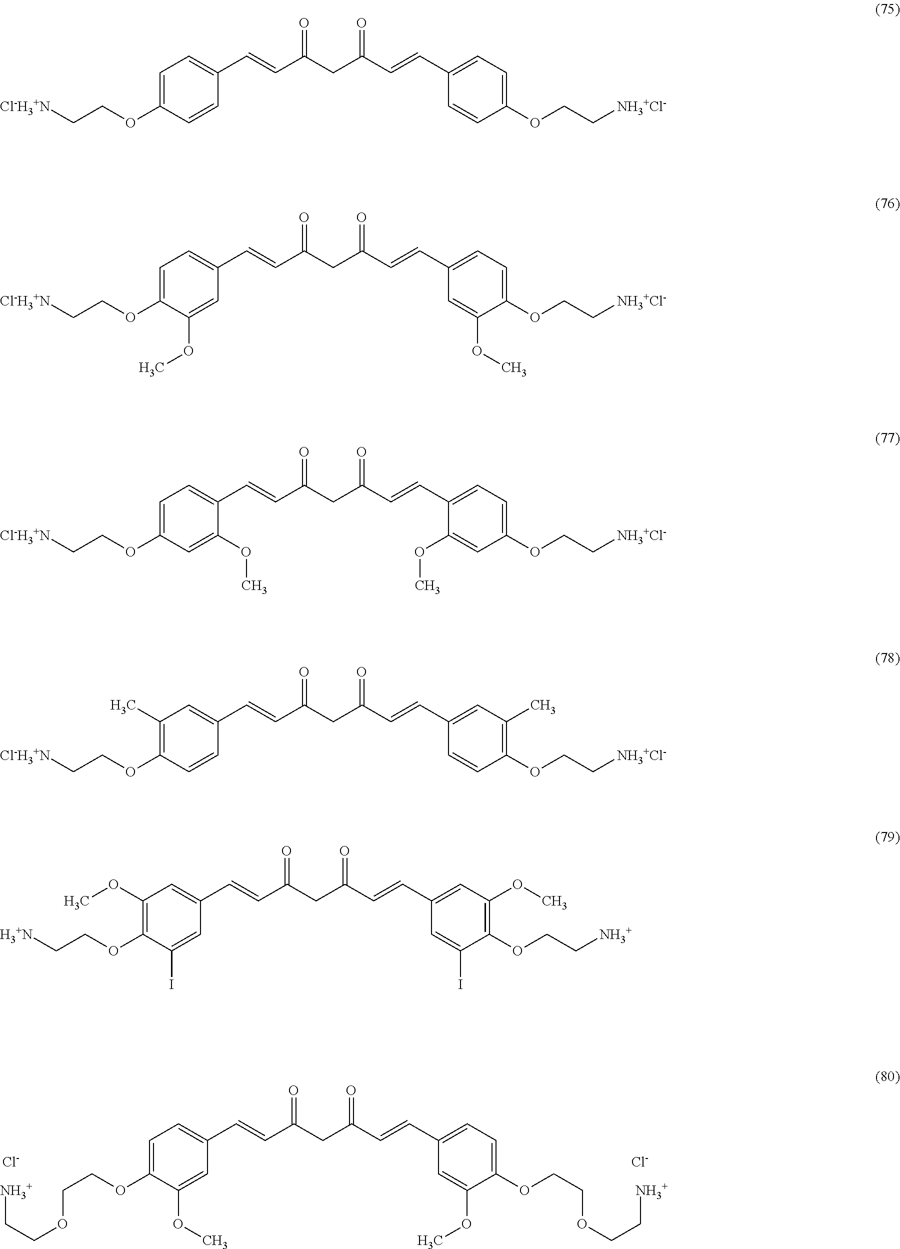

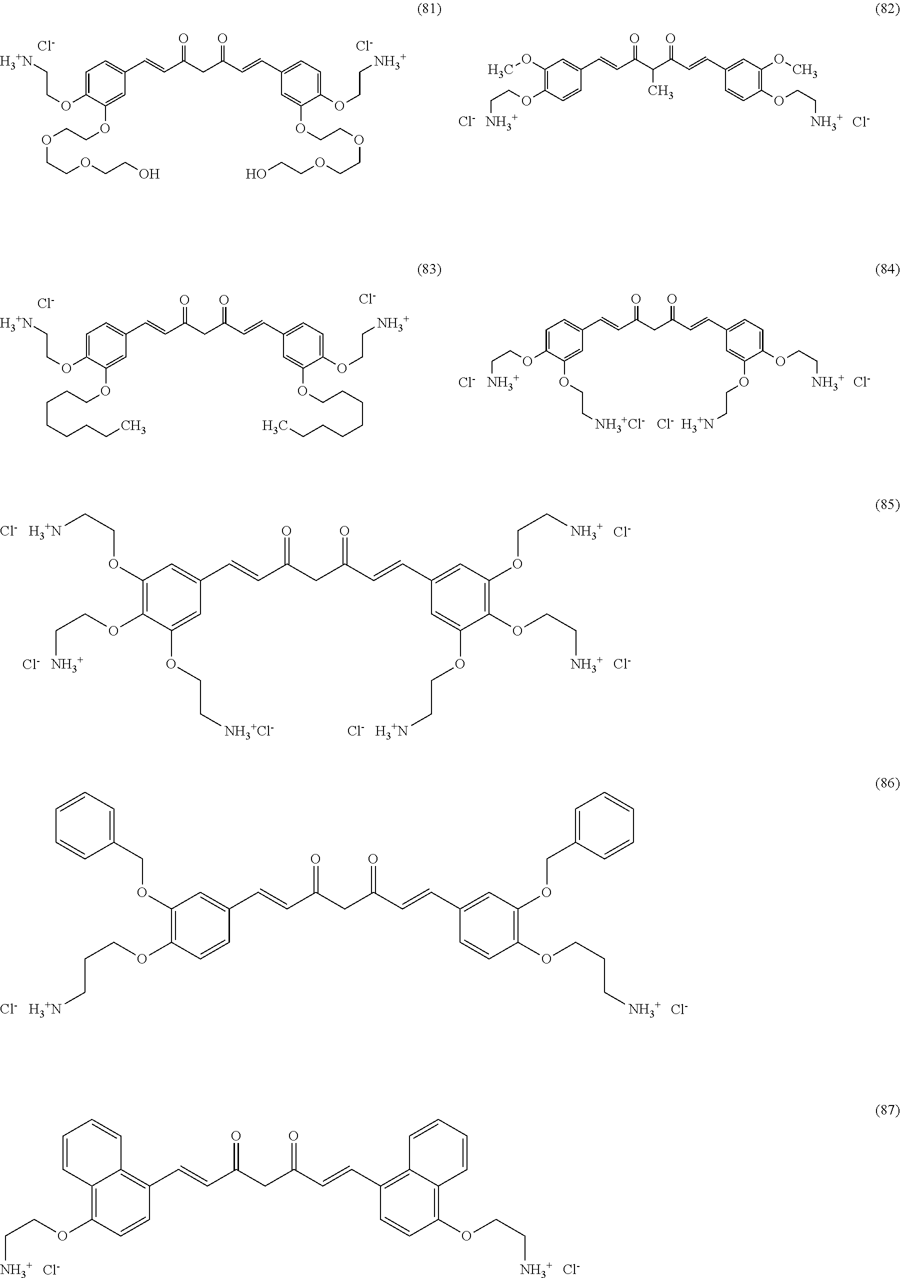

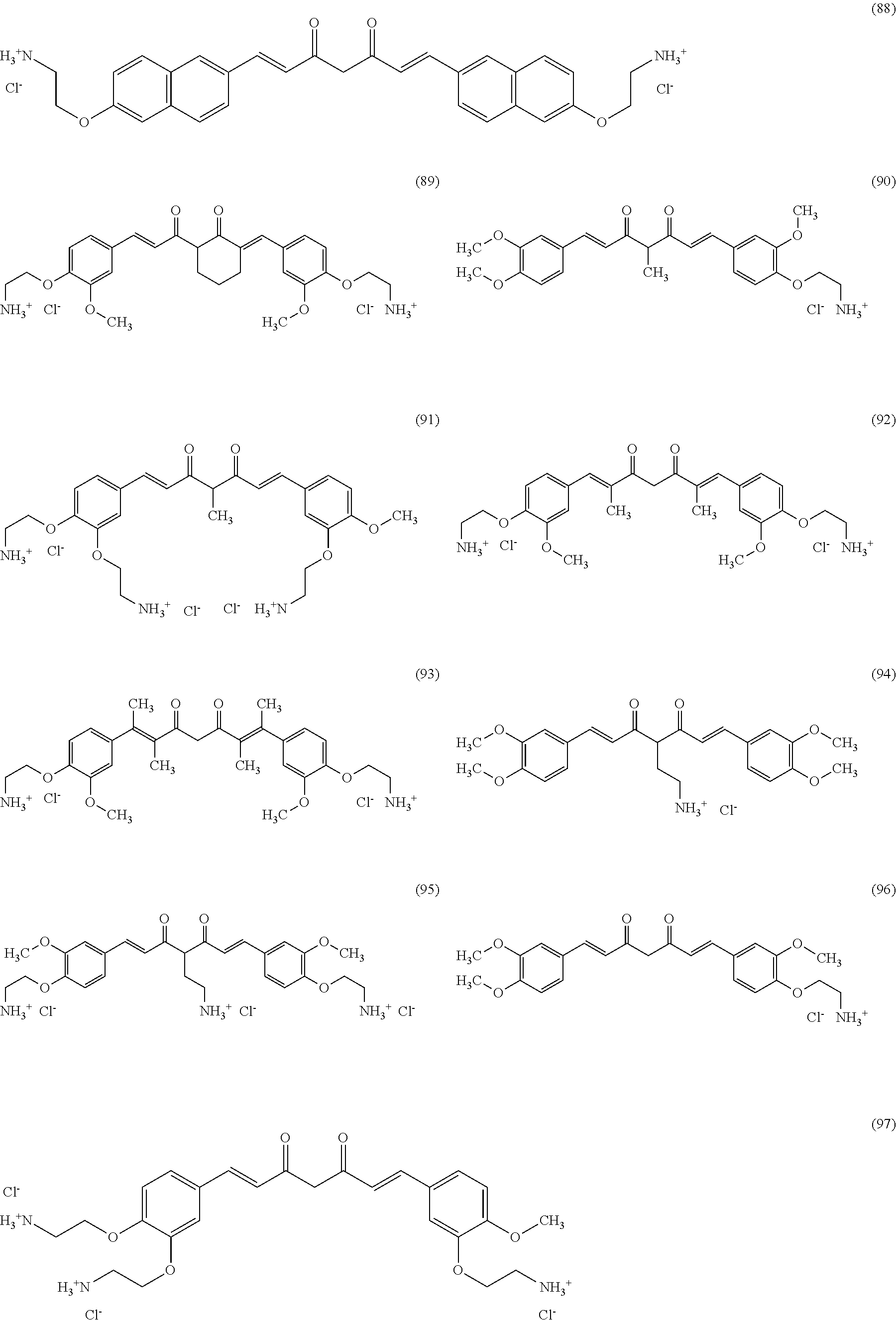

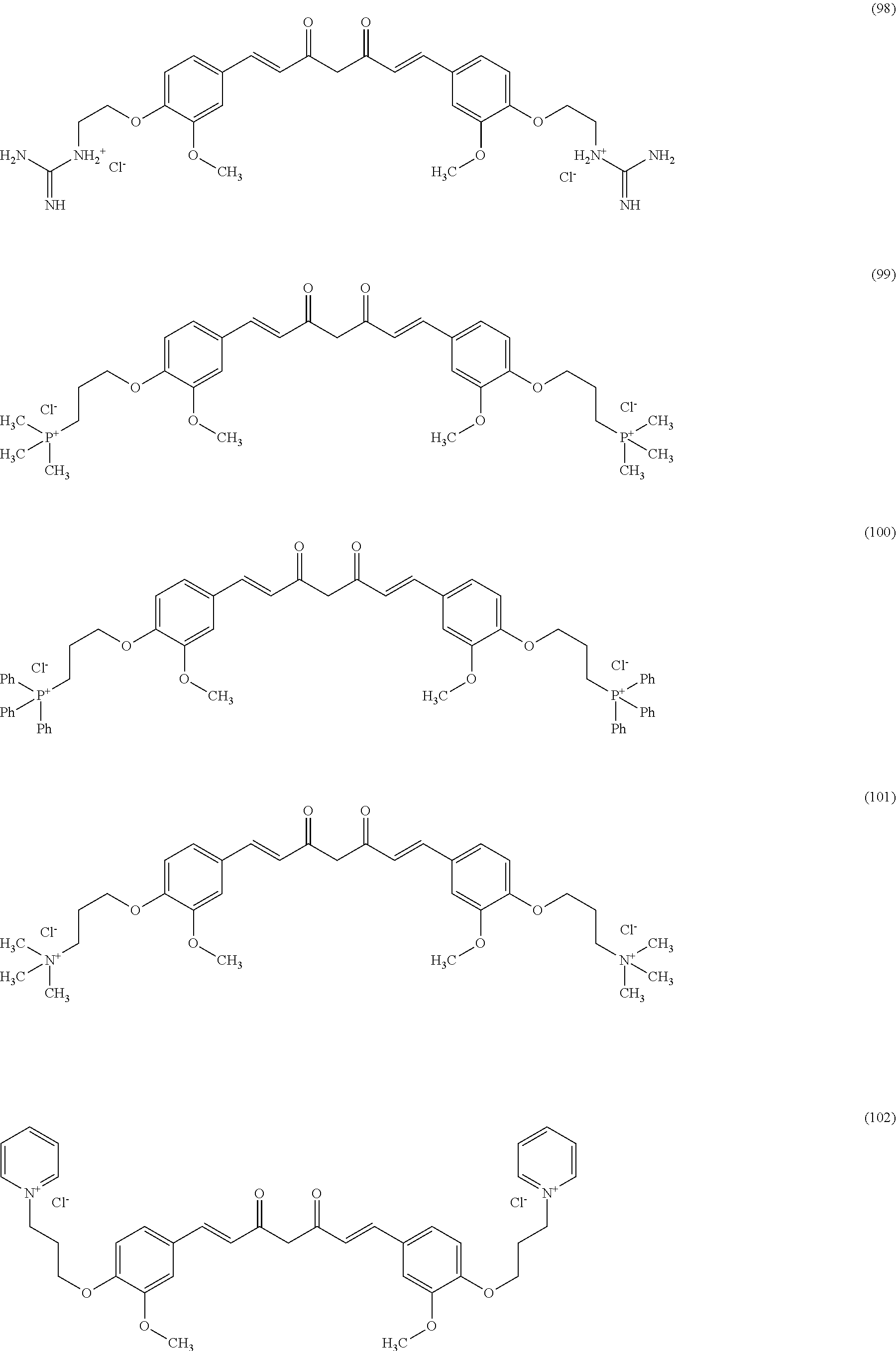

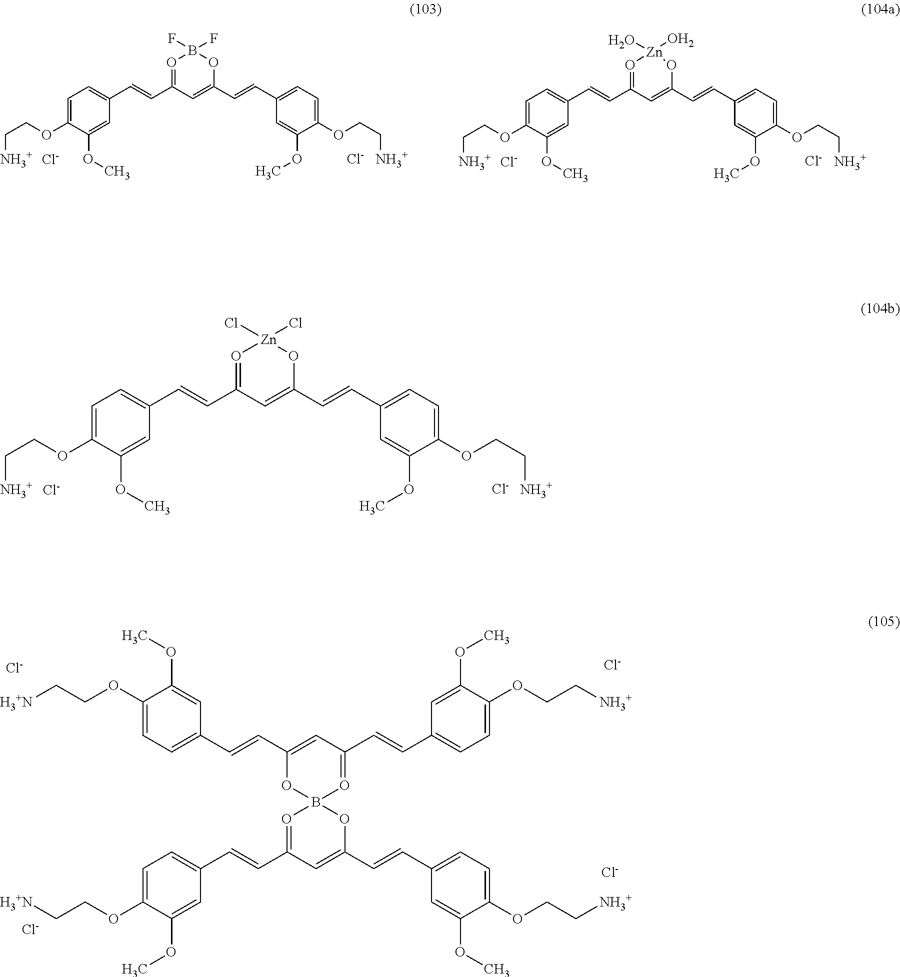

Suitable curcumin derivatives and their manufacture are described, for example, in CA 2 888 140 A1, the content of which as regards the structure and synthesis of suitable curcumins is hereby incorporated by reference.

Suitable curcumin-3,5-dione derivatives and their manufacture are similarly described in EP 2 698 368 A1, the content of which as regards the structure and synthesis of suitable curcumins is hereby incorporated by reference.

A suitable curcumin derivative and its manufacture is similarly described by Taka et al. (Bioorg. Med. Chem. Lett. 24, 2014, pages 5242 bis 5246), the content of which as regards the structure and synthesis of suitable curcumins is hereby incorporated by reference.

Examples of suitable commercially available phenothiazinium dyes are new methylene blue (NMB; 3,7-bis(ethylamino)-2,8-dimethylphenothiazin-5-ium chloride), 1,9-dimethyl methylene blue (DMMB; 3,7-bis-(dimethylamino)-1,9-dimethyl-diphenothiazin-5-ium zinc chloride) or methylene green (basic green 5, [7-(dimethylamino)-4-nitrophenothiazin-3-ylidene]-dimethylazanium chloride).

Examples of suitable commercially available polymethine dyes are cyanine-5 (Cy5), cyanine-3 (Cy3) or indocyanine green (ICG).

Examples of suitable commercially available xanthene dyes are pyronine G, eosine B, eosine Y, Rose Bengal, erythrosine (E127) or phloxine B,

Examples of suitable commercially available triphenylmethane dyes are Patent Blue V (4-[4,4′-bis(diethylamino)-α-hydroxy-benzhydryl]-6-hydroxy benzene-1,3-disulphonic acid), malachite green (N,N,N′,N′-tetramethyl-4,4′-diaminotriphenylcarbenium chloride), magenta (4-[(4-aminophenyl)-(4-imino-1-cyclohexa-2,5-dienylidene)methyl]aniline hydrochloride), pararosaniline (4,4′-(4-iminocyclohexa-2,5-dienylidenmethylene)dianiline hydrochloride), crystal violet ((4-(4,4′-bis(dimethylaminophenyl)benzhydrylidene)cyclohexa-2,5-dien-1-ylidene)dimethylammonium chloride).

Examples of suitable commercially available anthraquinone dyes are (1,2-dihydroxyanthraquinone) or indanthrene (6,15-dihydro-5,9,14,18-anthracene tetrone).

Examples of suitable commercially available porphyrin dyes are 5,10,15,20-tetrakis(1-methyl-4-pyridinio)porphyrin-tetra(p-toluenesulphonate) (TMPyP), or tetrakis(p-trimethylammoniumphenyl)porphyrin chloride.

Examples of suitable commercially available phthalocyanine dyes are zinc phthalocyanine tetrasulphonate or tetrakis(p-trimethylammonium)phthalocyanine zinc chloride,

Examples of suitable commercially available indamine dyes are safranin T (3,7-diamino-2,8-dimethyl-5-phenylphenazinium chloride) or phenosafranine (3,7-diamino-5-phenylphenazinium chloride).

Examples of commercial sources of the dyes mentioned above are AppliChem GmbH (Darmstadt, DE), Frontier Scientific Inc. (Logan, Utah, USA), GE Healthcare Europe GmbH (Freiburg, DE), Sigma-Aldrich Corporation (St. Louis, Mo., USA) or Merck KGaA (Darmstadt, DE).

Any suitable anion may be used as a counterion to the positively charged nitrogen atom. Preferably, anions are used as the counterion to the positively charged nitrogen atom which are selected from the group which consists of fluoride, chloride, bromide, iodide, sulphate, hydrogen sulphate, phosphate, dihydrogen phosphate, hydrogen phosphate, tosylate, mesylate, formate, acetate, propionate, butanoate, oxalate, tartrate, fumarate, benzoate, citrate and/or mixtures thereof.

Preferably, the at least one photosensitizer is selected from the group which consists of the compounds with formulae (2) to (25), (32) to (49), (51) to (64), (75) to (105) and mixtures thereof.

Preferably, the dispersion comprises the at least one photosensitizer in a concentration in the range 0.1 μM to 1000 μM.

A dispersion in accordance with the invention further comprises (b) at least one liquid polar phase.

Preferably, the at least one liquid polar phase is in the liquid physical state at a temperature in the range 0° C. to 100° C. and a pressure in the range 800 to 1200 mbar.

Preferably, the at least one liquid polar phase comprises at least one polar solvent, preferably water.

Preferably, a dispersion in accordance with the invention comprises the at least one polar solvent, preferably water, in a proportion of at least 0.1% by weight, preferably at least 0.5% by weight, more preferably at least 1% by weight, more preferably at least 4% by weight, more preferably at least 10% by weight, more preferably at least 35% by weight, more preferably at least 50% by weight, more preferably at least 51% by weight, respectively with respect to the total weight of the dispersion.

Preferably, a dispersion in accordance with the invention comprises the at least one polar solvent, preferably water, in a proportion in the range 0.1% by weight to 99.8% by weight, preferably in the range 0.5% by weight to 99% by weight, more preferably in the range 4% by weight to 98% by weight, more preferably in the range 10% by weight to 97% by weight, more preferably in the range 35% by weight to 96% by weight, more preferably in the range 50% by weight to 95% by weight, more preferably in the range 51% by weight to 94% by weight, more preferably in the range 53% by weight to 93% by weight, more preferably in the range 70% by weight to 92% by weight, respectively with respect to the total weight of the dispersion.

A dispersion in accordance with the invention further comprises (c) at least one surfactant.

Preferably, a dispersion in accordance with the invention comprises the at least one surfactant in a proportion in the range 0.1% by weight to 65% by weight, preferably in the range 1% by weight to 55% by weight, more preferably in the range 3% by weight to 50% by weight, more preferably in the range 5% by weight to 41% by weight, more preferably in the range 7% by weight to 37% by weight, more preferably in the range 9% by weight to 30% by weight, more preferably in the range 10% by weight to 27% by weight, respectively with respect to the total weight of the dispersion.

The at least one surfactant is preferably selected from the group which consists of non-ionic surfactants, anionic surfactants, cationic surfactants, amphoteric surfactants and mixtures thereof, preferably non-ionic surfactants, anionic surfactants and mixtures thereof.

The at least one surfactant preferably has an HLB value in the range 4 to 40, preferably in the range 5 to 20. The HLB value of a surfactant may, for example, be determined in accordance with the methods described in Griffin, W. C. (1949) (“Classification of Surface-Active Agents by ‘HLB’”, J. Soc. Cosmet. Chem. 1 (5), pages 311 to 326) or in Griffin, W. C. (1954) (“Calculation of HLB Values of Non-Ionic Surfactants”, J. Soc. Cosmet. Chem. 5 (4): pages 249 to 256).

Preferably, suitable non-ionic surfactants are selected from the group which consists of polyalkyleneglycol ethers, alkylglucosides, al kylpolyglycosides, alkylglycoside esters, and mixtures thereof.

Suitable polyalkyleneglycol ethers preferably have the general formula (I):

CH₃—(CH₂)ₘ—(O—[CH₂]ₓ)ₙ—OH, (I)

wherein m=8-20, preferably 10-16, wherein n=1-25, wherein x=1, 2, 3 or 4.

Preferably, a combination of different polyalkyleneglycol ethers is used, for example with different alkyloxy units (—(O—[CH₂]ₓ)ₙ—).

Examples of suitable polyalkyleneglycol ethers are polyoxyethylene ethers of lauryl alcohol (dodecan-1-ol), polyoxyethylene ethers of cetyl alcohol (hexadecan-1-ol), polyoxyethylene ethers of stearyl alcohol (1-octadecanol), polyoxyethylene ethers of oleyl alcohol ((E)-octadec-9-en-1-ol) or polyoxyethylene ethers of mixture of stearyl alcohol and of cetyl alcohol, (cetylstearyl alcohol).

Suitable polyalkyleneglycol ethers are commercially available under the trade names: Brij, Thesit, Cremophor, Genapol, Magrogol, Lutensol etc, for example.

Examples of suitable polyalkyleneglycol ethers are:

| Chemical name | INCI name | Trade name |
|---|---|---|
| Polyoxyethylene (4) | Laureth-4 (INCI) | Brij ® 30 |
| lauryl ether | | |
| Polyoxyethylene (9) | Laureth-9 (INCI) | Thesit ® |
| lauryl ether | | |
| Polyoxyethylene (23) | Laureth-23 (INCI) | Brij ® 35 |
| lauryl ether | | |
| Polyoxyethylene (2) | Ceteth-2 (INCI) | Brij ® 52 |
| cetyl ether | | |
| Polyoxyethylene (10) | Ceteth-10 (INCI) | Brij ® 56 |
| cetyl ether | | |
| Polyoxyethylene (20) | Ceteth-20 (INCI) | Brij ® 58 |
| cetyl ether | | |
| Polyoxyethylene (6) | Ceteareth-6 (INCI) | Cremophor A6 |
| cetylstearyl ether | | |
| Polyoxyethylene (20) | Ceteareth-20 (INCI) | |
| cetylstearyl ether | | |
| Polyoxyethylene (25) | Ceteareth-25 (INCI) | Cremophor A25, |
| cetylstearyl ether | | |
| Polyoxyethylene (2) | Steareth-2 (INCI) | Brij ® 72 |
| stearyl ether | | |
| Polyoxyethylene (10) | Steareth -10 (INCI) | Brij ® 76 |
| stearyl ether | | |
| Polyoxyethylene (20) | Steareth -20 (INCI) | Brij ® 78 |
| stearyl ether | | |
| Polyoxyethylene (2) | Oleth-2 (INCI) | Brij ® 92 |
| oleyl ether | | |
| Polyoxyethylene (10) | Oleth -10 (INCI) | Brij ® 96 |
| oleyl ether | | |
| Polyoxyethylene (20) | Oleth -20 (INCI) | Brij ® 98 |
| oleyl ether | | |

Examples of suitable alkylglucosides are ethoxylated sorbitan fatty acid esters (polysorbates), which are commercially available, for example, under the trade name Tween® from Croda International Plc (Snaith, UK).

| Chemical name | INCI name | Trade name |
|---|---|---|
| Polyoxyethylene-(20)-sorbitan | Polysorbate 20 | Tween ® 20 |
| monolaurate | | |
| Polyoxyethylene-(4)-sorbitan | Polysorbate 21 | Tween ® 21 |
| monolaurate | | |
| Polyoxyethylene-(20)-sorbitan | Polysorbate 40 | Tween ® 40 |
| monopalmitate | | |
| Polyoxyethylene-(20)-sorbitan | Polysorbate 60 | Tween ® 60 |
| monostearate | | |
| Polyoxyethylene-(4)-sorbitan | Polysorbate 61 | Tween ® 61 |
| monostearate | | |
| Polyoxyethylene-(20)- | Polysorbate 65 | Tween ® 65 |
| sorbitantristearate | | |
| Polyoxyethylene-(20)- | Polysorbate 80 | Tween ® 80 |
| sorbitan monooleate | | |
| Polyoxyethylene-(5)- | Polysorbate 81 | Tween ® 81 |
| sorbitan monooleate | | |
| Polyoxyethylene-(20)- | Polysorbate 85 | Tween ® 85 |
| sorbitantrioleate | | |
| Polyoxyethylene-(20)-sorbitan | Polysorbate 120 | |
| monoisostearate | | |

An example of a further suitable alkylglucoside is the surfactant Kosteran SQ/O VH, which is commercially available from Dr. W. Kolb AG (Hedingen, CH). Kosteran SQ/O VH is a sorbitan oleic acid ester with an average of 1.5 oleic acid molecules per molecule (sorbitan sesquioleate).

An example of a further suitable alkylglucoside is PEG-80 sorbitan laurate, an ethoxylated sorbitan monoester of lauric acid with an average ethylene oxide content of 80 Mol ethylene oxide per molecule. PEG-80 sorbitan laurate is commercially available from Croda International Plc under the trade name Tween® 28.

Suitable alkylglycoside esters are fatty acid esters of methyl or ethyl glycosides, for example methylglycoside esters and ethylglycoside esters, or saccharose esters.

Preferably, suitable anionic surfactants are selected from the group which consists of alkylcarboxylates, alkylsulphonates, alkylsulphates, alkylphosphates, alkylpolyglycolether sulphates, sulphonates of alkylcarboxylic acid esters, N-alkyl-sarcosinates, and mixtures thereof.

Suitable alkylcarboxylates preferably have the general formula (II):

H₃C—(CH₃)ₐ—CH₂—COO⁻M⁺, (II)

wherein a=5-21, preferably 8-16, and wherein M⁺ is a water-soluble cation, preferably a cation of an alkali metal or ammonium, preferably Li⁺, Na⁺, K⁺ or NH₄⁺.

Suitable alkylsulphonates preferably contain 3-30 C atoms. Preferred suitable alkylsulphonates are monoalkylsulphonates containing 8-20 C atoms, secondary alkylsulphonates with general formula (III):

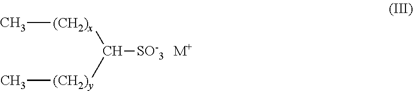

wherein x, y respectively independently of each other=0-17, wherein preferably, x+y=10 to 20, and wherein M⁺ represents a water-soluble cation, preferably a cation of an alkali metal or ammonium, preferably Li⁺, Na⁺, K⁺ or NH₄⁺.

Suitable alkylsulphates preferably have the general formula (IV):

H₃C—(CH₃)_{d}—CH₂—O—SO₃⁻M⁺, (IV)

wherein d=6-20, preferably 8-18, and wherein M⁺ represents a water-soluble cation, preferably a cation of an alkali metal or ammonium, preferably Li⁺, Na⁺, K⁺ or NH₄⁺.

An example of a suitable alkylsulphate is sodium dodecylsulphate (SDS).

Suitable alkylphosphates preferably have the general formula (V):

H₃C—(CH₃)ₑ—CH₂—O—PO₃²⁻2×M⁺, (V)

wherein e=6-20, preferably 8-18 and wherein M⁺ represents a water-soluble cation, preferably a cation of an alkali metal or ammonium, preferably Li⁺, Na⁺, K⁺ or NH₄⁺.

Preferred suitable alkylpolyglycolethersulphates have an alkyl residue containing 6 to 22 carbon atoms, preferably 8 to 20 carbon atoms, and 1 to 10 ethylene oxide units, preferably 2 to 6 ethylene oxide units, in the ether portion.

An example of a suitable N-alkyl sarcosinate is N-lauroyl sarcosinate.

Preferred suitable sulphonates of alkylcarboxylic acid esters contain 6 to 30 carbon atoms, preferably 8 to 20 carbon atoms.

Preferably, suitable sulphonates of alkylcarboxylic acid esters comprise at least one alkyl residue containing 6 to 20 carbon atoms, preferably 8 to 18 carbon atoms, and an alkylcarboxylic acid residue containing 2 to 10 carbon atoms, preferably 2 to 6 carbon atoms. The alkyl residue may contain polyoxyethylene (POE) groups.

Examples of suitable sulphonates of alkylcarboxylic acid esters are monoalkylester sulphosuccinates or dialkylestersulphosuccinates, for example dioctylsodium sulphosuccinate.

Preferably, the suitable cationic surfactants are quaternary alkylammonium salts, esterquats, acylated polyamines, benzylammonium salts or mixtures thereof.

Suitable alkylammonium salts preferably contain the general formula (VI):

(R¹)(R²)(R³)(R⁴)N⁺Z⁻ (VI)

wherein the organic residue R¹ is an alkyl residue, which may be linear or branched, preferably linear, containing 8 to 20 C atoms, preferably 10 to 18 C atoms, more preferably 12 to 16 C atoms, wherein the organic residues R², R³, and R⁴, respectively independently of each other, represent an alkyl residue, which may be linear or branched, preferably linear, containing 1 to 20 C atoms, preferably containing 1 to 16 C atoms, more preferably containing 1 to 12 C atoms, and wherein represents an anion which is preferably selected from the group which consists of fluoride, chloride, bromide, iodide, sulphate, hydrogen sulphate, phosphate, dihydrogen phosphate, hydrogen phosphate, tosylate, mesylate, formate, acetate, propionate, butanoate, oxalate, tartrate, fumarate, benzoate, citrate and/or mixtures thereof.

Preferably, the organic residue R¹ is an alkyl residue which is selected from the group which consists of octyl, nonyl, decyl, undecyl, dodecyl, tridecyl, tetradecyl, pentadecyl, hexadecyl, heptadecyl, octadecyl, nonadecyl, eicosadecyl and combinations thereof, preferably dodecyl, tridecyl, tetradecyl, pentadecyl, hexadecyl and combinations thereof.

Preferably, the organic residues R², R³, and R⁴, respectively independently of each other, are an alkyl residue which is selected from the group which consists of methyl, ethyl, propyl, butyl, octyl, nonyl, decyl, undecyl, dodecyl, tridecyl, tetradecyl, pentadecyl, hexadecyl, heptadecyl, octadecyl, nonadecyl, eicosadecyl and combinations thereof, preferably methyl, ethyl, propyl, butyl, dodecyl, tridecyl, tetradecyl, pentadecyl, hexadecyl and combinations thereof.

An example of a suitable alkylammonium salt with general formula (VI) is a monoalkyltrimethylammonium salt of an anion which is preferably selected from the group which consists of fluoride, chloride, bromide, iodide, sulphate, hydrogen sulphate, phosphate, dihydrogen phosphate, hydrogen phosphate, tosylate, mesylate, formate, acetate, propionate, butanoate, oxalate, tartrate, fumarate, benzoate, citrate and/or mixtures thereof, wherein the organic residue R¹ is an alkyl residue which is selected from the group which consists of octyl, nonyl, decyl, undecyl, dodecyl, tridecyl, tetradecyl, pentadecyl, hexadecyl, heptadecyl, octadecyl, nonadecyl, eicosadecyl and combinations thereof, preferably dodecyl, tridecyl, tetradecyl, pentadecyl, hexadecyl and combinations thereof, and wherein the organic residues R², R³ and R⁴ each represent methyl.

An example of a suitable alkylammonium salt with general formula (VI) is a dialkyltrimethylammonium salt of an anion which is preferably selected from the group which consists of fluoride, chloride, bromide, iodide, sulphate, hydrogen sulphate, phosphate, dihydrogen phosphate, hydrogen phosphate, tosylate, mesylate, formate, acetate, propionate, butanoate, oxalate, tartrate, fumarate, benzoate, citrate and/or mixtures thereof, wherein the organic residue R¹ and R², respectively independently of each other, represents an alkyl residue which is selected from the group which consists of octyl, nonyl, decyl, undecyl, dodecyl, tridecyl, tetradecyl, pentadecyl, hexadecyl, heptadecyl, octadecyl, nonadecyl, eicosadecyl and combinations thereof, preferably dodecyl, tridecyl, tetradecyl, pentadecyl, hexadecyl and combinations thereof, and wherein the organic residues R³ and R⁴ each represent methyl.

Preferred suitable alkylammonium salts with general formula (VI) are dodecyltrimethylammonium bromide (DTAB) and/or didodecyldimethylammonium bromide (DDAB).

Suitable esterquats comprise, for example, triethanolamine diesterquats, diethanolmethylamine diesterquats or mixtures thereof.

Suitable esterquats may, for example, be produced from triethanolamine or diethanolmethylamine wherein, for example, diethanolmethylamine is esterified with one or two molecules of a fatty acid or, in the case of triethanolamine, with one, two or three molecules of a fatty acid, preferably with two molecules of a fatty acid, and then is quaternized with methyl chloride, methyl bromide or with dimethylsulphate. The fatty acids used for esterification are fatty acids containing 8 to 24 carbon atoms, which may be saturated or unsaturated.

Preferably, suitable amphoteric surfactants have both a negative as well as a positively charged functional group. Examples of suitable amphoteric surfactants are alkylbetaines of alkyl residues containing 8-20 C atoms, alkylsulphobetaines of alkyl residues containing 8-20 C atoms, lecithins or combinations thereof.

Examples of suitable amphoteric surfactants are CHAPS (3-[(3-cholamidopropyl)dimethylammonio]-1-propanesulfonate), CHAPSO (3-[(3-cholamidopropyl)dimethylammonio]-2-hydroxy-1-propanesulfonate), cocamidopropylhydroxysultaine, 1,2 di-n-octanoyl-sn-glycero-3-phosphocholine, 1,2-di-O-hexadecyl-sn-glycero-3-phosphocholine or cocamidopropylbetaine.

A dispersion in accordance with the invention preferably further comprises at least one alkanol containing 2 to 12 carbon atoms, and at least 1 OH group, preferably containing 1 to 6 OH groups.

Preferably, a dispersion in accordance with the invention comprises the at least one alkanol in a proportion in the range 0% by weight to 50% by weight, preferably in the range 0.1% by weight to 40% by weight, more preferably in the range 0.5% by weight to 35% by weight, more preferably in the range 1% by weight to 30% by weight, more preferably in the range 1.5% by weight to 25% by weight, more preferably in the range 5% by weight to 20% by weight, more preferably in the range 7% by weight to 19% by weight, more preferably in the range 10% by weight to 17% by weight, respectively with respect to the total weight of the dispersion.

Preferably, when using at least one anionic surfactant, cationic surfactant or amphoteric surfactant, the at least one alkanol containing 2 to 12 carbon atoms is used as a co-surfactant.

Suitable alkanols are alkanols which are branched or unbranched, preferably unbranched, containing 2 to 12 carbon atoms and at least 1 OH group, preferably 1 to 6 OH groups, preferably 1 to 3 OH groups, or mixtures thereof.

Preferred suitable alkanols are branched or unbranched and contain 2 to 12 carbon atoms, more preferably 4 to 10 carbon atoms.

Preferred suitable alkanols containing 1 OH group are selected from the group which consists of ethanol, 1-propanol, 2-propanol, 1-butanol, 2-methyl-2-propanol, 1-pentanol, 3-methyl-1-butanol, 1-hexanol, 1-heptanol, 1-octanol, 1-nonanol, 1-decanol, 1-undecanol, 1-dodecanol, and mixtures thereof.

Suitable unbranched alkanols containing 2 or more OH groups, preferably 2 or 3 OH groups, and are preferably selected from the group which consists of propan-1,2-diol (propyleneglycol) propan-1,3-diol, butan-1,2-diol, butan-1,3-diol, butan-1,4-diol, butan-2,3-diol, pentan-1,5-diol, octan-1,8-diol, propan-1,2,3-triol (glycerin) or mixtures thereof.

Preferably, the weight ratio of surfactants to alkanol is 4:1 to 1:4, preferably 3:1 to 1:3, preferably 2:1 to 1:2, more preferably 1:1.

In the dispersion in accordance with the invention, a component of the dispersion in accordance with the invention is preferably finely divided (dispersed phase) in another continuous component of the dispersion in accordance with the invention (dispersion medium, coherent phase).

Preferably, a dispersion in accordance with the invention, at a temperature in the range 2° C. to 50° C. and a pressure in the range 800 to 1200 mbar, is a thermodynamically stable dispersion which comprises at least one liquid phase and which preferably hardly ever separates, preferably never separates out.

Preferably, a dispersion in accordance with the invention comprises or is a microemulsion, a gel, preferably a lyogel, or a mixture thereof, preferably a microemulsion and/or a lyogel.

The inventors have established that a dispersion in accordance with the invention, which comprises or is a microemulsion, a gel, preferably a lyogel, or a mixture thereof, at a temperature in the range 2° C. to 50° C. and a pressure in the range 800 to 1200 mbar, hardly ever separates, preferably never separates, over a period which is preferably from 1 to 5 years.

In an alternative embodiment, at a pressure in the range 800 to 1200 mbar and a temperature in the range 2° C. to 50° C., the dispersion in accordance with the invention comprises or is a microemulsion, wherein the microemulsion preferably comprises droplets with a droplet size of less than 1 μm, preferably less than 350 nm, preferably less than 100 nm, more preferably in the range 1 nm to 95 nm inclusive, more preferably from 5 nm to 50 nm inclusive.

Preferably, in a microemulsion, the dispersed phase is a liquid phase which is distributed in another liquid phase (dispersion medium), wherein the at least one photosensitizer is preferably dissolved in the dispersed phase, the dispersion medium, or in both phases.

A microemulsion in accordance with the invention preferably further comprises at least one liquid non-polar phase. Preferably, the at least one liquid non-polar phase is in the liquid physical state at a temperature in the range 0° C. to 100° C. and a pressure in the range 800 to 1200 mbar.

Preferably, the at least one liquid non-polar phase comprises at least one non-polar solvent, preferably an aprotic non-polar solvent.

Preferably, a microemulsion in accordance with the invention comprises the at least one non-polar solvent in a proportion of at least 0.1% by weight, preferably at least 0.5% by weight, more preferably at least 1% by weight, more preferably at least 4% by weight, more preferably at least 10% by weight, more preferably at least 35% by weight, more preferably at least 50% by weight, more preferably at least 51% by weight, respectively with respect to the total weight of the microemulsion.

Preferably, a microemulsion in accordance with the invention comprises the at least one non-polar solvent in a proportion in the range 0.1% by weight to 99.8% by weight, preferably in the range 0.5% by weight to 99% by weight, more preferably in the range 1% by weight to 96% by weight, more preferably in the range 1.5% by weight to 90% by weight, more preferably in the range 3% by weight to 80% by weight, more preferably in the range 5% by weight to 75% by weight, more preferably in the range 10% by weight to 60% by weight, more preferably in the range 12% by weight to 49% by weight, respectively with respect to the total weight of the microemulsion.

Preferably, the at least one non-polar solvent is selected from the group which consists of alkanes containing 6 to 30 carbon atoms, monocarboxylic acid esters containing 4 to 20 carbon atoms, polycarboxylic acid esters containing 6 to 20 carbon atoms and mixtures thereof.

Preferably, the aforementioned alkanes, monocarboxylic acid esters and polycarboxylic acid esters have a solubility in the at least one polar solvent, preferably water, at a temperature in the range 2° C. to 50° C. and a pressure in the range 800 to 1200 mbar, of less than 1 g per L of polar solvent, preferably water. More preferably, the aforementioned alkanes, monocarboxylic acid esters and polycarboxylic acid esters are insoluble in the polar solvent, preferably water, at a temperature in the range 10° C. to 25° C. and a pressure in the range 800 to 1200 mbar.

Preferred suitable alkanes, monocarboxylic acid esters and polycarboxylic acid esters have a boiling point (BP) of more than 80° C., preferably of more than 100° C. Preferably, the alkanes, monocarboxylic acid esters and polycarboxylic acid esters have a melting point (MP) below 20° C., preferably below 10° C., more preferably below 0° C.

Preferred suitable alkanes are acyclic alkanes, which may be linear or branched, containing 5 to 30 carbon atoms, preferably containing 6 to 25 carbon atoms, more preferably 8 to 20 carbon atoms, cyclic alkanes containing 5 to 13 carbon atoms, more preferably 6 to 12 carbon atoms, or mixtures thereof.

Suitable alkanes may be unsubstituted, or substituted with fluorine atoms. Suitable preferred fluorine-substituted alkanes are perfluoroalkanes containing 5 to 20 carbon atoms, for example perfluoroheptane, perfluorooctane, perfluorononane, perfluorodecane, perfluorodecalin or mixtures thereof.

Preferred suitable cyclic alkanes are cyclohexane, cycloheptane, cyclooctane, cyclononane, cyclodecane, cycloundecane or mixtures thereof.

Suitable cyclic alkanes may furthermore be substituted with acyclic alkyl residues containing 1 to 6 carbon atoms, for example methyl, ethyl, n-propyl, i-propyl, n-butyl, sec-butyl, tert-butyl, n-pentyl or combinations thereof, and will, for example, be selected from the group which consists of ethylcyclopentane, propylcyclopentane, n-butylcyclopentane, sec-butylcyclopentane, tert-butylcyclopentane, n-pentylcyclopentane, methylcyclohexane, ethylcyclohexane, propylcyclohexane, n-butylcyclohexane, sec-butylcyclohexane, tert-butylcyclohexane, n-pentylcyclohexane, and mixtures thereof.

More preferred suitable acyclic alkanes are mixtures of liquid acyclic alkanes, which have a melting point (MP) of not more than 20° C.

Preferred mixtures of suitable alkanes are paraffin oils, more preferably white oils. Examples of suitable white oils are medical white oils.

Examples of suitable liquid paraffins are entered in the CAS Registry as CAS-8012-95-1 or in the EINECS Registry as EG 232-384-2. Preferably, they have a density of 0.81-0.89 g/cm³. More preferably, the boiling point of suitable liquid paraffins is over 250° C.

Preferred suitable monocarboxylic acid esters are esters of alkanols, preferably containing 1 to 10 carbon atoms, and alkane monocarboxylic acids preferably containing 2 to 16 carbon atoms, wherein the monocarboxylic acid esters preferably contain 4 to 20 carbon atoms.

Preferably, the aforementioned polycarboxylic acid esters containing 6 to 20 carbon atoms contain 2 to 4 carboxy groups, which are preferably completely esterified.

Preferred suitable polycarboxylic acid esters are diesters of alkane dicarboxylic acids containing 4 to 8 carbon atoms, and alkanols containing 1 to 12 carbon atoms. The alkane dicarboxylic acids may preferably be substituted with OH groups.

Examples of suitable polycarboxylic acid esters are dimethyl succinate, diethyl succinate, dimethyl sebacate, diethyl sebacate, diethyl hexyladipate, diisononyl adipate, dimethyl tartrate, diethyl tartrate, diisopropyl tartrate or mixtures thereof.

Unless indicated otherwise, chirality centres can exist in the R or in the S configuration. The invention concerns both the use of optically pure compounds and also the use of mixtures of stereoisomers, such as mixtures of enantionmers and diastereomers, in any ratio.

As an example, diethyl tartrate may exist as the (2S,3S)-tartaric acid diethyl ester, (2R,3R)-tartaric acid diethyl ester, (2R,3S)-tartaric acid diethyl ester, or as a mixture thereof.

Preferably, a microemulsion is an emulsion which is thermodynamically stable at a temperature in the range 2° C. to 50° C. and a pressure in the range 800 to 1200 mbar and in which the dispersed phase forms small domains (“droplets”) which do not scatter incident visible light. Preferably, a microemulsion in accordance with the invention is transparent.

Preferably, a microemulsion in accordance with the invention, which is preferably an oil-in-water (O/W) microemulsion, a water-in-oil (W/O) microemulsion or a bicontinuous microemulsion, preferably an oil-in-water (O/W) microemulsion or a water-in-oil (W/O) microemulsion, comprises:
(a) at least one photosensitizer, which is more preferably selected from the group which consists of the aforementioned phenalenones, the aforementioned curcumins, the aforementioned flavins, the aforementioned porphyrins, the aforementioned porphycenes, the aforementioned xanthene dyes, the aforementioned coumarins, the aforementioned phthalocyanines, the aforementioned phenothiazine compounds, the aforementioned anthracene dyes, the aforementioned pyrenes, the aforementioned fullerenes, the aforementioned perylenes and mixtures thereof, preferably from the aforementioned phenalenones, the aforementioned curcumins, the aforementioned flavins, the aforementioned porphyrins, the aforementioned phthalocyanines, the aforementioned phenothiazine compounds and mixtures thereof, more preferably from the aforementioned phenalenones, the aforementioned curcumins, the aforementioned flavins and mixtures thereof, more preferably from the compounds with formulae (2) to (28), (32) to (49), (51) to (64), (75) to (105) and mixtures thereof,
(b) at least one polar solvent, preferably water,
(c) at least one surfactant, which is selected from the group which consists of the aforementioned non-ionic surfactants, the aforementioned anionic surfactants, the aforementioned cationic surfactants, the aforementioned amphoteric surfactants and mixtures thereof, preferably from the aforementioned non-ionic surfactants, the aforementioned anionic surfactants and mixtures thereof, and
(d) at least one non-polar solvent, which is more preferably selected from the group which consists of the aforementioned acyclic alkanes containing 5 to 30 carbon atoms, the aforementioned cyclic alkanes containing 5 to 13 carbon atoms, the aforementioned perfluoroalkanes containing 5 to 20 carbon atoms, the aforementioned monocarboxylic acid esters containing 4 to 20 carbon atoms, the aforementioned polycarboxylic acid esters containing 6 to 20 carbon atoms, and mixtures thereof.

Preferably, a microemulsion in accordance with the invention further comprises:
(e) at least one alkanol, which is selected from the group which consists of the aforementioned alkanols containing 2 to 12 carbon atoms and preferably containing 1 to 6 OH groups, and mixtures thereof.

Preferably, the at least one surfactant is selected from the group which consists of the aforementioned anionic surfactants and mixtures thereof, and the microemulsion in accordance with the invention further comprises at least one alkanol which is selected from the group which consists of the aforementioned alkanols containing 2 to 12 carbon atoms and preferably containing 1 to 6 OH groups, and mixtures thereof.

Preferably, a microemulsion in accordance with the invention may comprise or consist of an oil-in-water (O/W) microemulsion, a water-in-oil (W/O) microemulsion or a bicontinuous microemulsion, preferably an oil-in-water (O/W) microemulsion or a water-in-oil (W/O) microemulsion.

A bicontinuous microemulsion preferably comprises two domains, a hydrophobic and a hydrophilic domain, in the form of extensive adjacent and intertwined domains, on the interfaces of which stabilizing surface-active surfactants are concentrated in a monomolecular layer.

In an alternative embodiment, the microemulsion in accordance with the invention may comprise or consist of an oil-in-water (O/W) microemulsion, wherein the dispersed phase comprises at least one liquid non-polar phase which more preferably comprises at least one non-polar solvent which is selected from the group which consists of the aforementioned alkanes containing 6 to 30 carbon atoms, the aforementioned monocarboxylic acid esters containing 4 to 20 carbon atoms, the aforementioned polycarboxylic acid esters containing 6 to 20 carbon atoms, and mixtures thereof. Preferably, the dispersion medium for the oil-in-water (O/W) microemulsion comprises at least one polar solvent, preferably water.

Preferably, an oil-in-water (O/W) microemulsion in accordance with the invention comprises the at least one non-polar solvent in a proportion in the range 0.1% by weight to 49.9% by weight, preferably in the range 0.5% by weight to 48% by weight, more preferably in the range 1% by weight to 45% by weight, more preferably in the range 3% by weight to 40% by weight, more preferably in the range 5% by weight to 35% by weight, more preferably in the range 7% by weight to 30% by weight, respectively with respect to the total weight of the microemulsion.

Preferably, an oil-in-water (O/W) microemulsion in accordance with the invention further comprises the at least one polar solvent, preferably water, in a proportion in the range 50% by weight to 99.8% by weight, preferably in the range 51% by weight to 99% by weight, more preferably in the range 52% by weight to 96% by weight, more preferably in the range 53% by weight to 90% by weight, more preferably in the range 54% by weight to 85% by weight, respectively with respect to the total weight of the microemulsion.

Preferably, an oil-in-water (O/W) microemulsion in accordance with the invention further comprises the at least one surfactant in a proportion in the range 0.1% by weight to 45% by weight, preferably in the range 0.5% by weight to 40% by weight, more preferably in the range 1% by weight to 35% by weight, more preferably in the range 3% by weight to 30% by weight, more preferably in the range 5% by weight to 27% by weight, more preferably in the range 7% by weight to 25% by weight, more preferably in the range 10% by weight to 20% by weight, respectively with respect to the total weight of the microemulsion.

Preferably, an oil-in-water (O/W) microemulsion in accordance with the invention further comprises the at least one alkanol in a proportion in the range 0% by weight to 50% by weight, preferably in the range 0.1% by weight to 40% by weight, more preferably in the range 0.5% by weight to 35% by weight, more preferably in the range 1% by weight to 30% by weight, more preferably in the range 1.5% by weight to 25% by weight, more preferably in the range 5% by weight to 20% by weight, more preferably in the range 7% by weight to 19% by weight, more preferably in the range 10% by weight to 17% by weight, respectively with respect to the total weight of the microemulsion.

In a further alternative embodiment, the microemulsion in accordance with the invention comprises or consists of a water-in-oil (W/O) microemulsion, wherein the dispersed phase comprises at least one polar solvent, preferably water. Preferably, the dispersion medium for the water-in-oil (W/O) microemulsion comprises at least one liquid non-polar phase, which more preferably comprises at least one non-polar solvent which is selected from the group which consists of the aforementioned acyclic alkanes containing 5 to 30 carbon atoms, the aforementioned cyclic alkanes containing 5 to 13 carbon atoms, the aforementioned perfluoroalkanes containing 5 to 20 carbon atoms, the aforementioned monocarboxylic acid esters containing 4 to 20 carbon atoms, the aforementioned polycarboxylic acid esters containing 6 to 20 carbon atoms, and mixtures thereof.

Preferably, a water-in-oil (W/O) microemulsion in accordance with the invention comprises the at least one polar solvent, preferably water, in a proportion in the range 0.1% by weight to 49.9% by weight, preferably in the range 0.5% by weight to 48% by weight, more preferably in the range 1% by weight to 45% by weight, more preferably in the range 3% by weight to 40% by weight, more preferably in the range 5% by weight to 35% by weight, more preferably in the range 7% by weight to 30% by weight, respectively with respect to the total weight of the microemulsion.

Preferably, a water-in-oil (W/O) microemulsion in accordance with the invention further comprises the at least one non-polar solvent in a proportion in the range 50% by weight to 99.8% by weight, preferably in the range 51% by weight to 99% by weight, more preferably in the range 52% by weight to 96% by weight, more preferably in the range 55% by weight to 90% by weight, more preferably in the range 60% by weight to 80% by weight, respectively with respect to the total weight of the microemulsion.

Preferably, a water-in-oil (W/O) microemulsion in accordance with the invention further comprises the at least one surfactant and the at least one alkanol in the aforementioned proportions by weight, respectively with respect to the total weight of the microemulsion.

Preferably, a water-in-oil (W/O) microemulsion in accordance with the invention or an oil-in-water (O/W) microemulsion in accordance with the invention further comprises at least one metallic salt which is soluble in the at least one polar solvent, preferably water, the metal being selected from the group which consists of metals from main groups 1 to 3 of the periodic table of the elements, preferably alkali metals or alkaline-earth metals, and at least one anion which is selected from the group which consists of fluoride, chloride, bromide, iodide, sulphate, hydrogen sulphate, phosphate, dihydrogen phosphate, hydrogen phosphate, tosylate, mesylate, formate, acetate, propionate, butanoate, oxalate, tartrate, fumarate, benzoate, citrate and/or mixtures thereof, more preferably chloride, sulphate, hydrogen sulphate, formate, acetate, benzoate, citrate and/or mixtures thereof.

Preferably, a water-in-oil (W/O) microemulsion in accordance with the invention or an oil-in-water (O/W) microemulsion in accordance with the invention comprises the at least one soluble salt in a proportion in the range 0% by weight to 20% by weight, preferably in the range 0.5% by weight to 15% by weight, more preferably in the range 0.7% by weight to 10% by weight, more preferably in the range 1% by weight to 7% by weight, more preferably in the range 1.5% by weight to 5% by weight, respectively with respect to the total weight of the microemulsion.

Preferably, the microemulsion in accordance with the invention is a thermodynamically stable monophase, more preferably at a temperature in the range 2° C. to 50° C. and a pressure in the range 800 to 1200 mbar.

More preferably, the microemulsion contains droplets with a droplet size of less than 350 nm, preferably less than 100 nm, more preferably in the range 1 nm to 95 nm inclusive, more preferably from 5 nm to 50 nm inclusive.

The inventors have surprisingly established that providing at least one photosensitizer in a microemulsion, wherein the at least one photosensitizer is preferably dissolved in the microemulsion, improves the application characteristics of the photosensitizer.

As an example, the at least one photosensitizer may be provided in a concentrate which contains a higher concentration of the photosensitizer than is required in a solution that is ready for use, for example.

Preferably, a concentrate will also be in the form of a microemulsion. The inventors have surprisingly established that a microemulsion in accordance with the invention can be diluted with many times the quantity of water, preferably 4 to 16 times the quantity of water, respectively with respect to the volume of the concentrate to be diluted, without the wettability of the dilution obtained being significantly deteriorated compared with the wettability of the concentrate.

In an alternative embodiment, the dispersion in accordance with the invention comprises or is a gel, preferably a lyogel, at a pressure in the range 800 to 1200 mbar and a temperature in the range 2° C. to 50° C.

Preferably in a gel, preferably a lyogel, the dispersed phase comprises a solid component which is distributed in a liquid phase (dispersion medium). Preferably, the at least one photosensitizer is dissolved in the liquid phase.

Preferably, the solid component thus forms a sponge-like, three-dimensional network with pores which are filled with a liquid (lyogel). The liquid component is thus preferably immobilized in the solid component. Both components intertwine with each other, preferably completely (bicoherence).

Preferably, a gel in accordance with the invention, preferably a lyogel, comprises:
(a) at least one photosensitizer, which is more preferably selected from the group which consists of the aforementioned phenalenones, the aforementioned curcumins, the aforementioned flavins, the aforementioned porphyrins, the aforementioned porphycenes, the aforementioned xanthene dyes, the aforementioned coumarins, the aforementioned phthalocyanines, the aforementioned phenothiazine compounds, the aforementioned anthracene dyes, the aforementioned pyrenes, the aforementioned fullerenes, the aforementioned perylenes and mixtures thereof, preferably from the aforementioned phenalenones, the aforementioned curcumins, the aforementioned flavins, the aforementioned porphyrins, the aforementioned phthalocyanines, the aforementioned phenothiazine compounds and mixtures thereof, more preferably from the aforementioned phenalenones, the aforementioned curcumins, the aforementioned flavins and mixtures thereof, more preferably from the compounds with formulae (2) to (25), (32) to (49), (51) to (64), (75) to (105), and mixtures thereof,
(b) at least one polar solvent, preferably water,
(c) at least one surfactant which is selected from the group which consists of the aforementioned non-ionic surfactants, the aforementioned anionic surfactants, the aforementioned cationic surfactants, the aforementioned amphoteric surfactants and mixtures thereof, preferably from the aforementioned non-ionic surfactants, the aforementioned anionic surfactants and mixtures thereof, and
(d) at least one gelling agent.

Suitable gelling agents are preferably selected from the group which consists of polyacrylic acids, polyacrylamides, alginates, cellulose ethers, and mixtures.

Examples of suitable cellulose ethers are carboxymethyl cellulose (CMC), methyl cellulose (MC), ethyl cellulose (EC), hydroxyethyl cellulose (HEC), hydroxyethylmethyl cellulose (NEMC) or hydroxypropylmethyl cellulose (HPMC), hydroxyethylmethyl celluloses, hydroxypropylmethyl celluloses, ethylhydroxyethyl celluloses, carboxymethylhydroxyethyl celluloses, or mixtures thereof.

Examples of suitable carboxyyinylpolymers are polyacrylic acids, acrylate copolymers or mixtures thereof.

Preferably, a gel in accordance with the invention, preferably a lyogel, comprises the at least one gelling agent in a proportion in the range 0.1% by weight to 49.9% by weight, preferably in the range 0.5% by weight to 45% by weight, more preferably in the range 1% by weight to 41% by weight, more preferably in the range 2% by weight to 37% by weight, more preferably in the range 3% by weight to 25% by weight, more preferably in the range 5% by weight to 15% by weight, respectively with respect to the total weight of the gel, preferably the lyogel.

Preferably, a gel in accordance with the invention, preferably a lyogel, further comprises: (e) at least one pH-regulating substance, which is preferably an inorganic acid, an organic acid, an inorganic base, an organic base, or a mixture thereof.

Preferably, the pH of the gel, preferably the lyogel, is in the range 4 to 11, preferably 6 to 10, at a temperature in the range 2° C. to 50° C. and a pressure in the range 800 to 1200 mbar.

Examples of suitable inorganic acids are phosphoric acid, sulphuric acid, hydrochloric acid or mixtures thereof.

Examples of suitable organic acids are acetic acid, sulphuric acid, toluenesulphonic acid, citric acid, barbituric acid, 4-(2-hydroxyethyl)-1-piperazineethanesulphonic acid, 4-(2-hydroxyethyl)-piperazin-1-propanesulphonic acid, 2-(N-morpholino)ethanesulphonic acid, or mixtures thereof.

Examples of suitable inorganic bases are phosphates, hydrogen phosphates, dihydrogen phosphates, sulphates, hydrogen sulphates, ammonia, NaOH, KOH, or mixtures thereof.

An example of a suitable organic base is tris(hydroxymethyl)aminomethane, N-methylmorpholine, triethylamine, pyridine or mixtures thereof.

Preferably, a gel in accordance with the invention, preferably a lyogel, further comprises: (f) at least one soluble metallic salt which is soluble in a polar solvent, preferably water, the metal being selected from the group which consists of metals from main groups 1 to 3 of the periodic table of the elements, preferably alkali metals or alkaline-earth metals, and at least one anion which is selected from the group which consists of fluoride, chloride, bromide, iodide, sulphate, hydrogen sulphate, phosphate, dihydrogen phosphate, hydrogen phosphate, tosylate, mesylate, formate, acetate, propionate, butanoate, oxalate, tartrate, fumarate, benzoate, citrate and/or mixtures thereof, more preferably chloride, sulphate, hydrogen sulphate, formate, acetate, benzoate, citrate and/or mixtures thereof.

Preferably, a gel in accordance with the invention, preferably a lyogel, comprises the at least one soluble salt in a proportion in the range 0% by weight to 20% by weight, preferably in the range 0.5% by weight to 15% by weight, more preferably in the range 0.7% by weight to 10% by weight, more preferably in the range 1% by weight to 7% by weight, more preferably in the range 1.5% by weight to 5% by weight, respectively with respect to the total weight of the inventive gel, preferably a lyogel.

Preferably, the gel, preferably the lyogel, has a dynamic viscosity in the range 1000 Pas to 5000 Pas.

The active or passive ingress, adhesion and proliferation of pathogens in a host is termed an infection. Sources of infectious particles are ubiquitous. Thus, for example, the human body is colonized by a large number of microorganisms which are usually kept under control by the normal metabolism and an intact immune system. However, when the immune system is weakened, for example, substantial proliferation of the pathogens may occur and, depending on the type of the pathogen, various symptoms of disease may manifest themselves. The medical profession has specific remedies prepared for many diseases caused by pathogens, for example antibiotics against bacteria, or antimycotics against fungi or antivirals against viruses. However, when these remedies are employed, an increase in the occurrence of resistant pathogens is observed which sometimes also have resistance to more than one remedy. Because of the occurrence of these resistant or multi-resistant pathogens, the therapy of infectious diseases is becoming more and more difficult. The clinical consequence of resistance is indicated by a failure of treatment, especially in immunosuppressed patients.

Single-celled or multi-celled microorganisms can trigger infectious diseases. By application of at least one pathogen-specific remedy, for example an antibiotic, antimycotic or antiviral, the number of pathogens can be reduced and/or the pathogen can be inactivated. The application of a pathogen-specific remedy may be systemic and/or topical.

In systemic application, the pathogen-specific remedy is transferred into the blood and/or lymph system of the body to be treated and thus distributed through the entire body. In the systemic administration of the pathogen-specific remedy, degradation of the remedy and/or side effects, for example by a biochemical transformation (metabolization) of the remedy, may occur.

In the topical application of the pathogen-specific remedy, the remedy is applied where it is to act therapeutically, for example onto an infected part of the skin, while healthy skin is not affected. In this manner, systemic side effects can be largely avoided.

Superficial skin or soft tissue infections do not necessarily have to be treated with a systemic application of a pathogen-specific remedy, because the remedy can be applied directly to the infected parts of the skin.

Known pathogen-specific remedies exhibit side effects and interactions, some of which may be severe, both with systemic and with topical application. Furthermore, with topical application, an inadmissible intake of medication (compliance) of the patient, in particular when using antibiotics, may give rise to resistance.

An alternative here is the photodynamic inactivation of microorganisms, because resistance to photodynamic inactivation is unknown. Independently of the type of the microorganisms to be combatted and the associated infectious diseases, the number of pathogens is reduced and/or the pathogens are eradicated. As an example, mixtures of various microorganisms, for example fungi and bacteria or different bacterial strains, can be controlled.

The objective of the present invention is also accomplished by the provision of a dispersion as claimed in one of claims 1 to 14, for use in photodynamic therapy for the inactivation of microorganisms, which preferably are selected from the group consisting of viruses, archaea, bacteria, bacterial spores, fungi, fungal spores, protozoa, algae and blood-borne parasites, wherein the dispersion is preferably used in the treatment and/or prophylaxis of a disease of dental tissue and/or of the periodontium.

The objective of the present invention is also accomplished by the provision of a method for the photodynamic inactivation of microorganisms, which preferably include viruses, archaea, bacteria, bacterial spores, fungi, fungal spores, protozoa, algae, blood-borne parasites or combinations thereof, wherein the method comprises the following steps:

(A) bringing the microorganisms into contact with at least one dispersion as claimed in one of claims 1 to 14, and

(B) irradiating the microorganisms and the at least one photosensitizer contained in the dispersion with electromagnetic radiation of a suitable wavelength and energy density.

Preferably, the method in accordance with the invention is carried out in order to inactivate microorganisms during photodynamic therapy of a patient and/or photodynamic decontamination of at least one surface of an article and/or at least one surface of an area.

In a preferred embodiment of the method in accordance with the invention, irradiation of the microorganisms and of the at least one photosensitizer with electromagnetic radiation of a suitable wavelength and energy density is carried out in the presence of at least one oxygen-donating compound, preferably peroxide, and/or at least one oxygen-containing gas, preferably oxygen.

The at least one oxygen-donating compound and/or the at least one oxygen-containing gas may preferably be applied before or during step (B) of the method in accordance with the invention.

By adding extra oxygen in the form of at least one oxygen-containing compound and/or at least one oxygen-containing gas before or during irradiation of the microorganisms and of the at least one photosensitizer with electromagnetic radiation of a suitable wavelength and energy density, the yield of reactive oxygen species (ROS) formed, preferably oxygen radicals and/or singlet oxygen, is increased.

The objective of the present invention is also accomplished by the use of at least one dispersion as claimed in one of claims 1 to 14 for the inactivation of microorganisms, which preferably comprise viruses, archaeae, bacteria, bacterial spores, fungi, fungal spores, protozoa, algae, blood-borne parasites or combinations thereof.

A dispersion for use in accordance with the invention has a high yield of singlet oxygen following irradiation with electromagnetic radiation of a suitable wavelength.

In the method in accordance with the invention and/or the use in accordance with the invention, the electromagnetic irradiation is preferably in the visible, ultraviolet and/or infrared spectral range. More preferably, the electromagnetic irradiation has a wavelength in the range from 280 to 1000 nm, more preferably from 380 to 1000 nm.

More preferably, the electromagnetic irradiation has an energy density in the range from 1 μW/cm² to 1 kW/cm², more preferably from 1 mW/cm² to 100 W/cm², more preferably from 2 mW/cm² to 50 W/cm², more preferably from 6 mW/cm² to 30 W/cm², more preferably from 7 mW/cm² to 25 W/cm².

The irradiation period may be varied as a function of the type of microorganisms and/or the severity of the infection. Preferably, the irradiation period is in the range from 1 μs to 1 h, more preferably from 1 ms to 1000 s.

As an example, the irradiation procedure carried out for the irradiation may be that described in either WO 96/29943 A1, EP 0 437 183 B1 or WO 2013/172977 A1.

Preferably, the irradiation device also comprises a device for releasing the at least one oxygen-containing compound, preferably peroxide, and/or the at least one oxygen-containing gas, preferably oxygen.

Preferably, the electromagnetic radiation is produced by a source of radiation which is selected from the group consisting of artificial sources of irradiation, for example UV lamps, IR lamps, fluorescent lamps, light-emitting diodes, lasers or chemical light.

Furthermore, the inventors have surprisingly discovered that at least one photosensitizer contained in the dispersion in accordance with the invention exhibits a high affinity for microorganisms.

Because of the affinity, the at least one photosensitizer contained in the dispersion in accordance with the invention can effectively bind to microorganisms and locally produce sufficient singlet oxygen to inactivate the microorganisms, preferably to eradicate them.

Furthermore, because the at least one photosensitizer is provided in the form of the dispersion in accordance with the invention, the half-life of the locally formed singlet oxygen is significantly extended following irradiation with electromagnetic radiation of a suitable wavelength and energy density.

Following irradiation of the dispersion in accordance with the invention with electromagnetic radiation of a suitable wavelength and energy density, the microorganisms are inactivated, preferably eradicated, by the reactive oxygen species (ROS), preferably oxygen radicals and/or singlet oxygen, which are produced.

Preferably, the extension of the half-life of the locally formed singlet oxygen following irradiation with electromagnetic radiation of a suitable wavelength and energy density means that the progress of the inactivation of microorganisms or their decolonization can be accelerated.

In the context of the invention, the term “decolonization” should be understood to mean the removal, preferably complete removal, of microorganisms.

Preferably, body surfaces, for example skin or mucous membranes, of humans and animals, preferably mammals, can be treated. In this preferred embodiment, at least one dispersion for use in accordance with the invention is used for the decontamination and/or decolonization of skin or soft tissue surfaces, wherein preferably, the integrity of the skin is maintained.

In a further preferred embodiment, a dispersion for use in accordance with the invention is used for local and/or topical, preferably nasal, oral, anal, vaginal or dermal application.

The term “topical application” should also be understood to mean application on or in the ear, preferably the outer ear. The outer ear comprises the ear cartilage, the auricle, the earlobe, the outer auditory or ear canal and the outside of the eardrum.

The term “topical application” should also be understood to mean application on or in the nose and/or the paranasal sinuses such as, for example, the maxillary sinus, the frontal sinus and/or the sphenoid sinus.

The term “topical application” should also be understood to mean application to the surface of the eye, preferably the outer, apical side of the epithelial layer of the cornea and/or the outer surface of the associated organs of the eye, preferably the tear ducts, the conjunctiva and/or the eyelids.

The term “topical application” should also be understood to mean application to the outer, apical side of the epithelia of hollow organs, for example the oesophagus, the gastro-intestinal tract, the gall bladder, the bile ducts, the larynx, the airways, the bronchia, the ovaries, the uterus, the vagina, the ureter, the bladder or the urethra.

The term “topical application” should also be understood to mean application to or into teeth, for example in a root canal and/or a root cavity and/or tooth fissure, or gingival pockets and/or bone fenestrations.

In a further preferred embodiment, a dispersion for use in accordance with the invention is used for the production of a pharmaceutical preparation for the prophylaxis and/or treatment of an infectious, preferably viral, bacterial and/or mycotic skin disease which is preferably selected from the group which consists of staphylococcal scalded skin syndrome, impetigo, skin abscesses, boils, carbuncles, phlegmon, cellulitis, acute lymphadenitis, pilonidial disease, pyoderma, dermatitis purulenta, dermatitis septica, dermatitis suppurativa, erythrasma, erysipelas, acne vulgaris or fungal infections.

In a further preferred embodiment, a dispersion for use in accordance with the invention is used for the production of a pharmaceutical preparation for healing wounds, for example in the event of healing disorders following surgical intervention.

Preferably, at least one dispersion for use in accordance with the invention is used for the decontamination and/or reduction of the bacterial count in infected wounds.

In a further preferred embodiment, at least one dispersion for use in accordance with the invention is used for the production of a pharmaceutical preparation for the prophylaxis and/or treatment of infectious diseases, preferably viral, bacterial and/or mycotic, of the ear, the upper airways, the oral cavity, the throat, the larynx, the lower airways and/or the oesophagus.

The predominance of pathogenic microorganisms is, for example, the main cause of infection in the oral cavity. In this regard, the problem arises that the microorganisms are organized synergistically into extremely complex biofilms. These biofilms, for example plaque or tartar, consist of a plurality of complex layers and the proteins, carbohydrates, phosphates and microorganisms contained therein. Tartar occurs in particular when the surface of the tooth cannot be kept free of deposits by natural or artificial cleaning. This situation makes it difficult to obtain access to the microorganisms which are bound into the biofilm.

Conventional therapies such as antibiotics and mouthwashes or mechanical tooth cleaning can only be used to a limited extent, because either they cannot affect the bacteria directly, for example during tooth cleaning, are difficult to dose and apply, for example with antibiotics and mouthwashes, or a general application is not justified because of negative side effects.

As an example, in the United States, 20 million root canal treatments are carried out annually, within which more than 2 million endodontic re-treatments are carried out which could be avoided by improved decontamination of the root canals.

Preferably, the method in accordance with the invention and the use in accordance with the invention is suitable for the effective elimination of microorganisms in the root canal systems of a human tooth, encompassing the root canal and dental canaliculi.

In a further preferred embodiment, at least one dispersion for use in accordance with the invention is used for the production of a pharmaceutical preparation for the treatment and/or prophylaxis of an infectious disorder, preferably viral, bacterial and/or mycotic, of the tooth tissue, preferably plaque, caries or pulpitis, and/or infectious disorder, preferably viral, bacterial and/or mycotic, of the periodontal apparatus, preferably gingivitis, paradontitis, endodontitis or periimplantitis.

In a further preferred embodiment, at least one dispersion for use in accordance with the invention is used in cleaning teeth, dental prostheses and/or braces, or for the nasal decolonization of microorganisms.

As an example, methicillin-resistant staphylococcus aureus (MRSA) strains persist for a month during the course of nasal colonization and also have a high resistance to the environment. Thus, a nasal decolonization, i.e. removal of microorganisms, also reduces the colonization in other sites on the body.

In a further preferred embodiment, at least one dispersion for use in accordance with the invention is used in the inactivation of microorganisms in a biological fluid, preferably medical blood products.

Suitable equipment for irradiating a biological fluid is known to the person skilled in the art and has been described, for example, in WO 99/43790 A1, US 2009/0010806 A1 or WO 2010/141564 A2.

Examples of suitable biological fluids are blood and blood products, including frozen fresh plasma, erythrocyte concentrate, thrombocyte concentrate, granulocyte concentrate, thrombocyte-rich plasma, stem cell preparations, concentrates of individual coagulation factors, human albumin, immunoglobulins, fibrin adhesive, antithrombin, protein C, protein S, fibrinolytics or combinations thereof.

In a preferred embodiment, at least one dispersion for use in accordance with the invention is used for the photodynamic decontamination of surfaces of all types. Photodynamic decontamination of surfaces causes photodynamic inactivation of microorganisms on the treated surface.

Examples of suitable surfaces are surfaces formed from plastic, metal, glass, textiles, wood, stone or combinations thereof.

More preferably, at least one dispersion in accordance with the invention is used in the photodynamic decontamination, surface cleaning and/or coating, preferably of medical products, electronic devices, hygiene articles, food packaging, foodstuffs, furniture, building materials or areas, for example floors, walls and/or windows.

More preferably, articles are treated which have a thermally limited shelf life, for example articles formed from thermoplastic plastics or which are attacked by disinfectants.

Articles which have a thermally limited shelf life cannot be sufficiently sterilized, for example, because they lose their shape or become brittle at higher temperatures.

Furthermore, the improper and/or excessive use of disinfectants can lead to the build-up of resistance by selection of more robust microorganisms if, for example, the concentration of the substance and exposure time and thus the pathogen-reducing action is too small.

In a further preferred embodiment, the method in accordance with the invention is used to prevent a bacterial infection, for example prior to implantation or after successful decolonization, for example to prevent a fresh colonization with disease-inducing microorganisms such as, for example, pathogenic paradontal microorganisms.

In order to avoid infections by microorganisms, the method in accordance with the invention may also be used for the decolonization of surfaces.

As an example, contact by immunosuppressed patients with contaminated articles often leads to the build-up of an infection, because immunosuppressed patients are usually susceptible to infections, for example even from low bacterial counts. In particular, the surfaces of medical products, preferably medical accessories or dental accessories, more preferably invasive medical accessories such as catheters, hollow probes, tubes or needles, have to be disinfected before they are introduced into the human body.

Thus, in a further preferred embodiment, at least one dispersion for use in accordance with the invention is used for the inactivation of microorganisms on surfaces of medical products, preferably invasive medical accessories such as, for example, contact lenses, surgical instruments, dental drills, dental mirrors, curettes, dental files, catheters, hollow probes, tubes or needles.

Preferably, the medical products are selected from wound dressings, bandages, surgical instruments, catheters, hollow probes, tubes or needles.

More preferably, the term “medical products” should also be understood to include dental bridges, impression trays, braces, occlusal splints or dentures, for example prostheses, crowns or implants, as well as hearing aids or contact lenses, for example.

Preferably, by means of a treatment of the surface of articles of all types with at least one dispersion in accordance with the invention on the surface of medical products and subsequent irradiation with electromagnetic radiation of a suitable wavelength and energy density, colonization of microorganisms on the treated surfaces is reduced, preferably prevented.

Preferably, the surface treatment is carried out by atomization, painting, injection, spraying, immersion or combinations thereof.

The irradiation may be carried out directly following treatment of the surface with at least one dispersion for use in accordance with the invention and/or at a later point in time, before or during the use of the treated article, for example a medical product.

In a further preferred embodiment, at least one dispersion in accordance with the invention is used for the inactivation of microorganisms on surfaces of food packaging,

Examples of suitable food packaging include containers produced from glass, metal, plastic, paper, card or combinations thereof.

Before filling with a foodstuff or beverage, suitable containers may, for example, be treated with at least one dispersion for use in accordance with the invention and subsequently irradiated with a suitable source of radiation which produces electromagnetic radiation of a suitable wavelength and energy density. Subsequently, the appropriate foodstuff or beverage can be placed in the decontaminated container and the container can be sealed.

In a further preferred embodiment, at least one dispersion in accordance with the invention is used for the inactivation of microorganisms on surfaces of foodstuffs.

Examples of suitable foodstuffs are foodstuffs such as meat, fish, eggs, seeds, grain, nuts, berries, spices, fruit or vegetables which may come into contact with pathogenic bacterial species such as Salmonella, Clostridium, Escherichia coli or Camphylobacter species. Advantageously, hatching eggs may also be photodynamically decontaminated.

The term “gastro-intestinal infection” is used to describe a group of diseases which are primarily distinguished by symptoms in the upper gastro-intestinal tract such as vomiting, diarrohea and stomach pain. Gastro-intestinal infections are caused by viruses, bacteria or parasites. The pathogens are usually picked up via contaminated water and/or contaminated food.

The best known sources of gastro-intestinal infections include, for example, Salmonella, Campylobacter species or Escherichia coli species such as, for example, enterohaemorrhagic Escherichia coli (EHEC). Diarrhoea and vomiting due to food poisoning are primarily caused by staphylococci.

Most usually, pathogens of gastro-intestinal infections such as Salmonella, for example, get into the digestive tract of human beings via foodstuffs. The inventors have discovered that using the method in accordance with the invention can efficiently remove microorganisms from the surface of foodstuffs.

Salmonella, for example, are bacteria which occur worldwide. A Salmonella disease is a typical infection of foodstuffs which causes diarrohea. The pathogens multiply in the gastro-intestinal tract of humans and animals. Salmonella can multiply rapidly on non-chilled foodstuffs. Under certain circumstances, the bacteria get into food due to poor kitchen hygiene, for example via dirty cutting boards and/or knives.

Examples of foodstuffs which are often loaded with Salmonella are raw, i.e. incompletely cooked eggs and egg products such as mayonnaise, creams or salads based on eggs or raw dough. Further examples of foodstuffs which are often loaded with Salmonella are ice cream, raw meat, for example raw mince or tartare, raw sausages, for example smoked sausage or salami. Vegetable foodstuffs may also be colonized with Salmonella.

Campylobacter are globally occurring bacteria which trigger infectious diarrohea. Campylobacter species live mainly in the digestive tract of animals which usually do not become ill themselves. Campylobacter are the most common bacterial cause of diarrohea in Germany.

The main source of infection for Campylobacter is the consumption of foodstuffs which are contaminated with the bacteria. It is often transmitted via poultry meat. Campylobacter cannot multiply in foodstuffs, but Campylobacter can survive for some time in the environment. Again, poor kitchen hygiene can lead to an infection, for example via cutting boards and/or knives which are not adequately cleaned after preparing raw meat.

Examples of foodstuffs which are often contaminated with Campylobacter are insufficiently cooked poultry meat and poultry products, unpasteurized milk or unpasteurized milk products, minced meat which has not been thoroughly cooked or fresh raw sausages such as smoked sausage, and contaminated drinking water, for example from a well system.

Enterohaemorrhagic Escherichia coli (EHEC) is in the gut of ruminants such as cattle, sheep, goats or deer. The bacteria are expelled with the faeces of infected animals. Because EHEC are relatively insensitive, they can survive in the environment for weeks. They are still highly infectious and even a small number of pathogens is sufficient for transmission. The coats of cattle and other ruminants can be contaminated with traces of faeces. By touching and stroking the animals, the bacteria can reach the hands and from there get into the mouth. Even playing in meadows where ruminants have been kept runs the risk of infection for children.

By using the method in accordance with the invention, surfaces of shoes, for example soles, can easily be decontaminated photodynamically.

Furthermore, the inventors have discovered that the method in accordance with the invention is also suitable for the photodynamic decontamination of the surfaces of animal products such as coats, leather, hair, fibres or wool.

As an example, because of poor hand hygiene, the EHEC bacteria may remain on articles which are touched and be spread further from there.

Transfer to human beings can also occur by means of foodstuffs which are eaten raw or have been heated insufficiently. Examples of foodstuffs which are often contaminated with EHEC are unpasteurized milk and unpasteurized milk products, raw or insufficiently cooked meat products such as, for example, ground beef (for example hamburgers) and spreadable raw sausages, for example Teewurst. Vegetable foodstuffs are also often contaminated with EHEC, for example vegetables which are contaminated with the pathogens by fertilization or contaminated water, unpasteurized fruit juices which are produced from contaminated fruit, seeds which are used to cultivate shoots, and all foods onto which the pathogens from contaminated foodstuffs can be transferred directly or indirectly by dirty hands or cooking utensils.

Clostridium difficile is for example, a bacterium which occurs globally. In healthy people, Clostridium difficile is a harmless gut bacterium. If competing types of normal gut flora are suppressed by antibiotics, Clostridium difficile can multiply and produce toxins which under some circumstances can lead to life-threatening diarrohea, for example antibiotic-associated colitis, in particular if an antibiotic-associated diarrohea has already occurred. Clostridium difficile is one of the most common hospital pathogens (nosocomial pathogen). Furthermore, Clostridium difficile can form resistant permanent forms, what are known as spores, by means of which, under certain circumstances, the bacteria can survive for years outside the gastro-intestinal tract. Thus, it is also possible to transmit it via articles and surfaces such as, for example, toilets, door handles, handles and/or hand rails to which the pathogens adhere.

The problems described above can be avoided by using the method in accordance with the invention, because disease-causing pathogens on contaminated surfaces are effectively removed after using the method in accordance with the invention.

In a further preferred embodiment, at least one dispersion in accordance with the invention is used for the inactivation of microorganisms in an area, for example a clean room or an operating theatre. After introduction into the area, for example by misting, spraying, injection or evaporation, the area can be irradiated with a suitable source of radiation which produces electromagnetic radiation of a suitable wavelength and energy density, whereupon the microorganisms present are inactivated.

In a further preferred embodiment, at least one dispersion in accordance with the invention is used for the inactivation of microorganisms in a liquid or liquid preparation. Examples of suitable liquids or liquid preparations are emulsion paints, coolants, cooling lubricants, lubricants, brake liquids, paints, adhesives or oils. Preferably, the liquid preparation is an aqueous preparation.

Preferably, the liquid is water.

In this regard, at least one dispersion in accordance with the invention can be used for the preparation of water for the beverage and food industries, the pharmaceuticals, chemicals and cosmetics industries, and the electronics industry. Furthermore, at least one dispersion for use in accordance with the invention can be used for drinking water and rain water preparation, for the treatment of waste water or for the preparation of water for use in air conditioning technology.

Examples of suitable articles are medical products, foodstuff packaging, hygiene articles, textiles, handles, hand rails, contact lenses, building materials, banknotes, coins, gaming chips, cards, sports equipment, textiles, crockery, cutlery or electronic devices. Other suitable articles are devices or units with water-carrying lines and/or water-carrying containers in which condensed water is formed, for example during operation of the device or the unit.

Examples of suitable articles are seals, membranes, screens, filters, containers and/or pipes for hot water production units, hot water distribution units, heat exchangers, air conditioning units, air humidifiers, chillers, refrigerators, drinks dispensers, washing machines or dryers.

As an example, despite filtration of the air fed in from outside, small quantities of microorganisms can gain ingress into an air conditioning unit and exist there for at least a short period. The metabolic products from these microorganisms could give rise to stale and musty odours.

Furthermore, in order to operate an air conditioning unit, moisture has to be removed from the air and trapped. A large proportion of the condensed water is removed and, for example, runs through a condensed water line. However, residual dampness remains on the surface of the evaporator of the air conditioning unit, in particular when the air conditioning unit is only switched off in a passenger vehicle when the engine is switched off and the temperature can no longer be equilibrated.

The microorganisms which reach the evaporator from the air, for example fungal spores and/or bacteria, now find themselves in an ideal warm, moist climate and can proliferate unchecked.

Since moulds, for example, constitute a risk to health, the air conditioning unit should be decontaminated regularly and any microorganisms present should be eradicated by carrying out the method in accordance with the invention.

When changing the filter of the air conditioning unit, for example the dust and/or pollen filter, again, the filter housing and the surrounding air ducts of the air conditioning unit can be cleaned by using the method in accordance with the invention. By cleaning the evaporator of the air conditioning unit using the method in accordance with the invention, odours which arise in the air conditioning unit can also be removed.

Legionella bacteria are, for example, bacteria which cause different symptoms in human beings, for example flu-like symptoms or severe lung infections. Legionella bacteria preferably multiply at temperatures between 25° C. and 45° C. Particularly in artificial water systems such as water pipes in buildings, the pathogens find good conditions for growth because of the prevailing temperatures. Legionella bacteria can also multiply well in sediments and/or linings in a piping system. Thus, the method in accordance with the invention, for example in combination with a method for removing sediments and/or linings, could be used.

Legionella bacteria are transmitted by atomized, cloudy water. The droplets containing the pathogens can be distributed in the air and breathed in. Examples of possible sources of infection are hot water supplies, in particular showers, air humidifiers or water taps, as well as cooling towers or air conditioning units or other units which atomize water into water droplets, for example misters, mist fountains, water features or the like. Transfer is also possible in swimming baths via waterfalls, slides, whirlpools and/or fountains. Infection with Legionella bacteria is prevented by using the method in accordance with the invention on surfaces of contaminated articles.

The method in accordance with the invention may, for example, be used in equipment or units with water-supplying lines and/or water-supplying containers, for example equipment or units which are used in fish farming.

Epidemic-like diseases of fish are an example of a huge economic threat for all intensively operated fish farms where farmed fish are kept in confined spaces. In order to combat the fish diseases, antibiotics and/or chemical additives are added, for example. Examples of chemical additives which are used are calcium hydroxide, hydrogen peroxide, peracetic acid preparations, copper sulphate, chloramines, sodium carbonate, sodium chloride or formaldehyde.

In order to reduce the use of antibiotics and/or the chemical additives mentioned above, at least one dispersion in accordance with the invention may be used for the photodynamic decontamination of equipment or units in fish farming, for example fish ponds, pools, pumps, filters, pipes, nets, hooks or mats. Similarly, fish and/or fish eggs could be photodynamically decontaminated. Similarly, terraria, aquarium containers, sand, gravel and/or green plants could be photodynamically decontaminated before and/or during their use.

Examples of suitable electronic equipment include hot plates, remote controls, headphones, hands-free modules, headsets, mobile telephones, or control elements such as buttons, switches, touch screens or keys. Examples of suitable building materials include concrete, glass, sand, gravel, wall claddings, plaster, screed or the like.

Examples of suitable wall claddings include wood paneling, tiles, solid wood panels, medium density fibreboard, plywood panels, multiplex board, fibre-reinforced concrete panels, plasterboard, gypsum fibreboard, and plastic, foam and/or cellulose wallpapers.

As an example, at least one dispersion for use in accordance with the invention may be used to remove mould.

Preferably, a surface coated with mould is treated with at least one dispersion for use in accordance with the invention and subsequently irradiated with a suitable source of radiation which produces electromagnetic radiation of a suitable wavelength and energy density, whereupon a reduction, preferably inactivation, in the mould occurs on the treated surface.

In the said preferred embodiment of the use in accordance with the invention or of the method in accordance with the invention, the irradiation of the microorganisms and of the at least one dispersion for use in accordance with the invention with electromagnetic radiation of a suitable wavelength and energy density is carried out in the presence of at least one oxygen-donating compound, preferably peroxide, and/or at least one oxygen-containing gas, preferably oxygen.

The at least one oxygen-donating compound and/or the at least one oxygen-containing gas may preferably be applied before or during irradiation with electromagnetic radiation of a suitable wavelength and energy density.

By additionally providing oxygen in the form of at least one oxygen-containing compound and/or at least one oxygen-containing gas before or during irradiation of the microorganisms and of the at least one photosensitizer with electromagnetic radiation of a suitable wavelength and energy density, the yield of reactive oxygen species (ROS), preferably oxygen radicals and/or singlet oxygen, is increased.

In accordance with a first aspect, the present invention concerns a photosensitizer dispersion comprising:

(a) at least one photosensitizer,

(b) at least one liquid polar phase, and

(c) at least one surfactant.

In accordance with a second aspect, the present invention concerns a photosensitizer dispersion in accordance with aspect 1, wherein the at least one photosensitizer is positively charged, negatively charged or uncharged, wherein the at least one photosensitizer more preferably comprises at least one organic residue with a) at least one neutral, nitrogen atom which can be protonated and/or b) at least one positively charged nitrogen atom.

In accordance with a third aspect, the present invention concerns a photosensitizer dispersion in accordance with one of aspects 1 or 2, wherein the at least one photosensitizer is selected from the group which consists of phenalenones, curcumins, flavins, porphyrins, porphycenes, xanthene dyes, coumarins, phthalocyanines, phenothiazine compounds, anthracene dyes, pyrenes, fullerenes, perylenes and mixtures thereof, preferably from phenalenones, curcumins, flavins, porphyrins, phthalocyanines, phenothiazine compounds and mixtures thereof, more preferably from phenalenones, curcumins, flavins and mixtures thereof.

In accordance with a fourth aspect, the present invention concerns a photosensitizer dispersion in accordance with one of aspects 1 to 3, wherein the at least one photosensitizer is a phenalenone derivative which is selected from the group which consists of the compounds with formulae (2) to (28) and mixtures thereof:

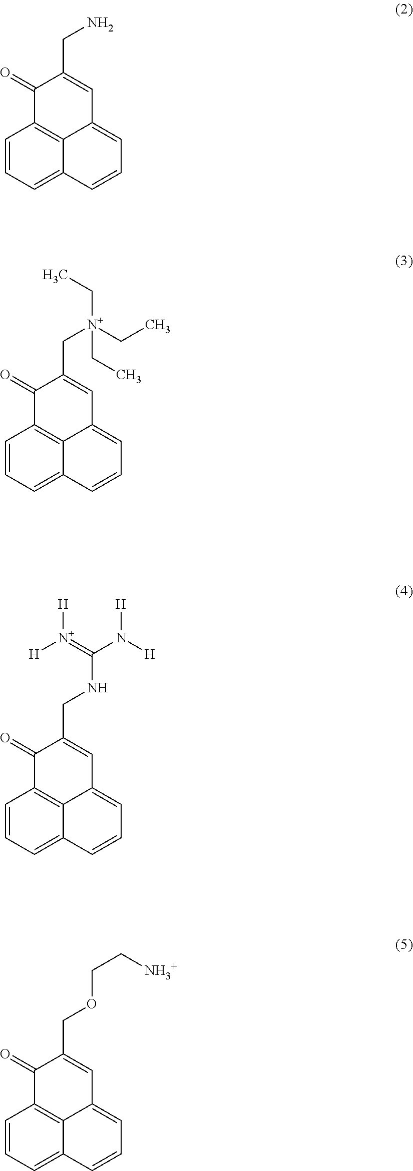 [SMILES: NCC1=Cc2cccc3cccc(c23)C1=O ; CC[N+](CC)(CC)CC1=Cc2cccc3cccc(c23)C1=O ; NC(=[NH2+])NCC1=Cc2cccc3cccc(c23)C1=O ; [NH3+]CCOCC1=Cc2cccc3cccc(c23)C1=O]

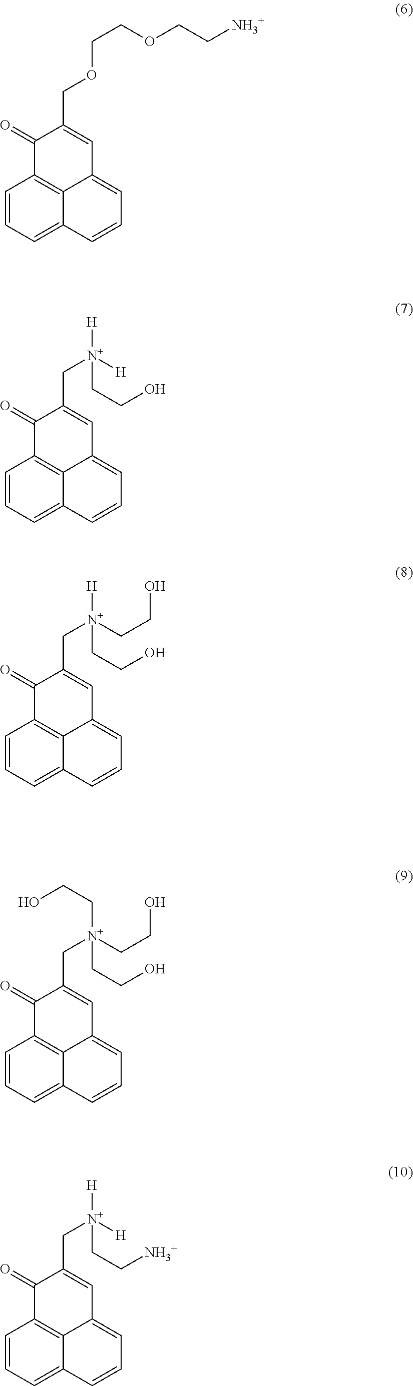 [SMILES: [NH3+]CCOCCOCC1=Cc2cccc3cccc(c23)C1=O ; O=C1C(C[NH2+]CCO)=Cc2cccc3cccc1c23 ; O=C1C(C[NH+](CCO)CCO)=Cc2cccc3cccc1c23 ; O=C1C(C[N+](CCO)(CCO)CCO)=Cc2cccc3cccc1c23 ; [NH3+]CC[NH2+]CC1=Cc2cccc3cccc(c23)C1=O]

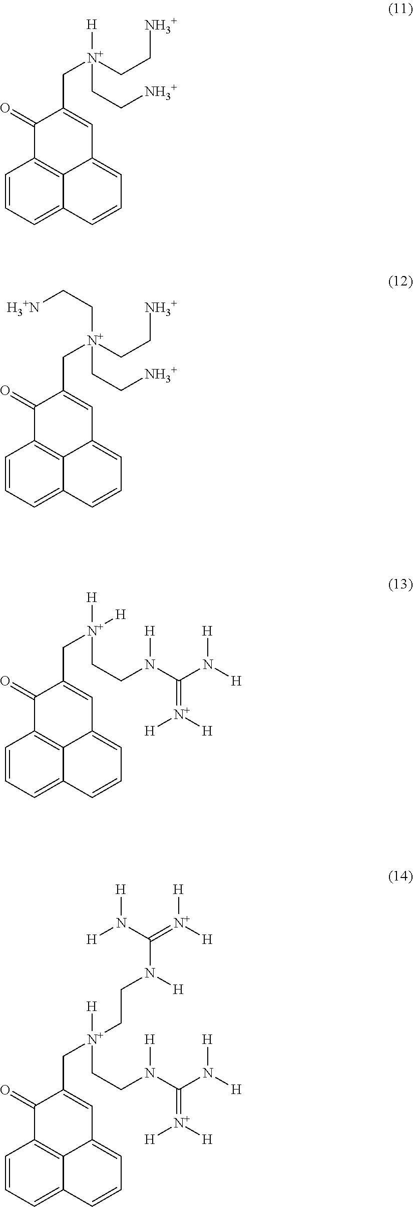

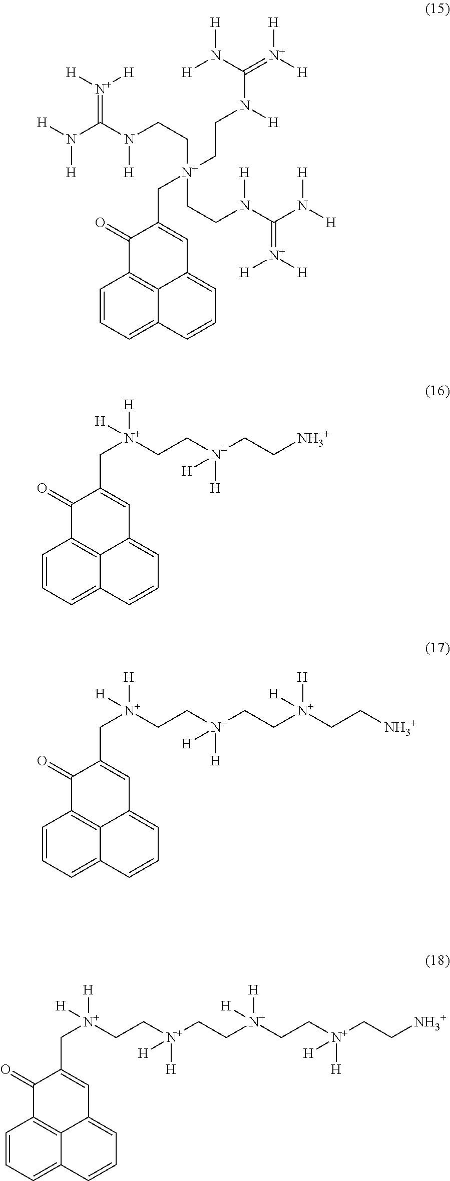 [SMILES: NC(=[NH2+])NCC[N+](CCNC(N)=[NH2+])(CCNC(N)=[NH2+])CC1=Cc2cccc3cccc(c23)C1=O ; [NH3+]CC[NH2+]CC[NH2+]CC1=Cc2cccc3cccc(c23)C1=O ; [NH3+]CC[NH2+]CC[NH2+]CC[NH2+]CC1=Cc2cccc3cccc(c23)C1=O ; [NH3+]CC[NH2+]CC[NH2+]CC[NH2+]CC[NH2+]CC1=Cc2cccc3cccc(c23)C1=O]

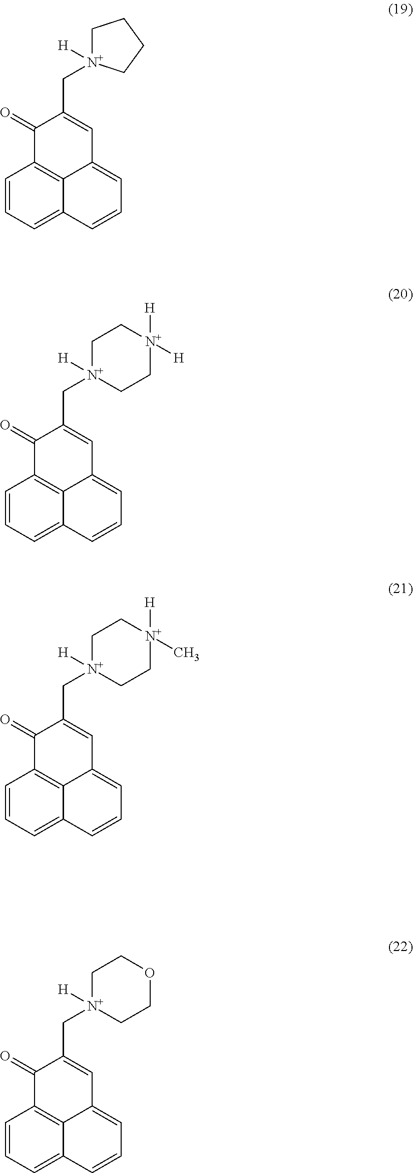 [SMILES: O=C1C(C[NH+]2CCCC2)=Cc2cccc3cccc1c23 ; O=C1C(C[NH+]2CC[NH2+]CC2)=Cc2cccc3cccc1c23 ; C[NH+]1CC[NH+](CC2=Cc3cccc4cccc(c34)C2=O)CC1 ; O=C1C(C[NH+]2CCOCC2)=Cc2cccc3cccc1c23]

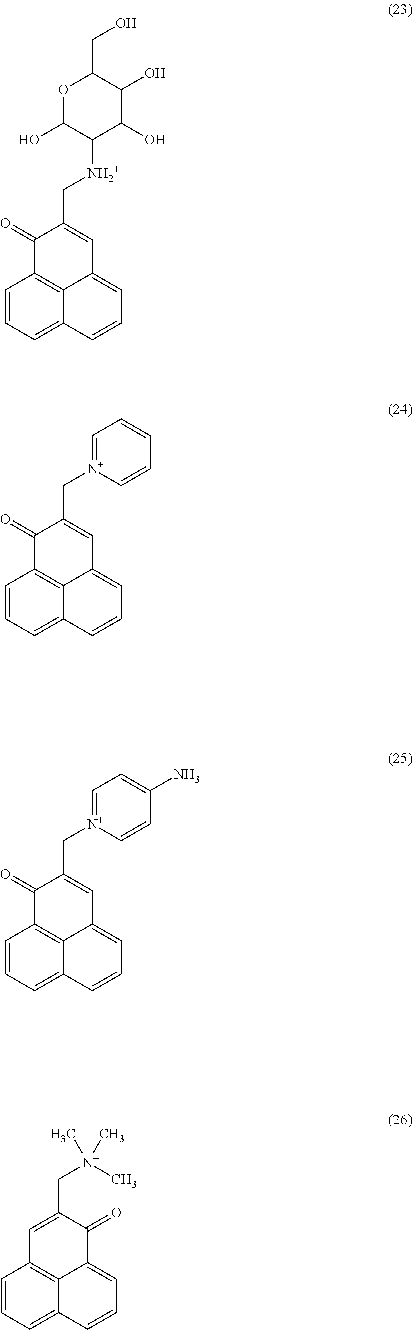 [SMILES: O=C1C(C[NH2+]C2C(O)OC(CO)C(O)C2O)=Cc2cccc3cccc1c23 ; O=C1C(C[n+]2ccccc2)=Cc2cccc3cccc1c23 ; [NH3+]c1cc[n+](CC2=Cc3cccc4cccc(c34)C2=O)cc1 ; C[N+](C)(C)CC1=Cc2cccc3cccc(c23)C1=O]

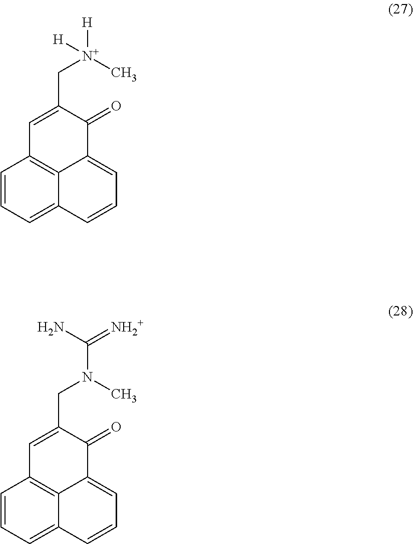 [SMILES: C[NH2+]CC1=Cc2cccc3cccc(c23)C1=O ; CN(CC1=Cc2cccc3cccc(c23)C1=O)C(N)=[NH2+]]

In accordance with a fifth aspect, the present invention concerns a photosensitizer dispersion in accordance with one of aspects 1 to 4, wherein the at least one photosensitizer is a flavin derivative selected from the group which consists of the compound with formulae (32) to (49), (51) to (64) and mixtures thereof:

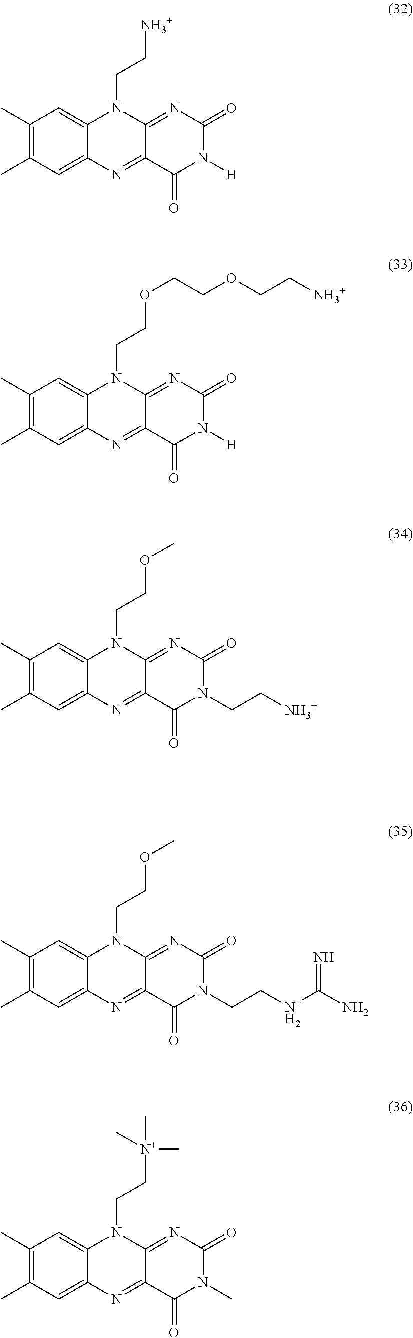

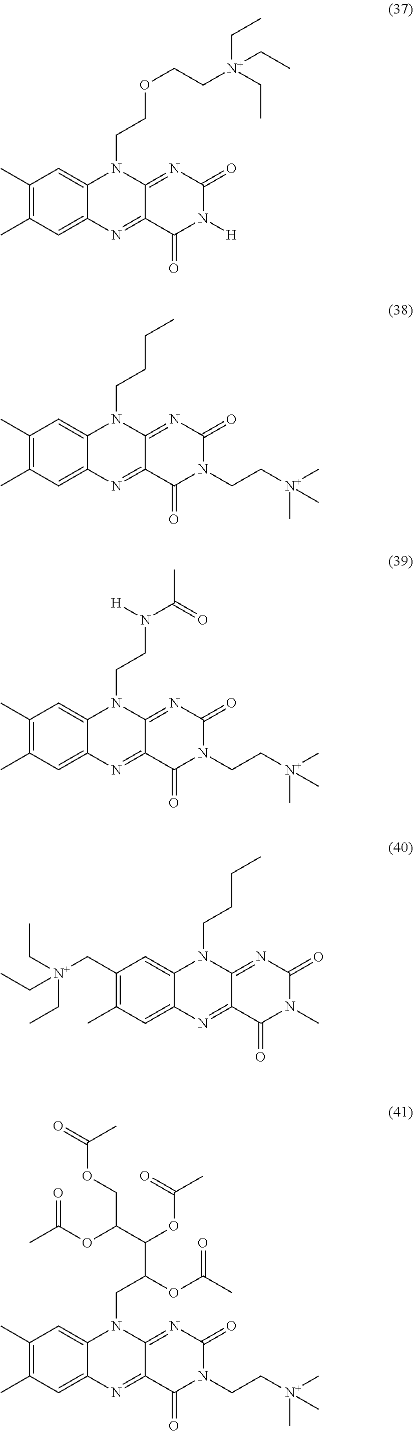

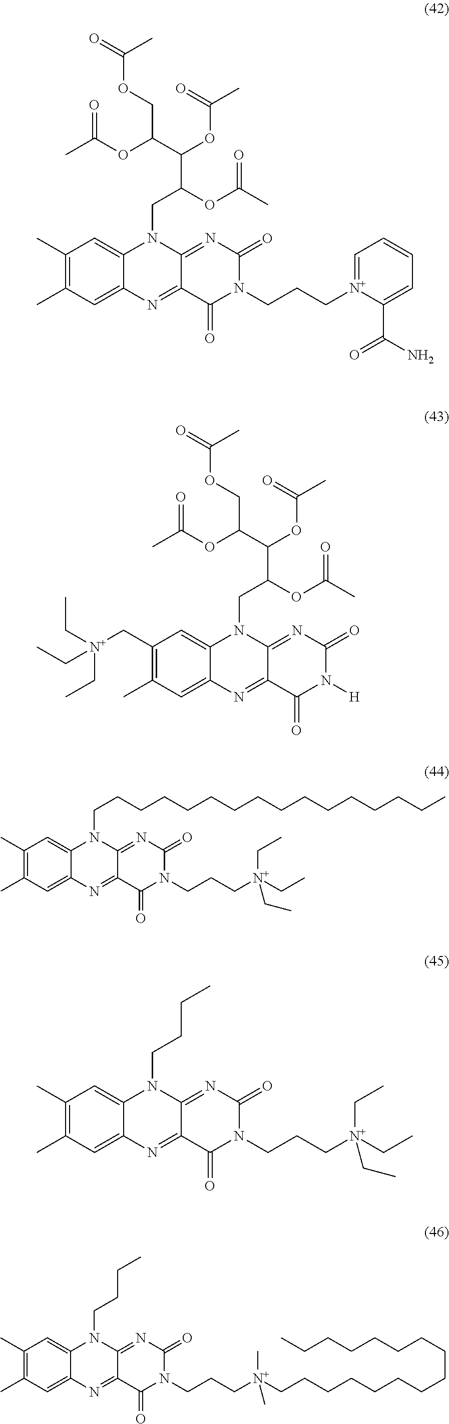

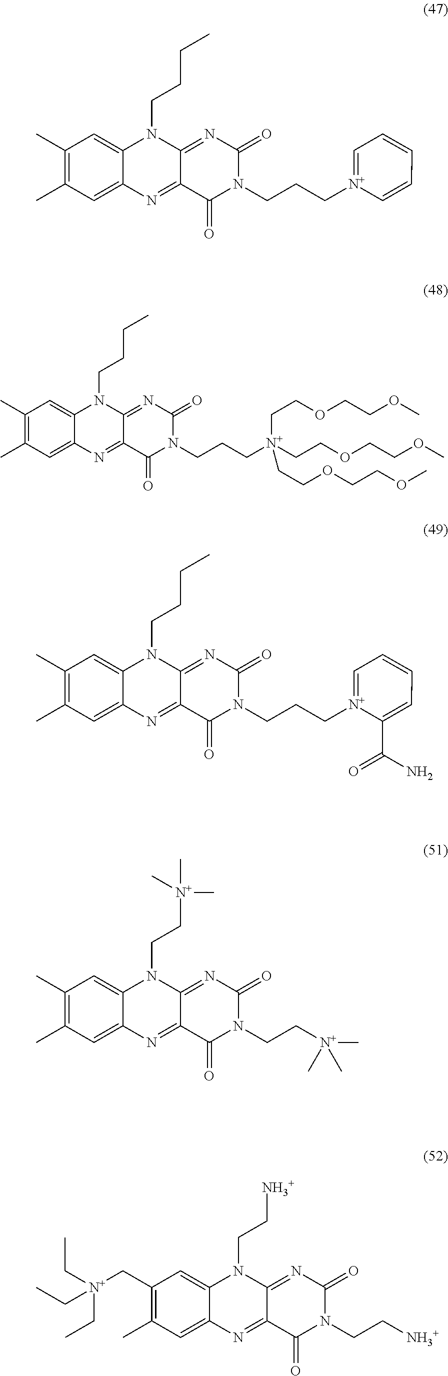

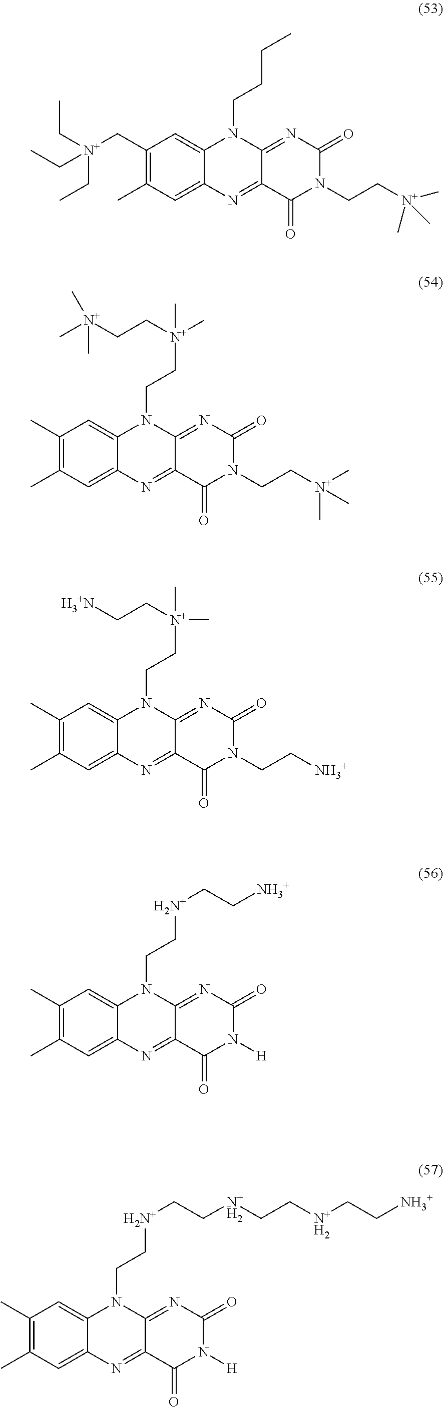

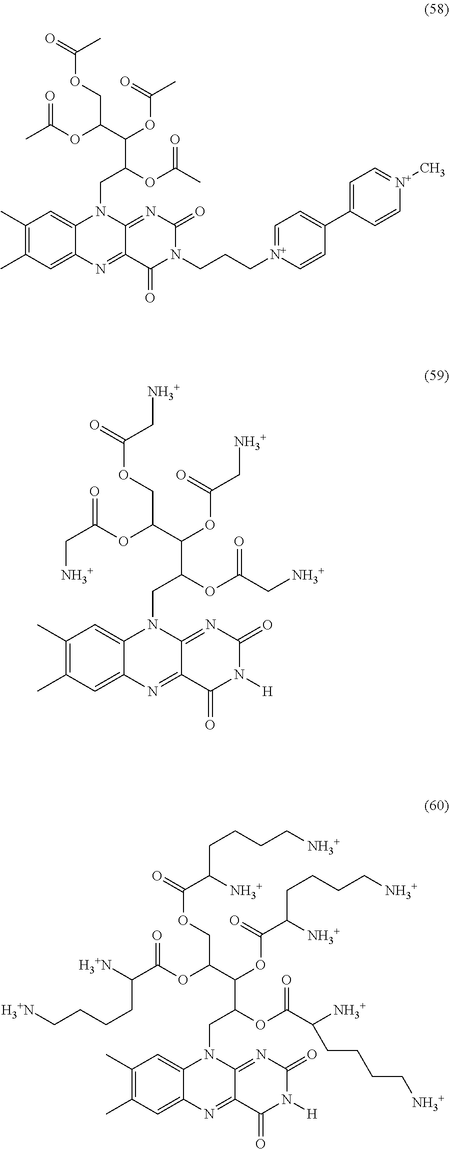

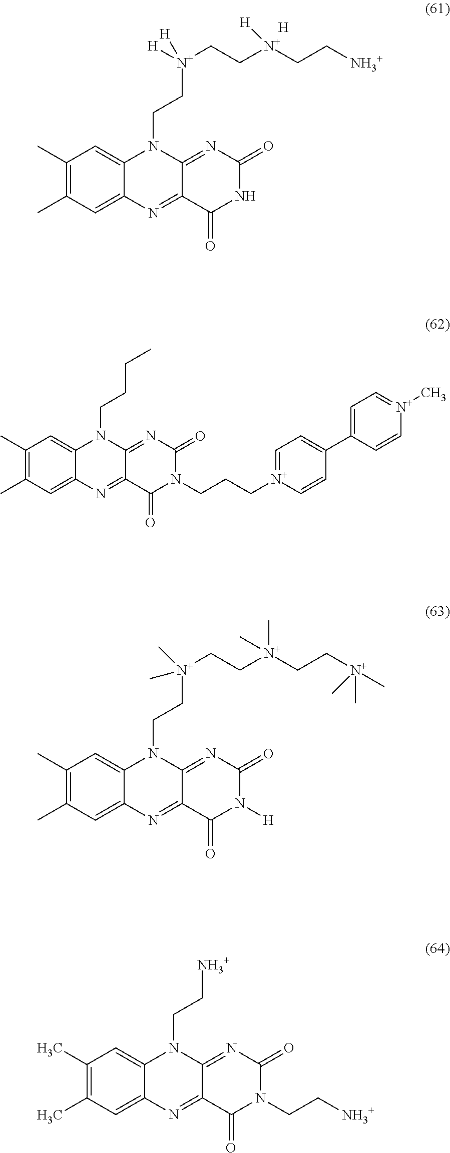

In accordance with a sixth aspect, the present invention concerns a photosensitizer dispersion in accordance with one of aspects 1 to 5, wherein the at least one photosensitizer is a curcumin derivative which is selected from the group which consists of the compounds with formulae (75) to (104b), (105) and mixtures thereof:

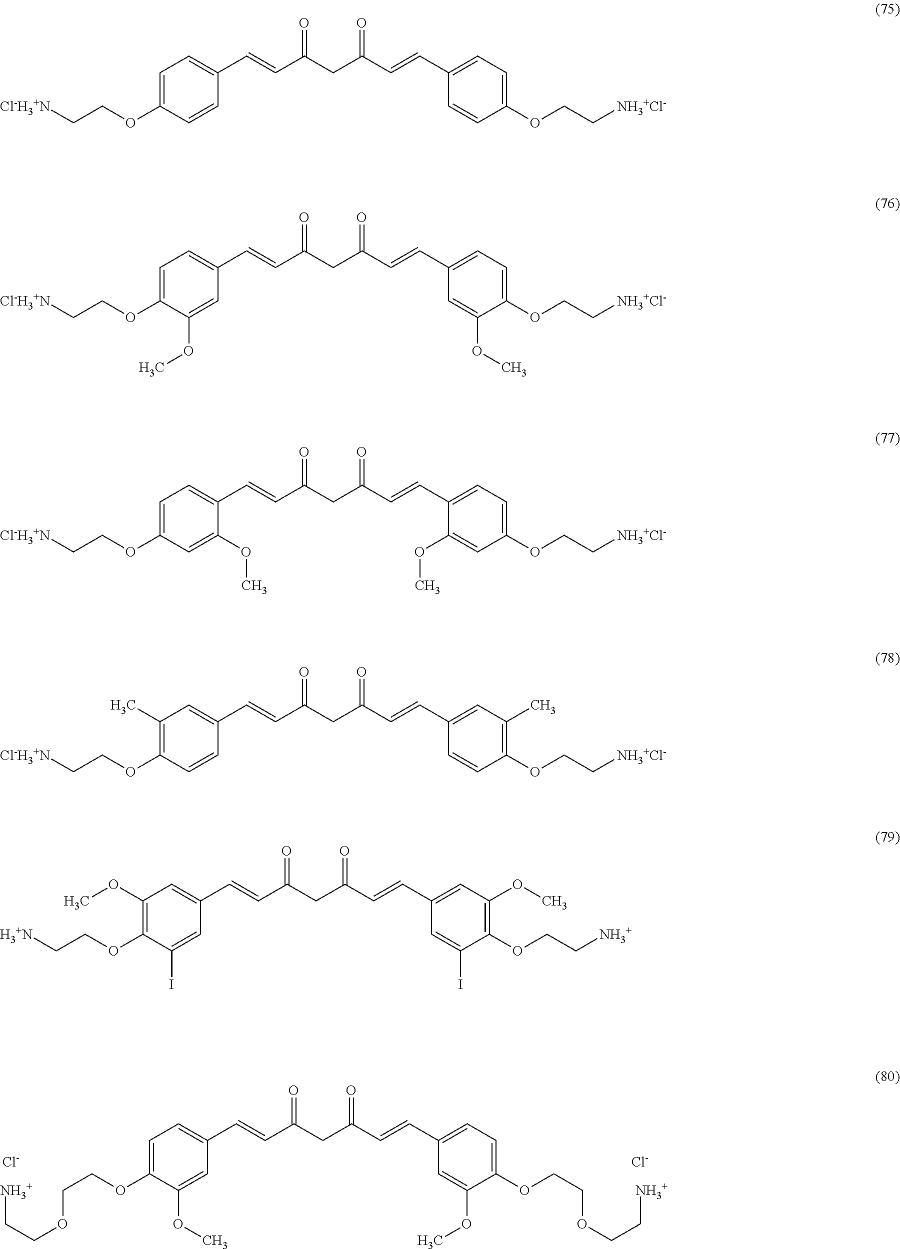

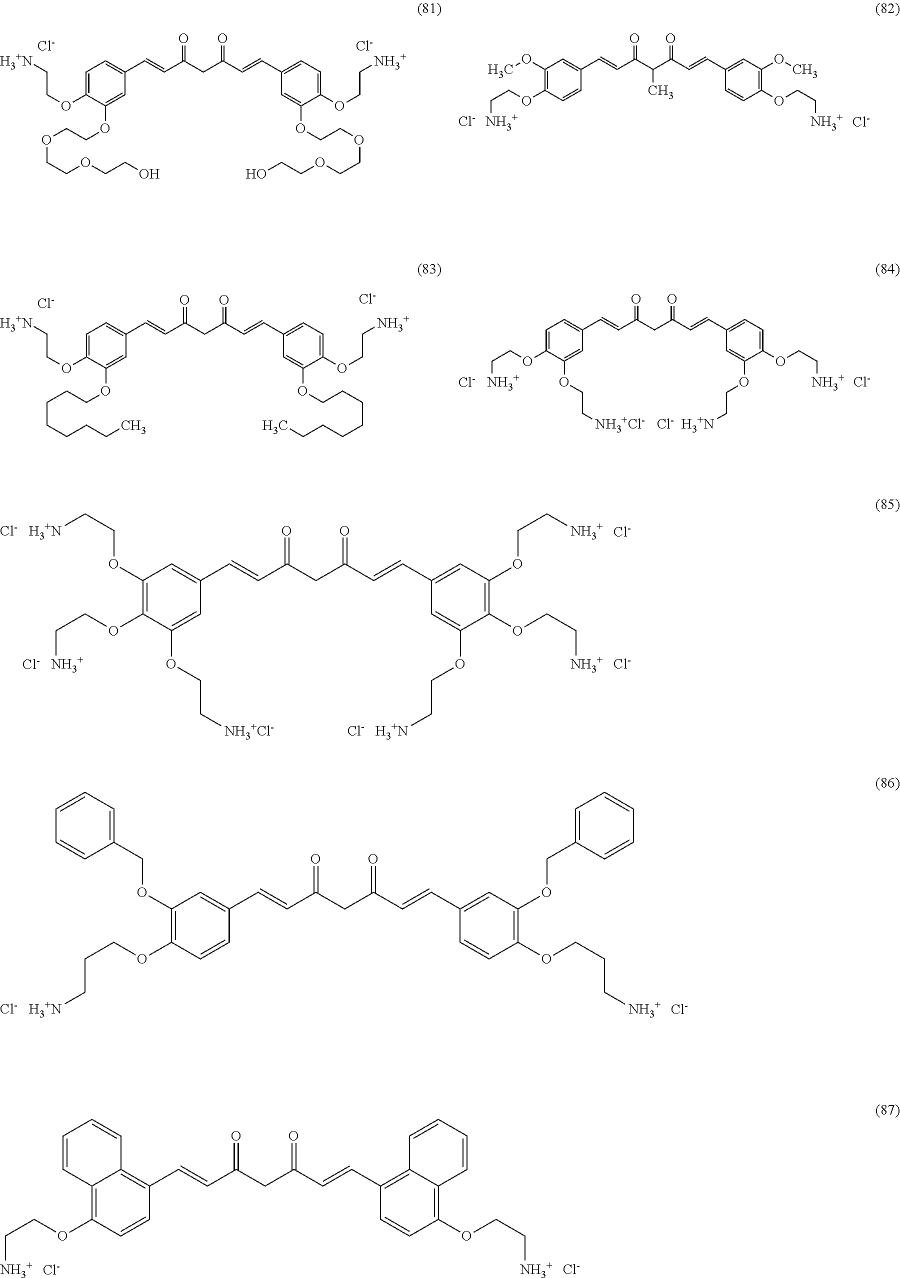

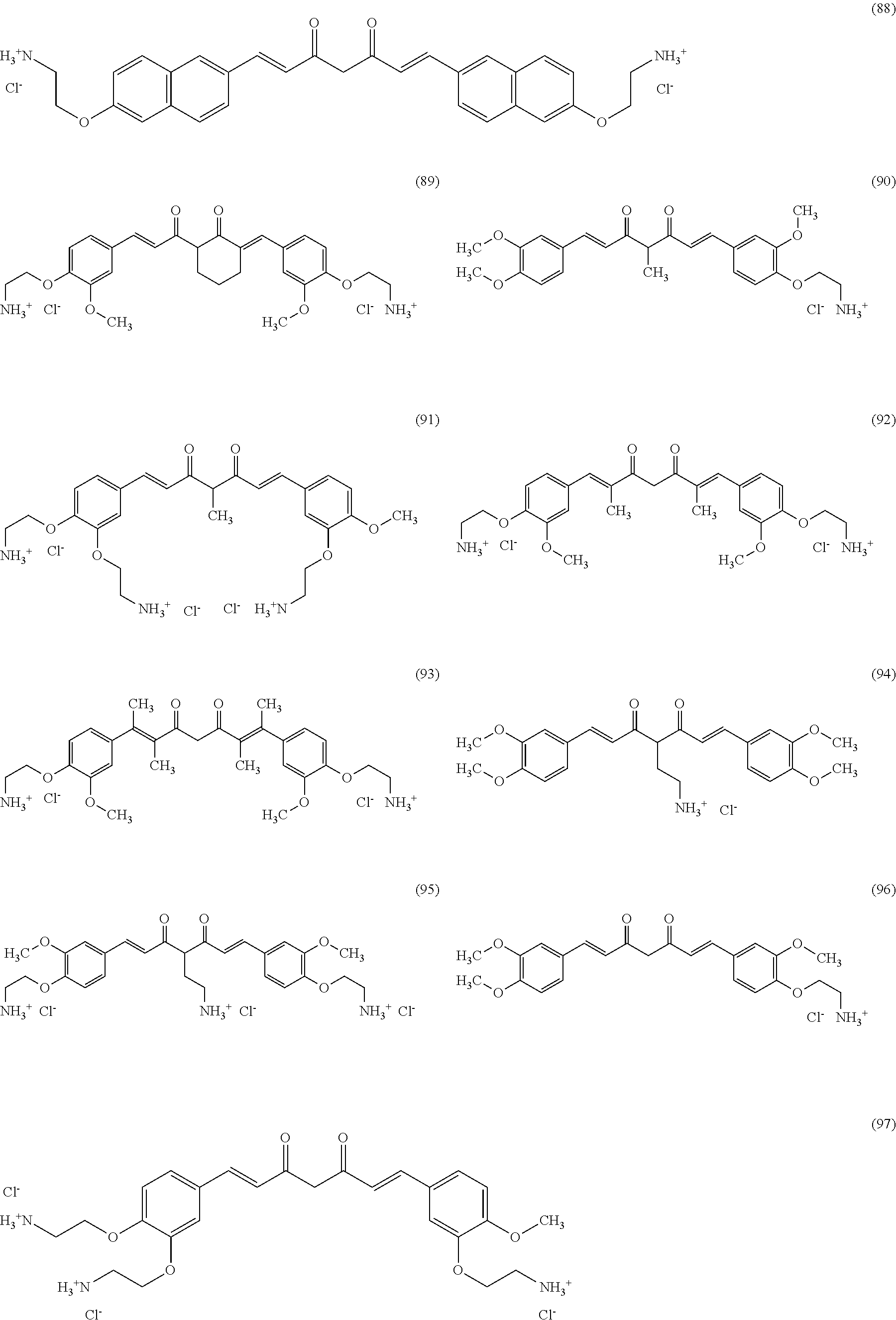

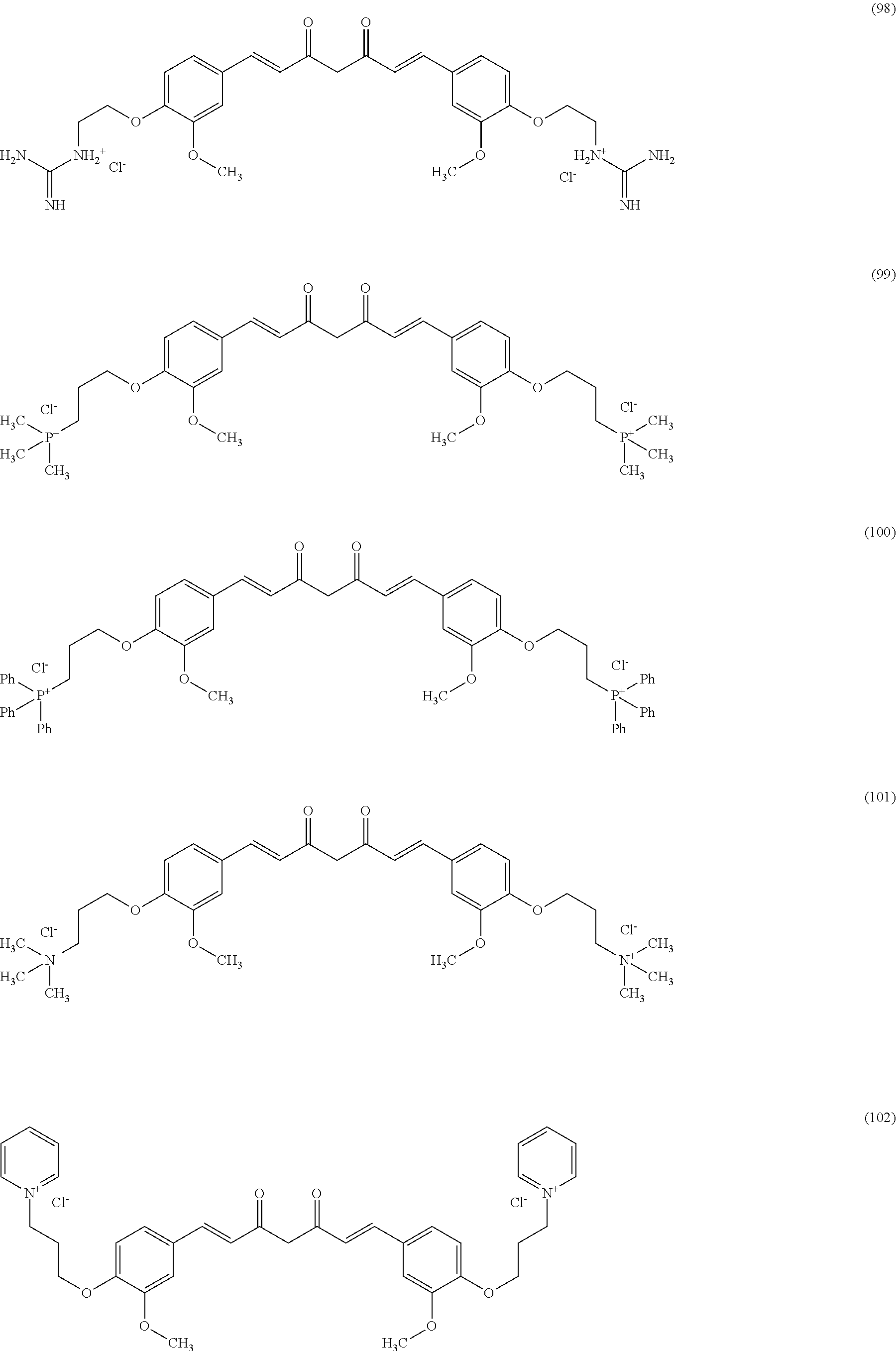

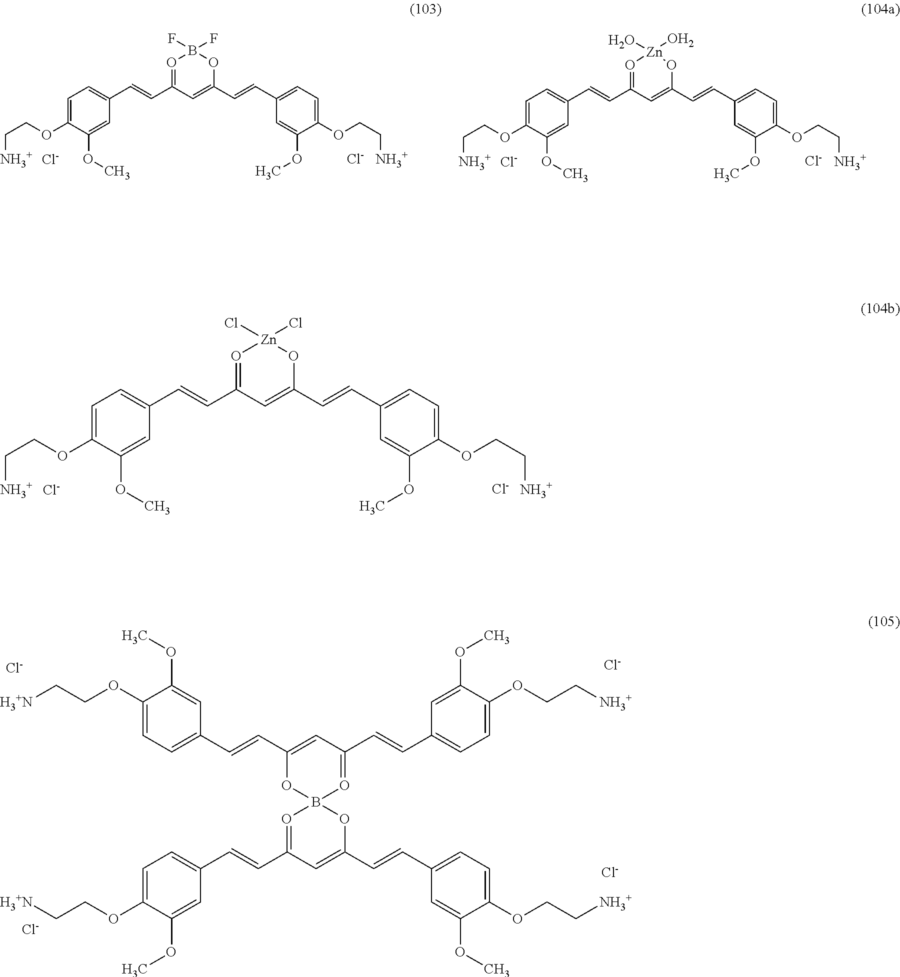

In accordance with a seventh aspect, the present invention concerns a photosensitizer dispersion in accordance with one of aspects 1 to 6, wherein the at least one photosensitizer is selected from the group which consists of the compounds with formulae (2) to (28), (32) to (49), (51) to (64), (75) to (104b), (105) and mixtures thereof.

In accordance with an eighth aspect, the present invention concerns a photosensitizer dispersion in accordance with one of aspects 1 to 7 wherein, as a counter-ion to the positively charged nitrogen atom, at least one anion is selected which is selected from the group which consists of fluoride, chloride, bromide, iodide, sulphate, hydrogen sulphate, phosphate, dihydrogen phosphate, hydrogen phosphate, tosylate, mesylate, formate, acetate, propionate, butanoate, oxalate, tartrate, fumarate, benzoate, citrate and mixtures thereof.

In accordance with a ninth aspect, the present invention concerns a photosensitizer dispersion in accordance with one of aspects 1 to 8, wherein the dispersion comprises the at least one photosensitizer in a concentration in the range 0.1 μM to 1000 μM, preferably in the range 1 μM to 750 μM, more preferably in the range 2 μM to 500 μM.

In accordance with a tenth aspect, the present invention concerns a photosensitizer dispersion in accordance with one of aspects 1 to 9, wherein the at least one liquid polar phase comprises at least one polar solvent, preferably water.

In accordance with an eleventh aspect, the present invention concerns a photosensitizer dispersion in accordance with one of aspects 1 to 10, wherein the dispersion comprises the at least one polar solvent, preferably water, in a proportion of at least 0.1% by weight, preferably at least 0.5% by weight, more preferably at least 1% by weight, more preferably at least 4% by weight, more preferably at least 10% by weight, more preferably at least 35% by weight, more preferably at least 50% by weight, more preferably at least 51% by weight, respectively with respect to the total weight of the dispersion.

In accordance with a twelfth aspect, the present invention concerns a photosensitizer dispersion in accordance with one of aspects 1 to 11, wherein the dispersion comprises the at least one polar solvent, preferably water, in a proportion in the range 0.1% by weight to 99.8% by weight, preferably in the range 0.5% by weight to 99% by weight, more preferably in the range 4% by weight to 98% by weight, more preferably in the range 10% by weight to 97% by weight, more preferably in the range 35% by weight to 96% by weight, more preferably in the range 50% by weight to 95% by weight, more preferably in the range 51% by weight to 94% by weight, more preferably in the range 53% by weight to 93% by weight, more preferably in the range 70% by weight to 92% by weight, respectively with respect to the total weight of the dispersion.

In accordance with a thirteenth aspect, the present invention concerns a photosensitizer dispersion in accordance with one of aspects 1 to 12, wherein the at least one surfactant is selected from the group which consists of the aforementioned non-ionic surfactants, the aforementioned anionic surfactants, the aforementioned cationic surfactants, the aforementioned amphoteric surfactants and mixtures thereof, preferably the aforementioned non-ionic surfactants, the aforementioned anionic surfactants and mixtures thereof.

In accordance with a fourteenth aspect, the present invention concerns a photosensitizer dispersion in accordance with one of aspects 1 to 13, wherein the dispersion comprises the at least one surfactant in a proportion in the range 0.1% by weight to 65% by weight, preferably in the range 1% by weight to 55% by weight, more preferably in the range 3% by weight to 50% by weight, more preferably in the range 5% by weight to 41% by weight, more preferably in the range 7% by weight to 37% by weight, more preferably in the range 9% by weight to 30% by weight, more preferably in the range 10% by weight to 27% by weight, respectively with respect to the total weight of the dispersion.

In accordance with a fifteenth aspect, the present invention concerns a photosensitizer dispersion in accordance with one of aspects 1 to 14, wherein the non-ionic surfactants are selected from the group which consists of the aforementioned polyalkyleneglycol ethers, the aforementioned alkylglucosides, the aforementioned alkylpolyglycosides, the aforementioned alkylglycoside esters and mixtures thereof.

In accordance with a sixteenth aspect, the present invention concerns a photosensitizer dispersion in accordance with one of aspects 1 to 15, wherein the anionic surfactants are selected from the group which consists of the aforementioned alkylcarboxylates, the aforementioned alkylsulphonates, the aforementioned alkylsulphates, the aforementioned alkylphosphates, the aforementioned alkylpolyglycolethersulphates, the aforementioned sulphonates of alkylcarboxylic acid esters, the aforementioned N-alkyl-sarcosinates and mixtures thereof.

In accordance with a seventeenth aspect, the present invention concerns a photosensitizer dispersion in accordance with one of aspects 1 to 16, wherein the cationic surfactants are selected from the group which consists of the aforementioned quaternary alkylammonium salts, the aforementioned esterquats, the aforementioned acylated polyamines, the aforementioned benzylammonium salts and mixtures thereof.

In accordance with an eighteenth aspect, the present invention concerns a photosensitizer dispersion in accordance with one of aspects 1 to 17, wherein the dispersion further comprises at least one liquid non-polar phase which comprises a non-polar solvent which is selected from the group which consists of the aforementioned acyclic alkanes containing 5 to 30 carbon atoms, the aforementioned cyclic alkanes containing 5 to 13 carbon atoms, the aforementioned perfluoroalkanes containing 5 to 20 carbon atoms, the aforementioned monocarboxylic acid esters preferably containing 4 to 20 carbon atoms, the aforementioned polycarboxylic acid esters preferably containing 6 to 20 carbon atoms, and mixtures thereof.

In accordance with a nineteenth aspect, the present invention concerns a photosensitizer dispersion in accordance with one of aspects 1 to 18, wherein the dispersion comprises the at least one non-polar solvent in a proportion of at least 0.1% by weight, preferably at least 0.5% by weight, more preferably at least 1% by weight, more preferably at least 4% by weight, more preferably at least 10% by weight, more preferably at least 35% by weight, more preferably at least 50% by weight, more preferably at least 51% by weight, respectively with respect to the total weight of the dispersion.

In accordance with a twentieth aspect, the present invention concerns a photosensitizer dispersion in accordance with one of aspects 1 to 19, wherein the dispersion comprises the at least one non-polar solvent in a proportion in the range 0.1% by weight to 99.8% by weight, preferably in the range 0.5% by weight to 99% by weight, more preferably in the range 1% by weight to 96% by weight, more preferably in the range 1.5% by weight to 90% by weight, more preferably in the range 3% by weight to 80% by weight, more preferably in the range 5% by weight to 75% by weight, more preferably in the range 10% by weight to 60% by weight, more preferably in the range 12% by weight to 49% by weight, respectively with respect to the total weight of the dispersion.

In accordance with a twenty-first aspect, the present invention concerns a photosensitizer dispersion in accordance with one of aspects 1 to 20, wherein the dispersion further contains at least one alkanol containing 2 to 12 carbon atoms, and preferably containing 1 to 6 OH groups.

In accordance with a twenty-second aspect, the present invention concerns a photosensitizer dispersion in accordance with one of aspects 1 to 21, wherein the dispersion comprises the at least one alkanol in a proportion in the range 0% by weight to 50% by weight, preferably in the range 0.1% by weight to 40% by weight, more preferably in the range 0.5% by weight to 35% by weight, more preferably in the range 1% by weight to 30% by weight, more preferably in the range 1.5% by weight to 25% by weight, more preferably in the range 5% by weight to 20% by weight, more preferably in the range 7% by weight to 19% by weight, more preferably in the range 10% by weight to 17% by weight, respectively with respect to the total weight of the dispersion.

In accordance with a twenty-third aspect, the present invention concerns a photosensitizer dispersion in accordance with one of aspects 1 to 22, wherein the dispersion comprises or is constituted by a microemulsion, preferably an oil-in-water (O/W) microemulsion, a water-in-oil (W/O) microemulsion or a bicontinuous microemulsion, preferably an oil-in-water (O/W) microemulsion or a water-in-oil (W/O) microemulsion, at a pressure in the range 800 to 1200 mbar and a temperature in the range 2° C. to 50° C.

In accordance with a twenty-fourth aspect, the present invention concerns a photosensitizer dispersion in accordance with one of aspects 1 to 23, wherein the dispersion comprises or is a microemulsion, preferably an oil-in-water (O/W) microemulsion, which comprises:
(a) at least one photosensitizer, which is more preferably selected from the group which consists of the aforementioned phenalenones, the aforementioned curcumins, the aforementioned flavins, the aforementioned porphyrins, the aforementioned porphycenes, the aforementioned xanthene dyes, the aforementioned coumarins, the aforementioned phthalocyanines, the aforementioned phenothiazine compounds, the aforementioned anthracene dyes, the aforementioned pyrenes, the aforementioned fullerenes, the aforementioned perylenes and mixtures thereof, preferably from the aforementioned phenalenones, the aforementioned curcumins, the aforementioned flavins, the aforementioned porphyrins, the aforementioned phthalocyanines, the aforementioned phenothiazine compounds and mixtures thereof, more preferably from the aforementioned phenalenones, the aforementioned curcumins, the aforementioned flavins and mixtures thereof, more preferably from the compounds with formulae (2) to (25), (32) to (49), (51) to (64), (75) to (105) and mixtures thereof,
(b) at least one polar solvent, preferably water,
(c) at least one surfactant which is selected from the group which consists of the aforementioned non-ionic surfactants, the aforementioned anionic surfactants, the aforementioned cationic surfactants, the aforementioned amphoteric surfactants and mixtures thereof, preferably the aforementioned non-ionic surfactants, the aforementioned anionic surfactants and mixtures thereof, and
(d) at least one non-polar solvent, which is more preferably selected from the group which consists of the aforementioned acyclic alkanes containing 5 to 30 carbon atoms, the aforementioned cyclic alkanes containing 5 to 13 carbon atoms, the aforementioned perfluoroalkanes containing 5 to 20 carbon atoms, the aforementioned monocarboxylic acid esters containing 4 to 20 carbon atoms, the aforementioned polycarboxylic acid esters containing 6 to 20 carbon atoms and mixtures thereof, and
(e) optionally, at least one alkanol which is selected from the group which consists of the aforementioned alkanols containing 2 to 12 carbon atoms and preferably containing 1 to 6 OH groups, and mixtures thereof.

In accordance with a twenty-fifth aspect, the present invention concerns a photosensitizer dispersion in accordance with one of aspects 1 to 24, wherein the dispersion is an oil-in-water (O/W) microemulsion, which preferably comprises the at least one non-polar solvent in a proportion in the range 0.1% by weight to 49.9% by weight, preferably in the range 0.5% by weight to 48% by weight, more preferably in the range 1% by weight to 45% by weight, more preferably in the range 3% by weight to 40% by weight, more preferably in the range 5% by weight to 35% by weight, more preferably in the range 7% by weight to 30% by weight, respectively with respect to the total weight of the microemulsion, and preferably the at least one polar solvent, preferably water, in a proportion in the range 50% by weight to 99.8% by weight, preferably in the range 51% by weight to 99% by weight, more preferably in the range 52% by weight to 96% by weight, more preferably in the range 53% by weight to 90% by weight, more preferably in the range 54% by weight to 85% by weight, respectively with respect to the total weight of the microemulsion, and preferably the at least one surfactant in a proportion in the range 0.1% by weight to 45% by weight, preferably in the range 0.5% by weight to 40% by weight, more preferably in the range 1% by weight to 35% by weight, more preferably in the range 3% by weight to 30% by weight, more preferably in the range 5% by weight to 27% by weight, more preferably in the range 7% by weight to 25% by weight, more preferably in the range 10% by weight to 20% by weight, respectively with respect to the total weight of the microemulsion, and optionally, furthermore, the at least one alkanol in a proportion in the range 0% by weight to 50% by weight, preferably in the range 0.1% by weight to 40% by weight, more preferably in the range 0.5% by weight to 35% by weight, more preferably in the range 1% by weight to 30% by weight, more preferably in the range 1.5% by weight to 25% by weight, more preferably in the range 5% by weight to 20% by weight, more preferably in the range 7% by weight to 19% by weight, more preferably in the range 10% by weight to 17% by weight, respectively with respect to the total weight of the microemulsion.

In accordance with a twenty-sixth aspect, the present invention concerns a photosensitizer dispersion in accordance with one of aspects 1 to 25, wherein the dispersion furthermore contains at least one pH-regulating substance which is preferably an inorganic acid, an organic acid, an inorganic base, an organic base, a salt thereof or a mixture thereof.

In accordance with a twenty-seventh aspect, the present invention concerns a photosensitizer dispersion in accordance with one of aspects 1 to 26, wherein the dispersion further comprises at least one gelling agent which is selected from the group which consists of the aforementioned carboxyvinyl polymers, the aforementioned polyacrylamides, the aforementioned alginates, the aforementioned cellulose ethers, and mixtures thereof.

In accordance with a twenty-eighth aspect, the present invention concerns a photosensitizer dispersion in accordance with one of aspects 1 to 27, wherein the dispersion comprises or is a gel, preferably a lyogel, at a pressure in the range 800 to 1200 mbar and a temperature in the range 2° C. to 50° C.

In accordance with a twenty-ninth aspect, the present invention concerns a use of a dispersion according to one of aspects 1 to 28 for the photodynamic inactivation of microorganisms which are preferably selected from the group which consists of viruses, archaeae, bacteria, bacterial spores, fungi, fungal spores, protozoa, algae and blood-borne parasites.

In accordance with a thirtieth aspect, the present invention concerns a use in accordance with aspect 29 for the surface cleaning and/or surface coating of an article.

In accordance with a thirty-first aspect, the present invention concerns a use according to one of aspects 29 to 30, for the surface cleaning and/or surface coating of medical products, food packaging, textiles, building materials, electronic devices, furniture or hygiene articles.

In accordance with a thirty-second aspect, the present invention concerns a use according to one of aspects 29 to 31, for the decontamination of liquids.

In accordance with a thirty-third aspect, the present invention concerns a use according to one of aspects 29 to 32, for the decontamination of foodstuffs.

In accordance with a thirty-fourth aspect, the present invention concerns a method for the photodynamic inactivation of microorganisms, which preferably includes viruses, archaeae, bacteria, bacterial spores, fungi, fungal spores, protozoa, algae, blood-borne parasites or combinations thereof, wherein the method comprises the following steps:

(A) bringing the microorganisms into contact with at least one dispersion according to one of aspects 1 to 28, and

(B) irradiating the microorganisms and at least one photosensitizer contained in the dispersion with electromagnetic radiation of a suitable wavelength and energy density.

The invention will now be explained with the aid of the figures and examples, without in any way being limited thereto.

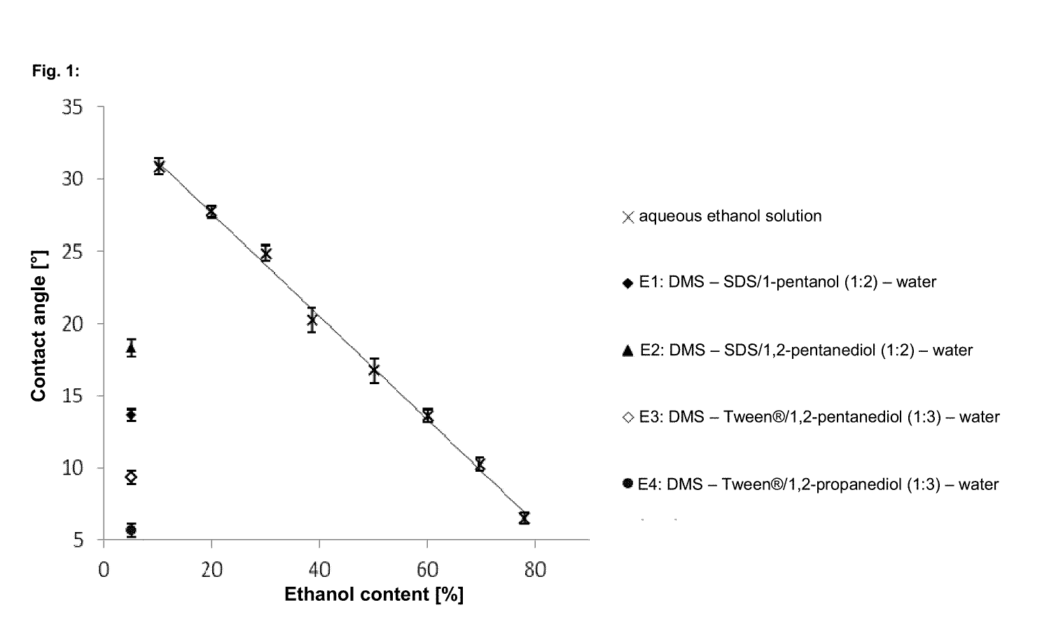

FIG. 1 shows the mean value of the contact angle for the photosensitizer-free microemulsions E1 to E4 as well as aqueous ethanol solutions with the concentrations given.

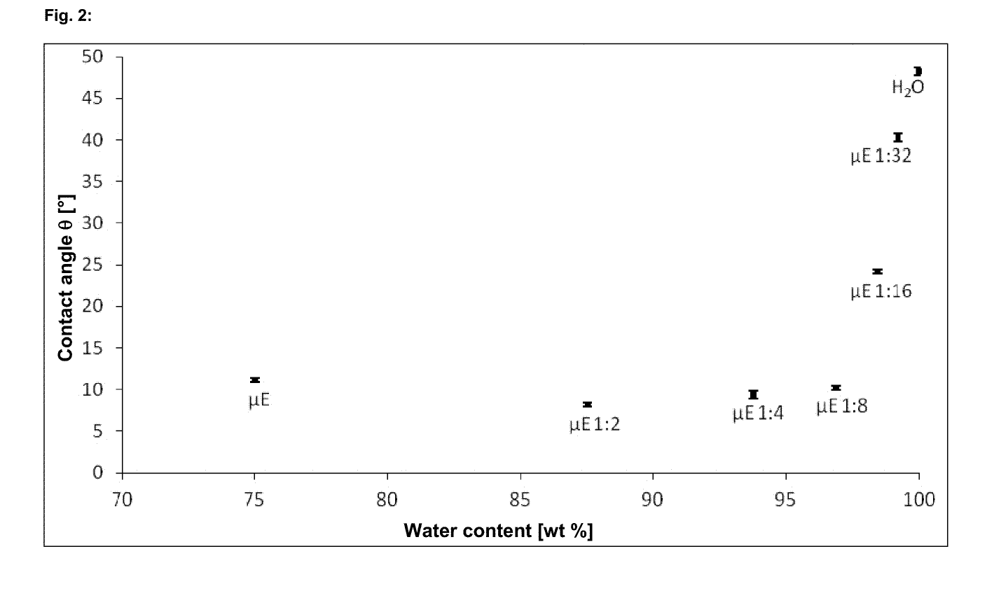

FIG. 2 shows the mean values of the measured contact angle measured in Example 1 of the dilution of a microemulsion E3 (DMS; TWEEN® 20/1,2-pentanediol (1:3); water) with water.

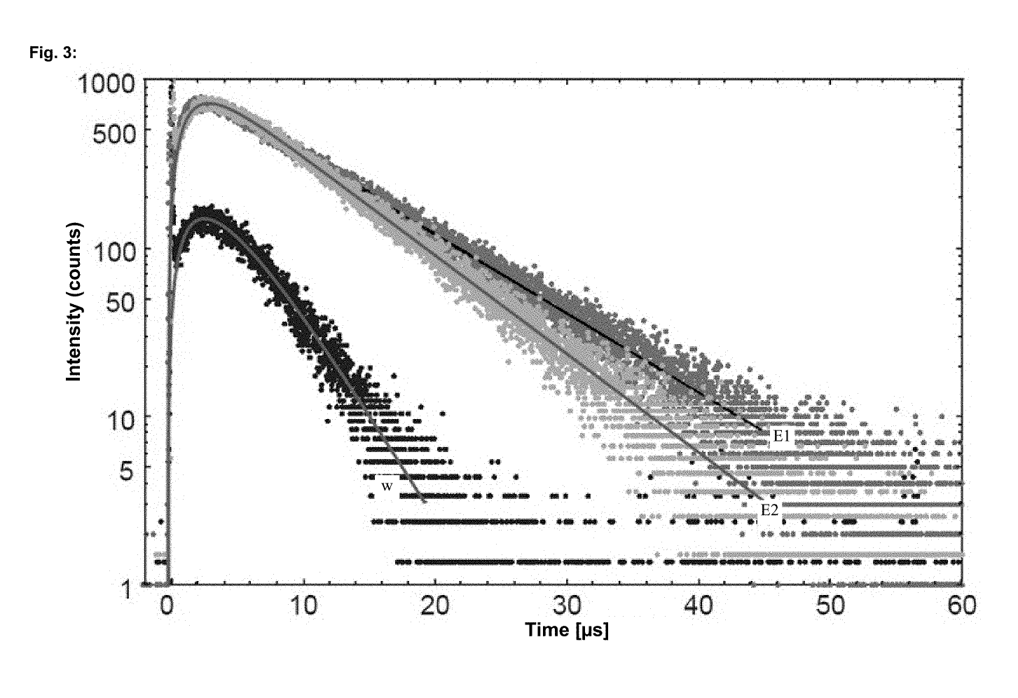

FIG. 3 shows the measured time-resolved singlet oxygen spectra for the photosensitizer TMPyP in water (w), microemulsion E1 (E1) or microemulsion E2 (E2).

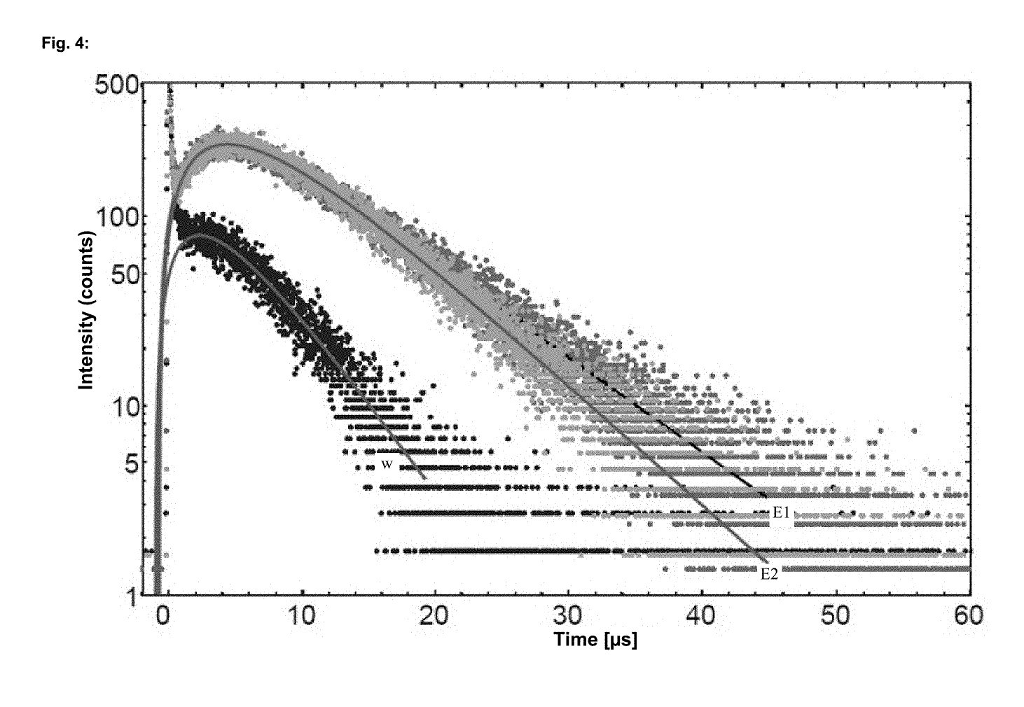

FIG. 4 shows the time-resolved singlet oxygen spectra measured in Example 1 for the photosensitizer SA-PN-01a in water (w), microemulsion E1 (E1) or microemulsion E2 (E2).

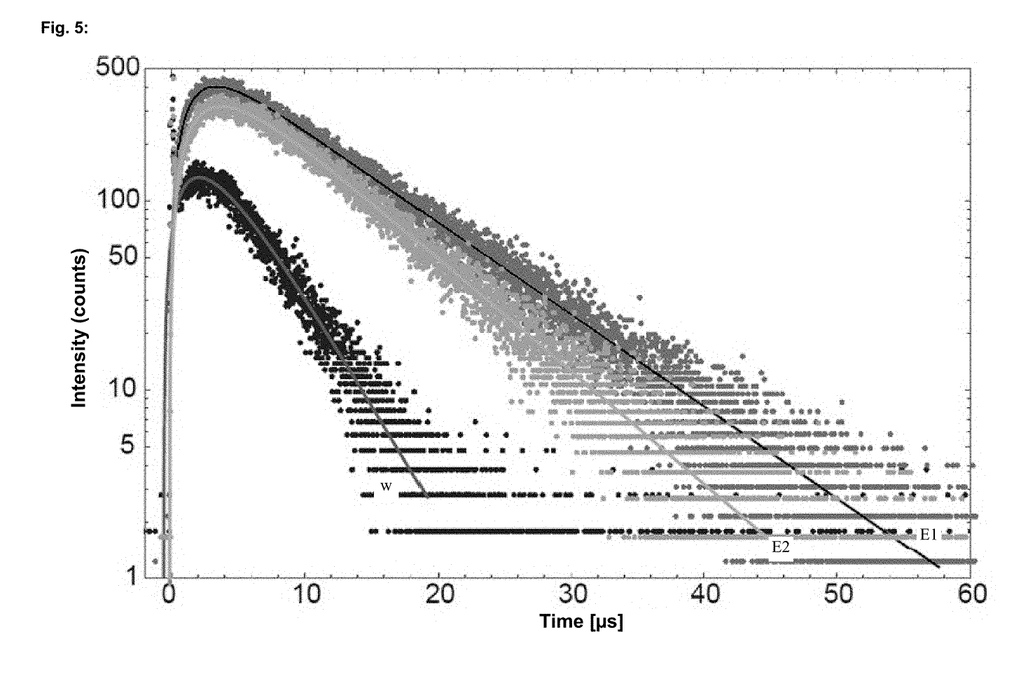

FIG. 5 shows the time-resolved singlet oxygen spectra measured in Example 1 for the photosensitizer FL-AS-H-1a in water (w), microemulsion E1 (E1) or microemulsion E2 (E2).

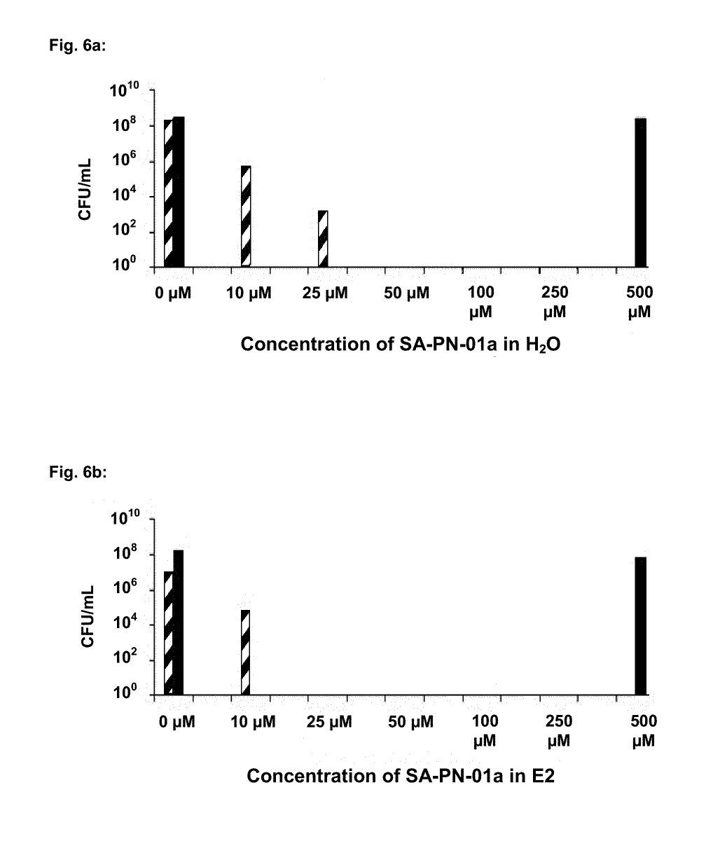

FIG. 6a shows the results measured in Example 1 of the phototoxicity tests for the photosensitizer SA-PN-01a in water in concentrations given.

FIG. 6b shows the results measured in Example 1 of the phototoxicity tests for the photosensitizer SA-PN-01a in microemulsion E2 (E2) in the concentrations given.

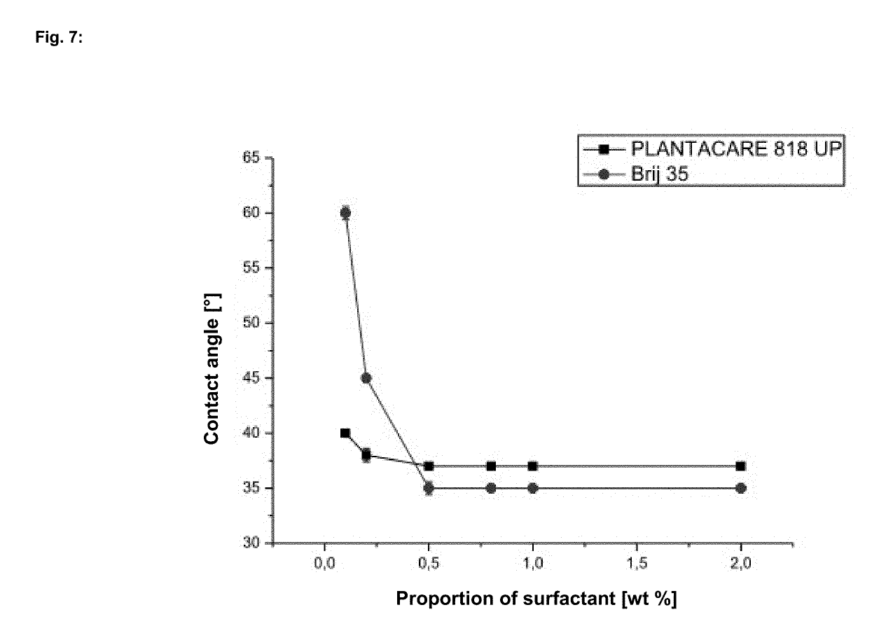

FIG. 7 shows the mean values for the contact angle measured in Example 3 of the photosensitizer-free gels G2 and G3, to which the relevant quantity of the given surfactant had been added.

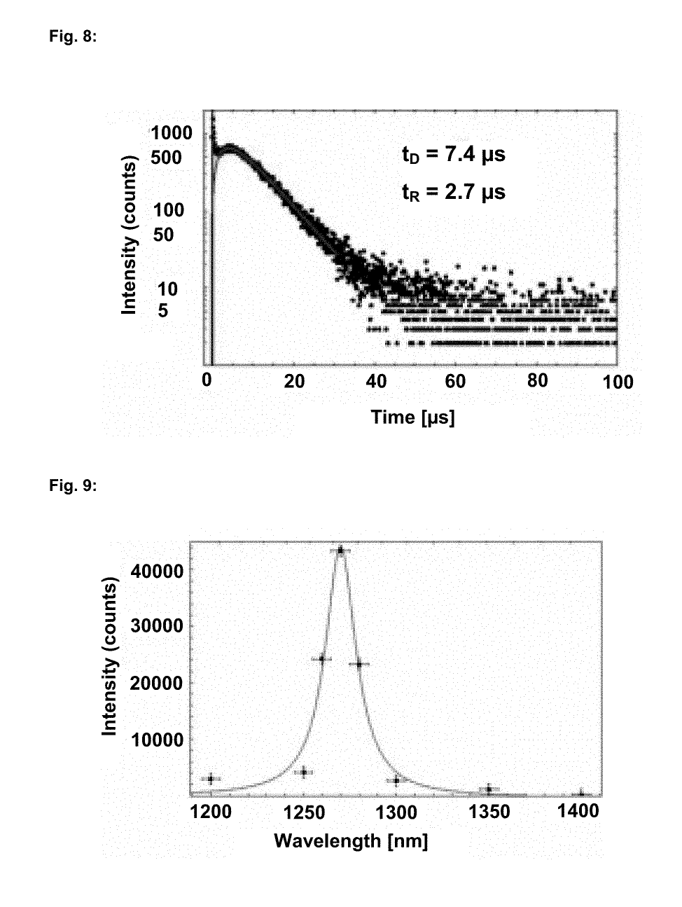

FIG. 8 shows the time-resolved singlet oxygen spectrum measured in Example 3 for the photosensitizer TMPyP in gel G3.

FIG. 9 shows the wavelength-resolved singlet oxygen spectrum measured in Example 3 for the photosensitizer TMPyP in gel G3.

### EXAMPLES

All of the chemicals were purchased from conventional suppliers (TCI, ABCR, Acros, Merck and Fluka) and used without further purification. The solvents were distilled before use and if required, were dried in the normal manner. Dry DMF was purchased from Fluka (Taufkirchen, DE). Thin film chromatography was carried out on thin film aluminium foils coated with silica gel 60 F254, from Merck (Darmstadt, DE). Preparative thin film chromatography was carried out on commercially available glass plates coated with silica gel 60 (20 cm×20 cm, Carl Roth GmbH & Co. KG, Karlsruhe, DE). The compounds were detected with UV light (λ=254 nm, 333 nm) and some detected with the naked eye or stained with ninhydrin. The chromatography was carried out with silica gel (0.060-0.200) from Acros (Waltham, US). NMR spectra were recorded on a Bruker Avance 300 spectrometer (300 MHz [¹H-NMR], 75 MHz [¹³C-NMR]) (Bruker Corporation, Billerica, US). All of the chemical displacements are given in δ [ppm] relative to an external standard (tetramethylsilane, TMS). The coupling constants are respectively given in Hz; characterization of the signals: s=singlet, d=doublet, t=triplet, m=multiplet, dd=doublet of doublets, br=broad. Integration determined the relative number of atoms. The definitive identification of the signals in the carbon spectra was carried out using the DEPT method (pulse angle: 135°). Error limits: 0.01 ppm for ¹H-NMR, 0.1 ppm for ¹³C-NMR and 0.1 Hz for coupling constants. The solvent used is noted for each spectrum. The IR spectra were recorded on a Biorad Excalibur FTS 3000 spectrometer (Bio-Rad Laboratories GmbH, Munich, DE). ES-MS was measured using a ThermoQuest Finnigan TSQ 7000 spectrometer, all of the HR-MS were determined on a ThermoQuest Finnigan MAT 95 (respectively Thermo Fisher Scientific Inc, Waltham, US) spectrometer; argon was used as the ionization gas for FAB ionization (fast atom bombardment). The melting points were determined with the aid of the Buchi SMP-20 melting point instrument (Buchi Labortechnik GmbH, Essen, DE) using a glass capillary. All of the UV/VIS spectra were recorded using a Varian Cary 50 Bio UV/VIS spectrometer; the fluorescence spectra were recorded with a Varian Cary Eclipse spectrometer. The solvents for absorption and emission measurements were purchased in special spectroscopic purity grade from Acros or Baker, or Uvasol from Merck. Millipore water (18 MΩ, Milli Q_{Plus}) was used for all of the measurements.

The following photosensitizers were used in the examples below:

### 1.) 5,10,15,20-tetrakis(1-methyl-4-pyridyl)-porphyrin-tetra-(p-toluenesulphonate) (TMPyP, M=1363.65 g/mol)

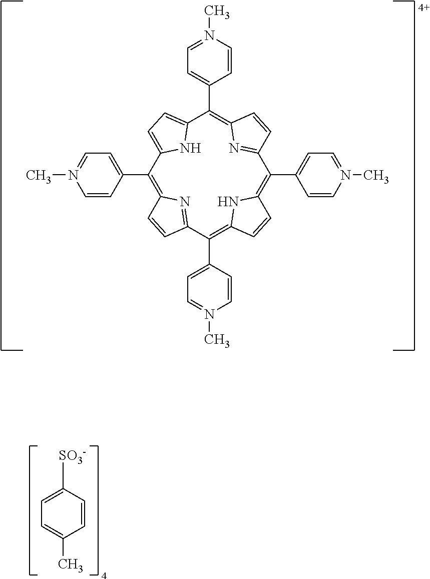

TMPyP was purchased from TCI Germany GmbH (Eschborn, DE).

### 2.) 2-(4-pyridinyl)methyl)-1H-phenalen-1-on-chloride

(SA-PN-01a, M=307.78 g/mol),

Chloride of the compound with formula (24)

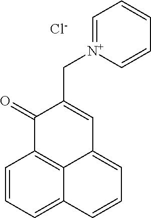 [SMILES: O=C1C(C[n+]2ccccc2)=Cc2cccc3cccc1c23 ; [Cl-]]

SA-PN-01a was produced in accordance with the synthesis described in EP 2 678 035 A2, Example 7. The ¹H-NMR spectrum in DMSO-d6 was identical to the spectrum known from the literature.

### 3a) 10-[2-({[(tert-butyl)oxy]carbonyl}amino)eth-1-yl]-7,8-dimethyl-[3H,10H]-benzo[g]pteridine-2,4-dione (Flavin 32a)

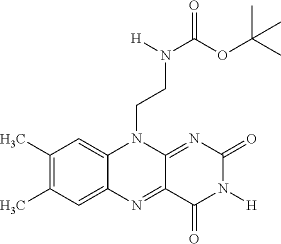 [SMILES: Cc1cc2nc3c(=O)[nH]c(=O)nc-3n(CCNC(=O)OC(C)(C)C)c2cc1C]

The synthesis was carried out as published by Butenandt, J. et al. (2002) using commercially available precursors. The ¹H-NMR spectrum in DMSO-d6 was identical to the spectrum known from the literature.

### 3b) 10-(2-aminoeth-1-yl)-7,8-dimethyl-[3H,10H]-benzo[g]pteridine-2,4-dione hydrochloride (FL-AS-H-1a; M=321.77 g/mol)

Chloride of the Compound with Formula (32)

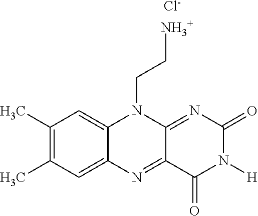 [SMILES: [Cl-] ; Cc1cc2nc3c(=O)[nH]c(=O)nc-3n(CC[NH3+])c2cc1C]

Flavin 32a (2.0 mmol) was dissolved in dichloromethane (100 mL); HCl in diethyl ether (10 mL) was added dropwise and the reaction mixture was stirred overnight in the dark with the exclusion of moisture. The precipitate was aspirated off, washed with diethyl ether and dried. The ¹H-NMR spectrum in DMSO-d6 was identical to the spectrum known from the literature.

### 4a) 3,10-bis[2′-(tert-butyloxycarbonylamino)eth-1′-yl]-7,8-dimethylbenzo[g]-pteridine-2,4-dione (Flavin 64a)

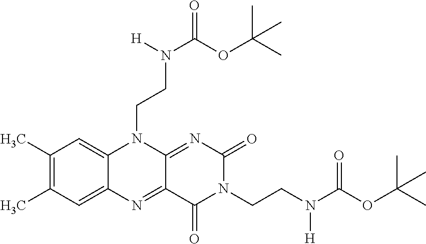 [SMILES: Cc1cc2nc3c(=O)n(CCNC(=O)OC(C)(C)C)c(=O)nc-3n(CCNC(=O)OC(C)(C)C)c2cc1C]

The synthesis of flavin 64a was carried out as described in the publication by Svoboda J. et al. (2008) using flavin 32a. The ¹H-NMR spectrum in DMSO-d6 was identical to the spectrum known from the literature.

### 4b) 3,10-bis(2′-aminoeth-1′-yl)-7,8-dimethylbenzo[g]pteridine-2,4-dion-dihydrochloride (FL-AS-H-2; M=401.29 g/mol)

Dichloride of Compound (64)

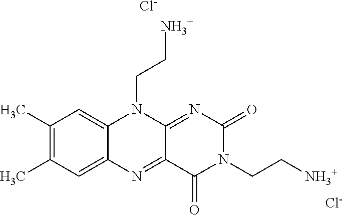 [SMILES: [Cl-] ; Cc1cc2nc3c(=O)n(CC[NH3+])c(=O)nc-3n(CC[NH3+])c2cc1C ; [Cl-]]

Flavin 64a (2.0 mmol) was dissolved in dichloromethane (100 mL); HCl in diethyl ether (10 mL) was added dropwise and the reaction mixture was stirred overnight in the dark with the exclusion of moisture. The precipitate was aspirated off, washed with diethyl ether and dried. The ¹H-NMR spectrum in DMSO-d6 was identical to the spectrum known from the literature.

### 5) Synthesis of Compounds with Formula (26), (27), (28a) and (28)

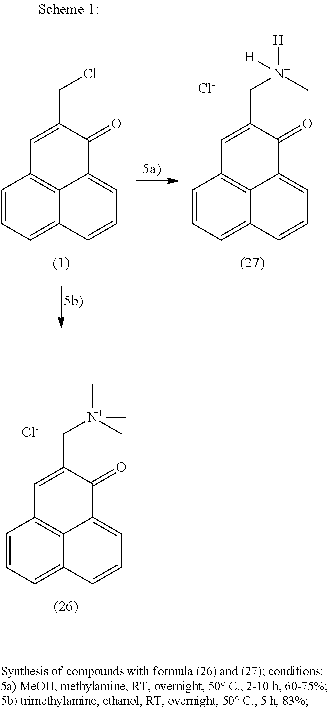 [SMILES: O=C1C(CCl)=Cc2cccc3cccc1c23 ; C[NH2+]CC1=Cc2cccc3cccc(c23)C1=O ; [Cl-] ; C[N+](C)(C)CC1=Cc2cccc3cccc(c23)C1=O ; [Cl-]]

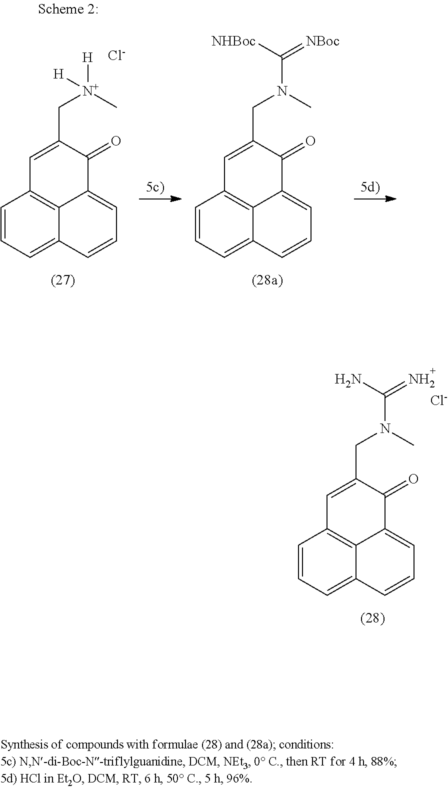

### 5a) N-methyl-N-(1-oxo-1H-phenalen-2-yl)methanaminium Chloride Chloride of the Compound with Formula (27)

An ice-cold solution of methylamine in methanol (40 mL, 10%) was added dropwise over 1 h to 2-chloromethyl-1H-phenalen-1-one (1) (113 mg, 0.5 mmol) in methanol (10 mL). After stirring for 30 h at room temperature, the excess amine and the solvent were driven off in a stream of nitrogen. The residue was dissolved in 4:1 dichloromethane (DCM)/ethanol and precipitated by adding diethyl ether. The product was centrifuged (60 min, 4400 rpm, 0° C.) and the supernatant was discarded. This step was repeated once more. The residue was suspended in diethyl ether. After the yellow solid had settled out, the supernatant was decanted off and discarded. This step was repeated twice more. The product (101 mg, 0.39 mmol) was a yellowish-brown powder.

¹H-NMR (300 MHz, CDCl₃): δ[ppm]=8.66 (d, J=7.4 Hz, 1H), 8.28-8.20 (m, 2H), 8.08 (d, J=8.3 Hz, 1H), 7.94 (d, J=7.0 Hz, 1H), 7.80 (t, J=7.7 Hz, 1H), 7.67-7.59 (m, 1H), 4.20 (s, 2H), 2.79 (s, 3H). —MS (ESI-MS, CH₂Cl₂/MeOH+10 mmol NH₄OAc): e/z (%)=224.1 (MH⁺, 100%); —molecular weight (MW)=224.28+35.45 g/mol; —empirical formula (MF)=C₁₅H₁₄NOCl.

### 5b) N,N,N-trimethyl-1-(1-oxo-1H-phenalen-2-yl)methanaminium chloride (SA-PN-02a)

Chloride of the compound with formula (26)

2-(chloromethyl)-1H-phenalen-1-on (1) (230 mg, 1 mmol) in ethanol (60 mL) was placed in a Schlenk flask. Trimethylamine in ethanol (5 mL, 5.6 M, 23 mmol) was added via the septum using a syringe. The solution was stirred overnight in the dark. Stirring was then continued at 50° C. for 30 h. The solvent volume was reduced to 3 mL. Diethyl ether (50 mL) was added in order to completely precipitate the product. The product was centrifuged (60 min, 4400 rpm, 0° C.) and the supernatant was discarded. The residue was suspended in diethyl ether. After the yellow solid had settled out, the supernatant was decanted off and discarded. This step was repeated twice more. The solid was dried under reduced pressure and a yellow powder was obtained (210 mg, 0.73 mmol).

¹H-NMR (600 MHz, D₂O): δ[ppm]=8.02 (d, J=8 Hz, 1H), 7.97 (d, J=6.3 Hz, 1H), 7.92 (d, J=8.2 Hz, 1H), 7.77 (s, 1H), 7.62 (d, J=7 Hz, 1H), 7.50 (t, J=7.8 Hz, 1H) 7.45 (t, J=7.8 Hz, 1H), 4.12 (s, 2H), 2.98 (s, 9H). —MS (ESI-MS, CH₂Cl₂/MeOH+10 mmol NH₄OAc): e/z (%)=252.1 (100, M+); —MW=287.79 g/mol; —MF=C₁₇H18NOCl;

### 5c) 1-((1-oxo-1H-phenalen-2-yl)methyl)-1-methyl-2,3-di(tert-butoxycarbonyl)guanidine

Compound with Formula (28a)

N,N′-di-Boc-N″-triflylguanidine (0.41 g, 1.05 mmol) in dichloromethane (10 mL) was placed in a dry 25 mL round bottom flask. Triethylamine (0.3 g, 0.39 mL, 3 mmol) was slowly added at 2-5° C. with the exclusion of moisture. Compound 3 (130 mg, 0.5 mmol) was added all at once. After stirring for 5 h at room temperature, it was diluted with dichloromethane (30 mL) and the solution was transferred into a separating funnel. The organic phase was washed with aqueous potassium hydrogen sulphate (10 mL, 5%), saturated sodium bicarbonate solution (10 mL) and saturated sodium chloride solution (20 mL), dried over MgSO₄, filtered and rotary evaporated. The crude product was purified by column chromatography using 1:2 acetone/petroleum ether and the product was obtained as a yellow solid (0.21 g). To purify it further, the material was dissolved in acetone (1 mL) and precipitated with petroleum ether (14 mL). The precipitate was aspirated off and washed with petroleum ether.

¹H-NMR (300 MHz, CDCl₃): δ[ppm]=8.63 (d, J=7.3 Hz, 1H), 8.21 (d, J=7.9 Hz, 1H), 8.03 (d, J=8.2 Hz, 1H), 7.85-7.70 (m, 3H), 7.67-7.54 (m, 1H), 4.59 (s, 2H), 3.01 (s, 3H), 1.50 (s, 9H), 1.48 (s, 9H). —MS (ESI-MS, CH₂Cl₂/MeOH+10 mmol NH₄OAc): e/z (%)=466.1 (MH⁺, 100%); —MW=465.53 g/mol; —MF=C₂₆H₃₁N₃O₅

### 5d) 1-((1-oxo-1H-phenalen-2-yl)methyl)-1-methylguanidinium chloride

(SA-PN-24d)

Chloride of the Compound with Formula (28)

The compound was produced and purified, protected from light. Compound 5 (200 mg, 0.45 mmol) was placed in dichloromethane (20 mL, dried over CaCl₂). A saturated solution of HCl in diethyl ether (2 mL) was added dropwise. After stirring for 4 h at room temperature with the exclusion of moisture, the solution was distributed into two Blue Caps and each filled with diethyl ether to 15 mL. The product was centrifuged (60 min, 4400 rpm, 0° C.) and the supernatant was discarded. The residue was suspended in diethyl ether. After the yellow solid had settled out, the supernatant was decanted off and discarded. This step was repeated twice more. Next, the product was dried under reduced pressure in order to obtain 130 mg of a yellow powder.

¹H-NMR (300 MHz, DMSO-d6): δ[ppm]=8.60-8.47 (m, 4H), 8.33-8.24 (m, 2H), 8.16-8.09 (m, 2H), 7.98-7.89 (m, 2H), 7.84-7.73 (m, 4H), 7.57-7.48 (m, 7H), 4.55-4.42 (m, 4H), 3.05 (s, 6H). —MS (ESI-MS, CH₂Cl₂/MeOH+10 mmol NH₄OAc): e/z (%)=266.1 (MH⁺, 100%); —MW=266.3+35.45=301.75 g/mol; —MF=C₁₆H₁₆N₃OCl

### Example 1

A) Production of Various Water-Containing Microemulsions

The % by weight of the components of the microemulsions E1 to E4 given below are with respect to the total weight of the relevant microemulsion without photosensitizer.

Microemulsion E1: microemulsion consisting of DMS, SDS and 1-pentanol with a constant weight ratio of SDS to 1-pentanol of 1:2, as well as water.

20.0% by weight dimethylsuccinate (DMS)

8.33% by weight sodium dodecylsuiphate (SDS)

16.67% by weight 1-pentanol

55.0% by weight water

Microemulsion E2: microemulsion consisting of DMS, SDS and 1,2-pentanediol with a constant weight ratio of 1:2 SDS to 1,2-pentanediol, as well as water.

20.0% by weight dimethylsuccinate (DMS)

8.33% by weight sodium dodecylsuiphate (SDS)

16.67% by weight 1,2-pentanediol

55.0% by weight water

Microemulsion E3: microemulsion consisting of DMS, TWEEN® 20 and 1,2-pentanediol with a constant weight ratio of TWEEN® 20 to 1,2-pentanediol of 1:3, as well as water.

10.0% by weight dimethylsuccinate (DMS)

3.75% by weight TWEEN® 20

11.25% by weight 1,2-pentanediol

75.0% by weight water

Microemulsion E4: microemulsion consisting of DMS, TWEEN® 20 and 1,2-propanediol with a constant weight ratio of TWEEN® 20 to 1,2-propanediol of 1:3, as well as water

10.0% by weight dimethylsuccinate (DMS)

3.75% by weight TWEEN® 20

11.25% by weight 1,2-propanediol

75.0% by weight water

The relevant microemulsions E1 to E4 were initially produced without photosensitizer, wherein all of the components were measured without water and then mixed together one after the other. After a homogeneous mixture had been obtained, the appropriate quantity of water was added, with constant stirring.

As an example, 100 g of microemulsion E4 was produced by weighing out 3.75 g of TWEEN® 20, 11.25 g of 1,2-propanediol and 10 g of DMS. The resulting solution was stirred until a homogeneous mixture had been obtained. Next, 75 g of water was added, with stirring.

For the further experiments, the photosensitizers were dissolved in the appropriate concentration in the respective microemulsion and stirred until the photosensitizer had been completely dissolved.

B) Contact Angle Test

Wetting of the surfaces by the microemulsions used was determined with the aid of the contact angle test.

For the contact angle test, the emulsions given above were used without photosensitizer, as well as photosensitizer-containing emulsions which contained the photosensitizers TMPyP, SA-PN-01a, SA-PN-02a, SA-PN-24d, FL-AS-H-1a or FL-AS-H-2.

In order to compare the novel dispersions with conventional, alcohol-containing disinfecting solutions, furthermore, aqueous ethanol solutions with various ethanol concentrations in the range 10% by weight ethanol to 90% by weight ethanol were used as comparative solutions.

Furthermore, dilutions of the aforementioned emulsions without photosensitizer, as well as photosensitizer-containing emulsions were used, in which the relevant microemulsion was diluted in 5 steps to a water content of 99% by weight.

The contact angle was determined with the aid of the DataPhysics OCA 35 contact angle measuring instrument from DataPhysics Instruments GmbH (Filderstadt, DE), following the manufacturer's instructions.

For the measurement, 2.5 μL of each test solution was applied at room temperature with full climate control (temperature: 25° C., pressure: 1013 mbar, relative humidity: 50%) to a glass slide as the test surface, using an automatic Hamilton syringe in the form of a droplet and photographed at one second intervals.

Next, for each image, both the left and also the right contact angle between the droplet and the test surface was determined using SCA 20 software from DataPhysics Instruments GmbH, along with the mean of the measured contact angle. Each measurement was carried out 4 times.

FIG. 1 shows the mean of the measured contact angle for aqueous ethanol solutions with various ethanol concentrations in the range from 10% by weight of ethanol to 80% by weight of ethanol.

By way of example, FIG. 1 also shows the means of the measured contact angle for the photosensitizer-free microemulsions E1 to E4.

The means of the measured contact angle for microemulsions E1 to E4, which each contained 100 μm of one of the photosensitizers used, deviated only insignificantly from the measured contact angles for the photosensitizer-free microemulsions E1 to E4.

The various microemulsions with SDS and TWEEN® 20 exhibited a significantly reduced contact angle compared with pure water. More than 40% by weight of ethanol had to be used in order to obtain a comparable wetting of the glass surface employed.

The effect of the dilution of a microemulsion with water is shown by way of example in FIG. 2 on the photosensitizer-free microemulsion used (DMS; TWEEN® 20/1,2-pentanediol (1:3); water).

As can be seen in FIG. 2, microemulsion E3 can be diluted with an approximately 8-fold quantity of water without the contact angle of the dilution obtained increasing significantly in the test described above. Even a 16-fold dilution still exhibited sufficient wetting of the glass plate used in the test.

Similar results were obtained for microemulsions E1, E2 and E4 as well as for microemulsions E1 to E4, which respectively contained 5 μM of one of the photosensitizers TMPyP, SA-PN-01a, SA-PN-02a, SA-PN-24d, FL-AS-H-1a or FL-AS-H-2 employed.

C) UV/VIS Measurements

The absorption of the photosensitizers TMPyP, SA-PN-01a and FL-AS-H-1a used in the respective microemulsions E1 to E4 were determined by recording an absorption spectrum for a wavelength range of 250 nm to 600 nm.

In this regard, the photosensitizers SA-PN-01a and FL-AS-H-1a were dissolved in a concentration of 20 μM in water and in the respective microemulsions E1 to E4.

Because of the higher absorption of TMPyP in solution, the photosensitizer TMPyP was respectively used in a concentration von 5 μM.

Absorption spectra were measured using a Varian Cary BIO UV/VIS/IR spectrometer (Agilent Technologies Inc., Santa Clara, Calif., USA), wherein a 10 mm Hellma quartz cell (SUPRASIL, Type 101-QS, Hellma GmbH & Co. KG, Mühlheim, DE) was used.

The respective absorption spectra of TMPyP, SA-PN-01a and FL-AS-H-1a in the microemulsions E1 to E4 were almost identical, within the margin of error, to the corresponding absorption spectra of TMPyP, SA-PN-01a and FL-AS-H-1a in water.

There was no difference between the intensity of the signal, nor were there any modifications to the spectrum.

D) Determination of Singlet Oxygen Formed Following Irradiation

The formation of singlet oxygen following irradiation of a photosensitizer-containing microemulsion was determined using time-resolved singlet oxygen luminescence measurements.

For the relevant measurements, 5 μM of the respective photosensitizers used were dissolved in water or in the emulsions E1 to E4.

The time-resolved singlet oxygen luminescence measurements were carried out in accordance with the methods described in S. Y. Egorov et al., 1999.

A tuneable laser system was used to produce the singlet oxygen (model: NT242-SH/SFG, serial number: PGD048) from EKSPLA (Vilnius, Lettland). A portion of the monochromatic laser beam produced was directed onto a photodiode which acted as a trigger signal for the time-correlated single photon measurement.

The other part of the laser beam was directed onto a 1 cm thick quartz cell (SUPRASIL, Type 101-QS, Hellma GmbH & Co. KG, Mühlheim, DE), in which the solution to be tested had been disposed.

The formation of singlet oxygen was detected by direct detection of the time- and spectrally-resolved singlet oxygen luminescence.

Singlet oxygen luminescence was carried out by means of a nitrogen-cooled photomultiplier (model R5509-42, Hamamatsu Photonics, Hamamatsu, Japan) and a multiscaler (7886S, FAST Corn Tec GmbH, Oberhaching, Germany).

The singlet oxygen luminescence was detected at a wavelength in the range 1200 nm to 1400 nm using interference filters which were disposed in front of the photomultiplier.

The time-resolved singlet oxygen spectra are shown in FIGS. 3 to 5 by way of example for the respective photosensitizers TMPyP, SA-PN-01a and FL-AS-H-1a.

FIG. 3 shows the measured time-resolved singlet oxygen spectra for the photosensitizer TMPyP in a concentration of respectively 5 μM, in water (w), microemulsion E1 (E1) or microemulsion E2 (E2). FIG. 4 shows the measured time-resolved singlet oxygen spectra for the photosensitizer SA-PN-01a in a concentration of respectively 5 μM, in water (w), microemulsion E1 (E1) or microemulsion E2 (E2). FIG. 5 shows the measured time-resolved singlet oxygen spectra for the photosensitizer FL-AS-H-1a in a concentration of respectively 5 μM, in water (w), microemulsion E1 (E1) or microemulsion E2 (E2).

A summary of the singlet oxygen detection is shown in Table 1.

Each of the photosensitizers used, TMPyP, SA-PN-01a and FL-AS-H-1a, produced singlet oxygen, following irradiation with electromagnetic radiation. The quantum yield was determined in accordance with the method described in Baier J. et al. (“Singlet Oxygen Generation by UVA Light Exposure of Endogenous Photosensitizers”, Biophys. J. 91(4), 2006, pages 1452 to 1459; doi. 10.1529/biophysj.106.082388).

The singlet oxygen formed in the respective microemulsion exhibited a significantly longer half-life compared with water. The microemulsion almost doubled the half-life of the singlet oxygen compared with the half-life for the singlet oxygen formed in water, which was approximately 3.5 μs.

The relative yield of singlet oxygen for each photosensitizer with respect to the quantity of singlet oxygen formed in water was calculated from the ratio of the integrals.

The quantum yield of singlet oxygen in the microemulsions is at least twice as high as in water.

The formation of singlet oxygen in the microemulsions used was 5-times higher with FL-AS-H-1a and in fact 7 times higher with SA-PN-01a than in water.

TABLE 1
Results for singlet oxygen measurements for the photosensitizers
TMPyP, SA-PN-01a and TMPyP (each 5 μM) in water, microemulsion
E1 (DMS; SDS/1-pentanol (1:2); water) and microemulsion
E2 (DMS; SDS/1,2-pentanediol (1:2); water).

| | | | Decay | | Relative yield |
|---|---|---|---|---|---|
| Photo- | | Formation | period | | with respect |
| sensitizer | Solvent | time (μs) | (μs) | Integral | to H₂O |
| TMPyP | H₂O | 1.9 | 3.8 | 985 | — |
| TMPyP | E1 | 1.7 | 8.9 | 2874 | 2.92 |
| TMPyP | E2 | 2.3 | 7.2 | 1955 | 1.98 |
| SA-PN-01a | H₂O | 2.5 | 3.2 | 571 | — |
| SA-PN-01a | E1 | 1.0 | 9.2 | 4413 | 7.73 |
| SA-PN-01a | E2 | 1.6 | 7.4 | 3933 | 6.89 |
| FL-AS-H-1a | H₂O | 3.6 | 3.6 | 366 | — |
| FL-AS-H-1a | E1 | 3.0 | 8.6 | 1752 | 4.79 |
| FL-AS-H-1a | E2 | 4.0 | 6.7 | 1695 | 4.63 |

In summary, it can be seen that the use of a microemulsion has a positive influence on the photophysics of the photosensitizer used.

Significantly larger quantities of singlet oxygen were formed in one of the microemulsions used and the light absorption of the respective photosensitizers used in the microemulsion remained essentially unchanged.

E) Phototoxicity Measurements

In order to investigate the phototoxicity of the microemulsions in accordance with the invention, a MTT test was used. Assaying cell vitality using a MTT test is based on the reduction of the yellow, water-soluble dye 3-(4,5-dimethylthiazol-2-yl)-2,5-diphenyltetrazolium bromide (MIT, Sigma-Aldrich Chemie GmbH, Munich, DE) into a blue-violet 2,3,5-triphenyltetrazolium chloride (formazan) which is insoluble in water. MTT is a dye which can pass through membranes, which is metabolized by mitochondrial dehydrogenases in living cells, which in the end leads to the formation of formazan crystals.

Formazan crystals can no longer pass through the membranes and accumulate in proliferating undamaged cells. After cell lysis and dissolving the crystals, the dye is then quantified by colorimetric measurement at 550 nm in a multi-well spectrophotometer (ELISA reader). The quantity of formazan formed is determined as the optical density (OD). The measured quantity of formazan is directly proportional to the number of proliferating cells, so that this test is suitable for the measurement of the phototoxicity of the microemulsions used. The measured OD can be assigned a cell count by means of a previously determined calibration curve.

The concentration of the respective photosensitizers TMPyP, SA-PN-01a, SA-PN-02a, SA-PN-24d, FL-AS-H-1a or FL-AS-H-2 in the microemulsions E1 to E4 was 0 μM, 10 μM, 25 μM, 50 μM, 100 μM, 250 μM and 500 μM.

Furthermore, the respective microemulsions E1 to E4 without photosensitizer were used as a control.

The phototoxicity measurements were carried out on Escherichia coli (E. coli; ATCC Number: 25922) and Staphylococcus aureus (S. aureus; ATCC Number: 25923), as described by Mosmann (1983). (Mosmann T.: Rapid colorimetric assay for cellular growth and survival: application to proliferation and cytotoxicity assays: J. Immunol. methods. 1983 (65); pages 55-63).

25 μL of a suspension of the bacteria used were grown overnight in Müller-Hinton liquid medium (Merck KGaA, Darmstadt, Germany) with an optical density of 0.6 at 600 nm were incubated with 25 μL of the test solution at room temperature for 10 seconds in darkness in a 96-well microtitre plate (Cellstar, Greiner Bio-One, Frickenhausen, Germany).

Next, the microtitre plate was irradiated for 40 s. For irradiation, the light source Blue V from Waldmann (Villingen-Schwenningen, Germany) was used, which emits light at 380 to 480 nm (emission maximum at approximately 420 nm). The applied power was 20 mW/cm².

For each experiment, three controls were carried out at the same time in order to exclude side effects of the irradiation/photosensitizer (PS) on survival of the bacteria: (i) no PS, only light (=light control), (ii) no light, only PS (=dark control), and (iii) neither light nor PS (=reference control).

After irradiation had been completed, 75 μL of a 25% by weight SDS solution was added to each well of the microtitre plate and the bacterial cells were lysed overnight at 37° C. in an incubator.

Finally, the optical density (OD) was determined with the aid of a microtitre plate photometer (model EAR 400 AT, SLT Laborinstruments Austria, Salzburg, AT).

After lysis of the cells and dissolution of the crystals, the dye could then be quantified in a multi-well spectrophotometer (ELISA-Reader) by colorimetric measurement at 550 nm.

The determination of the colony forming units was carried out in accordance with the method published by Miles and Misra (Miles, AA; Misra, SS, Irwin, JO (1938 November). “The estimation of the bactericidal power of the blood” The Journal of hygiene 38 (6): 732-49). In this regard, serial dilutions from 10⁻² to 10⁻⁹ of the corresponding bacterial suspension were produced. In each case, 3×20 μL of the corresponding bacterial dilutions were then dropped onto Müller-Hinton plates and incubated at 37° C. for 24 h. Next, the number of surviving colony forming units (CFU) was determined. All of the experiments were carried out three times.

E. coli and S. aureus were destroyed by the singlet oxygen formed by the irradiation in a concentration range of 10 μM to 100 μM of the photosensitizer TMPyP both in water and in the microemulsions E1 to E4 used.

A shielding effect occurred at a concentration of more than 100 μM of the photosensitizer TMPyP in water. TMPyP can absorb 25 to 30 times more light. Thus, the formation of singlet oxygen at high concentrations is more than 100 μM less and corresponding concentrated aqueous solutions could reduce the quantity of E. Coli and S. Aureus only by 2 log units.

In contrast, when using TMPyP in one of the microemulsions E1 to E4, significantly less shielding occurred. Thus, the quantity of singlet oxygen formed at high concentrations of TMPyP (more than 100 μM to 500 μM) is higher compared with aqueous solutions.

Corresponding concentrated microemulsions with TMPyP in a concentration of more than 100 μM to 500 μM could reduce the quantity of E. coli and S. aureus by only 5 log₁₀ units.

The photosensitizers SA-PN-01a, SA-PN-02a and SA-PN-24d were more effective against E. coli and S. aureus when used in a microemulsion than when used in water.

S. aureus was completely destroyed in water (reduction in quantity following irradiation of more than 6 log₁₀ units) when SA-PN-01a, SA-PN-02a and SA-PN-24d were used in a concentration in the range 50 to 500 μM.

When using SA-PN-01a in one of the microemulsions E1 to E4, even from a concentration of 25 μM of SA-PN-01a, a reduction in the quantity of E. coli and S. aureus following irradiation of more than 6 log₁₀ units was obtained.

Furthermore, a concentration of 10 μM SA-PN-01a in one of the microemulsions E1 to E4 was sufficient to obtain a reduction in the quantity of E. coli and S. aureus of 3 log 10 units following irradiation.

FIGS. 6a and 6b show the action of SA-PN-01a in water or SA-PN-01a in microemulsion E2 (E2) on Staphylococcus aureus, by way of example.

FIG. 6a shows the action of an aqueous solution of the photosensitizer SA-PN-01a in the given concentrations on Staphylococcus aureus following irradiation (hatched bars) with the light source Blue V (irradiation period: 40 s). The applied power was respectively 20 mW/cm.

As a control, two non-irradiated samples (black bars) were also included, in which Staphylococcus aureus was treated respectively with pure water without SA-PN-01a (concentration: 0 μM) or SA-PN-01a in water in a concentration of 500 μM.

FIG. 6b shows, by way of example, the action of the photosensitizer SA-PN-01a in microemulsion E2 in the concentrations given on Staphylococcus aureus following irradiation (hatched bars) with the light source Blue V (irradiation period: 40 s). The applied power was respectively 20 mW/cm.

As a control, two non-irradiated samples (black bars) were also included, in which Staphylococcus aureus was treated respectively with microemulsion E2 without SA-PN-01a (concentration: 0 μM) or SA-PN-01a in microemulsion E2 in a concentration of 500 μM.

The measured colony forming units of surviving bacteria are shown in each case using the test in accordance with the method published by Miles and Misra, shown in colony forming units per millilitre (CFU/mL).

For the photosensitizer FL-AS-H-1a, at a concentration of 10 μM FL-AS-H-1a in one of the microemulsions E1 to E4, a reduction in the quantity of E. coli and S. aureus of approximately 2 log₁₀ units was measured.

### Example 2

A) Production of Various Oil-Containing Microemulsions

In addition, the oil containing microemulsions E5 and E6 were produced.

The % by weight of the components of the microemulsions E5 to E6 given below are respectively with respect to the total weight of the corresponding microemulsion without photosensitizer.

Microemulsion E5:

66% by weight dodecane

29% by weight Lutensol AO7

5% by weight water

Microemulsion E6:

66% by weight paraffin oil

4% by weight water

10% by weight Lutensol AO 7

20% by weight Kosteran SQ/O VH

The surfactant Lutensol AO7 is commercially available from BASF SE (Ludwigshafen, DE).

Lutensol AO7 is an ethoxylated mixture of fatty acids containing 13 to 15 carbon atoms with an average of 7 ethyl oxide units (PEG 7).

The surfactant Kosteran SQ/O VH is commercially available from Dr. W. Kolb AG (Hedingen, CH). Kosteran SQ/O VH is a sorbitan-oleic acid ester with an average of 1.5 oleic acid molecules per molecule (sorbitan sesquioleate).

B) UV/VIS Measurements

The absorption of the FL-AS-H-1a photosensitizer used in the microemulsions E5 and E6 was determined by recording an absorption spectrum for a wavelength range of 250 nm to 600 nm, as described in Example 1. To this end, the FL-AS-H-2 photosensitizer was dissolved in a concentration of 10 μM in the microemulsions E5 and E6, as well as in water.

The absorption spectrum of FL-AS-H-2 in microemulsion E6 did not exhibit any displacement of the spectrum compared with the spectrum measured in water. Only the intensity of the absorption signal was higher than in water or in microemulsion E5.

Furthermore, an absorption spectrum of FL-AS-H-2 in microemulsions E5 and E6 as well as in water was measured following irradiation with varying doses of light.

For the irradiation, the light source Blue V from Waldmann, which emits light at 380 to 480 nm (emission maximum at approximately 420 nm) was used. The applied light dose was from 5.5 J to 990 J.

It was shown that the FL-AS-H-2 photosensitizer was degraded both in water as well as in the microemulsions E5 and E6. The degradation in water occurred significantly faster than in the respective microemulsion E5 or E6.

### Example 3

A) Production of Photosensitizer-Containing Gels

The following percentages by weight for the components of gels G1 to G3 are respectively with respect to the total weight of the original aqueous solution used.

Gel G1: (Comparative example—no surfactant)

| | Quantity |
|---|---|
| Component | [mL] |
| Carbopol SF-1 (4% by weight aqueous solution) | 6.25 |
| Sodium hydroxide (2% by weight aqueous solution) | 2 |
| Sodium chloride (10% by weight aqueous solution) | 4 |

Carbopol Aqua SF-1 polymer, an acrylate copolymer, obtained from Lubrizol Corporation (Wickliffe, Ohio, USA), was used as the gelling agent.

Gel G2:

| | Quantity |
|---|---|
| Component | [mL] |
| Carbopol SF-1 (4% by weight aqueous solution) | 6.25 |
| Sodium hydroxide (2% by weight aqueous solution) | 2 |
| Sodium chloride (20% by weight aqueous solution) | 2 |
| Brij 35 (6% by weight aqueous solution) | 2 |

Brij 35, a polyoxyethylene (23) lauryl ether, obtained from Merck KGaA (Darmstadt, DE), was used as the surfactant.

Gel G3:

| | Quantity |
|---|---|
| Component | [mL] |
| Carbopol SF-1 (4% by weight aqueous solution) | 6.25 |
| Sodium hydroxide (2% by weight aqueous solution) | 2 |
| Sodium chloride (20% by weight aqueous solution) | 2 |
| PLANTACARE 818 UP (6% by weight aqueous solution) | 2 |

PLANTACARE 818 UP, a C8 to C16 fatty alcohol glucoside of D-glucopyranose, obtained from BASF SE (Ludwigshafen, DE), was used as the surfactant.

According to the manufacturer, the distribution of the lengths of the fatty alcohol portion is as follows:

| | |
|---|---|
| C6 | max. 0.5% |
| C8 | 24-30% |
| C10 | 15-22% |
| C12 | 37-42% |
| C14 | 12-18% |
| C16 | max. 4% |

Firstly, the aforementioned quantity of a 2% by weight aqueous NaOH solution was added in portions to a corresponding quantity of a 4% by weight aqueous solution of Carbopol Aqua SF-1 in a graduated flask, with stirring. After a clear gel had been formed, the aforementioned quantity of a sodium chloride solution was added in order to adjust the viscosity.

Next, the respective aforementioned quantity of a 6% by weight aqueous solution of one of the aforementioned surfactants was added dropwise, with stirring.

The photosensitizer TMPyP used was added to the relevant gel in a final concentration of 100 μM.

The gels G1, G2 and G3, respectively with and without photosensitizer TMPyP, were transparent and exhibited pseudo-elastic behaviour.

Furthermore, the consistency of the gels G2 and G3 did not change after storage for 24 hours at 50° C. as well as at 0° C.

B) Contact Angle Test

The wetting of surfaces by the gels which were produced was determined with the aid of the contact angle test.

For the contact angle test, the gels mentioned above were used, without photosensitizer as well as photosensitizer-containing gels.

The contact angle test was carried out as described in Example 1, wherein a polyethylene test plate was used as the test surface.

By way of example, FIG. 7 shows the measured contact angle for the photosensitizer-free gels G2 and G3, in which the relevant quantity of the given surfactant was added. The measured contact angles for the respective photosensitizer-containing gels G2 and G3 were identical.

The measurements show that, for a proportion of 0.5% by weight with respect to the total weight of the gel, a minimum contact angle and thus a maximum wetting was obtained.

In order to detach any aggregates of bacteria present, the proportion of the surfactants was then raised to 1.0% by weight with respect to the total weight of the gel.

C) UV/VIS Measurements

The absorption of the TMPyP photosensitizer used in the respective gels G1 to G3 as well as in water was determined by recording an absorption spectrum for a wavelength range of 250 nm to 600 nm, as described in Example 1.

In this regard, the photosensitizer TMPyP was dissolved in a concentration of 10 μM in the gels G1 to G3 as well as in water.

The absorption spectrum of TMPyP in gels G1 to G3 did not exhibit any displacement of the spectrum compared with the spectrum measured in water.

D) Determination of Singlet Oxygen Formed Following Irradiation

The formation of singlet oxygen following irradiation of a photosensitizer-containing microemulsion was determined using time-resolved singlet oxygen luminescence measurements, as described in Example 1.

In gels G1, G2 and G3, in the presence of TMPyP (final concentration 10 μM), the formation of singlet oxygen could be detected following irradiation.

By way of example, FIG. 8 shows the time-resolved singlet oxygen spectrum for the photosensitizer TMPyP in gel G3. The measured rise time for the signal (t_{R}) was 2.7 μs.

The measured decay time for the signal (t_{D}) was 7.4 μs.

By way of example, FIG. 9 shows the wavelength-resolved singlet oxygen spectrum for the photosensitizer TMPyP in gel G3.

The distinct peak in the wavelength-resolved spectrum at 1270 nm definitively shows that singlet oxygen is formed by TMPyP in the gel following irradiation.

The measured decay time for the singlet oxygen signal in the gel (7.4 μs), compared with the measured decay time for the singlet oxygen signal in water (˜3.5 μs) was significantly longer, so that the singlet oxygen formed in one of the tested gels G1 to G3 was active for longer.

### LITERATURE

Butenandt J., Epple R., Wallenborn E.-U., Eker A. P. M., Gramlich V. and Carell T.: A comparative repair study of thymine- and uracil-photodimers with model compounds and a photolyase repair enzyme, Chem. Eur. J. 2000, Vol. 6, No. 1, pages 62-72,

Svoboda J., Schmaderer H. and Konig B.: Thiourea-enhanced flavin photooxidation of benzyl alcohol; Chem. Eur. J. 2008, 14, pages 1854-1865

Egorov S. Y., Krasnovsky A. A., Bashtanov M. Y., Mironov E. A., Ludnikova T. A. and Kritsky M. S.; Photosensitization of singlet oxygen formation by pterins and flavins. Time-resolved studies of oxygen phosphorescence under laser excitation. Biochemistry (Mosc) 1999, 64 (10), pages 1117-1121.

1. A dispersion, comprising:
(a) at least one photosensitizer,
(b) at least one liquid polar phase, and
(c) at least one surfactant, and
wherein the dispersion comprises a microemulsion, a gel or a mixture thereof, at a temperature in the range 2° C. to 50° C. and a pressure in the range 800 to 1200 mbar.

2. The dispersion as claimed in claim 1, wherein the photosensitizer is selected from the group which consists of phenalenones, curcumins, flavins, porphyrins, porphycenes, xanthene dyes, coumarins, phthalocyanines, phenothiazine compounds, anthracene dyes, pyrenes, fullerenes, perylenes and mixtures thereof.

3. The dispersion as claimed in claim 1, wherein the at least one liquid polar phase comprises at least one polar solvent.

4. The dispersion as claimed in claim 1, wherein the at least one surfactant is selected from the group which consists of non-ionic surfactants, anionic surfactants, cationic surfactants, amphoteric surfactants and mixtures thereof.

5. The dispersion as claimed in claim 4, wherein the cationic surfactants are selected from the group which consists of quaternary alkylammonium salts, esterquats, acylated polyamines, benzylammonium salts and mixtures thereof.

6. The dispersion as claimed in claim 4, wherein the non-ionic surfactants are selected from the group which consists of polyalkyleneglycol ethers, alkylglucosides, alkylpolyglycosides, alkylglyco side esters and mixtures thereof.

7. The dispersion as claimed in claim 4, wherein the anionic surfactants are selected from the group which consists of alkylcarboxylates, alkylsulphonates, alkylsulphates, alkylphoshates, alkylpolyglycolethersulphates, sulphonates of alkylcarboxylic acid esters, N-alkyl-sarcosinates and mixtures thereof.

8. The dispersion as claimed in claim 1, wherein the dispersion further comprises at least one liquid non-polar phase, wherein the at least one liquid non-polar phase comprises at least one non-polar solvent.

9. The dispersion as claimed in claim 1, wherein the dispersion further contains at least one alkanol containing 2 to 12 carbon atoms.

10. The dispersion as claimed in claim 1, wherein the dispersion at least comprises a microemulsion at a pressure in the range 800 to 1200 mbar and a temperature in the range 2° C. to 50° C.

11. The dispersion as claimed in claim 10, wherein the microemulsion is an O/W-microemulsion, a water-in-oil (W/O) microemulsion or a bicontinuous microemulsion.

12. The dispersion as claimed in claim 1, wherein the dispersion further contains at least one pH-regulating substance.

13. The dispersion as claimed in claim 1, wherein the dispersion further comprises at least one gelling agent which is selected from the group which consists of carboxyvinyl polymers, polyacrylamides, polyvinyl alcohols, acylated polyethylene amines, alginates, cellulose ethers and mixtures thereof.

14. The dispersion as claimed in claim 1, wherein the dispersion at least comprises a gel at a pressure in the range 800 to 1200 mbar and a temperature in the range 2° C. to 50° C.

15. A method for the photodynamic inactivation of microorganisms selected from the group which consists of viruses, archaeae, bacteria, bacterial spores, fungi, fungal spores, protozoa, algae and blood-borne parasites, wherein the method comprises contacting said microorganisms with the dispersion as claimed in claim 1.

16. The method as claimed in claim 15, wherein the method is used for the surface cleaning and/or surface coating of an article.

17. The method as claimed in claim 15, wherein the method is used for the surface cleaning and/or surface coating of at least one which is selected from the group which consists of medical products, food packaging, foodstuffs, beverage packaging, beverage containers, textiles, building materials, electronic devices, household appliances, furniture, windows, floors, walls pal and hygiene articles,

18. The method as claimed in claim 15, wherein the method is used for the decontamination of liquids.

19. A method for the photodynamic inactivation of microorganisms selected from the group consisting of viruses, archaeae, bacteria, bacterial spores, fungi, fungal spores, protozoa, algae and bloodborne parasites, wherein the method comprises the following steps:
(A) bringing the microorganisms into contact with a photosensitizer-containing dispersion as claimed in claim 1, and
(B) irradiating the microorganisms and the at least one photosensitizer with electromagnetic radiation of a suitable wavelength and energy density for inactivating the microorganisms.

20. A method of photodynamic therapy for the inactivation of microorganisms which are selected from the group which consists of viruses, archaeae, bacteria, bacterial spores, fungi, fungal spores, protozoa, algae and blood-borne parasites, wherein the method comprises contacting said microorganisms with the dispersion as claimed in claim 1.

21. The dispersion as claimed in claim 1, adapted for use during photodynamic therapy for the inactivation of microorganisms which are selected from the group which consists of viruses, archaeae, bacteria, bacterial spores, fungi, fungal spores, protozoa, algae and blood-borne parasites, in the treatment and/or prophylaxis of a disease of dental tissue and/or of the periodontium.
